(19) United States
(12) Patent Application Publication
(10) Pub. No.: US 2007/0060812 A1
(43) Pub. Date: Mar. 15, 2007

(54) Sensing of pancreatic electrical activity

(75) Inventors: Tamar Harel, Haifa (IL); Tamir Levi, Ein Haemek (IL); Yuval Mika, Shmurat Zichron (IL); Offer Glassberg, Haifa (IL); Nissim Darvish, Hof Hacarmel (IL); Radwan Khawaled, Shfar'am (IL); Shimon Marom, Haifa (IL)

Correspondence Address: WOLF, BLOCK, SHORR AND SOLIS-COHEN LLP, 250 PARK AVENUE, 10TH FLOOR, NEW YORK, NY, 10177, US

(73) Assignee: METACURE N.V., Willemstad , Curacao (NL)

(21) Appl. No.: 10/497,126

(22) PCT Filed: Oct. 24, 2002
(86) PCT No.: PCT/IL02/00856

§ 371 Date: May 17, 2005

Related U.S. Application Data
(60) Provisional application No. 60/334,017, filed on Nov. 29, 2001.

(51) Int. Cl. A61B 5/05

(52) U.S. Cl. 600/347

(57) ABSTRACT

Apparatus (18) is provided for sensing electrical activity of a pancreas (20) of a patient. The apparatus (18) includes a set of one or more electrodes (100), adapted to be coupled to the pancreas (20), and to generate activity signals indicative of electrical activity of pancreatic cells which are in a plurality of islets of the pancreas (20). The apparatus (18) also includes a control unit (90), adapted to receive the activity signals, and to generate an output signal responsive thereto.

### CROSS-REFERENCE TO RELATED APPLICATIONS

This application is a continuation-in-part of PCT Patent Application No. PCT/IL01/00501, filed May 30, 2001, entitled, “Electropancreatography,” which claims priority from U.S. Provisional Patent Application No. 60/208,157, filed May 31, 2000, entitled, “Electrical activity sensor for the whole pancreas.” The '501 and '157 applications are assigned to the assignee of the present patent application and incorporated herein by reference.

This application claims priority from U.S. Provisional Patent Application No. 60/334,017, filed Nov. 29, 2001, entitled, “In situ sensing of pancreatic electrical activity,” which is assigned to the assignee of the present patent application and incorporated herein by reference.

### FIELD OF THE INVENTION

The present invention relates generally to electrical sensing, and specifically to invasive devices and methods for sensing electrical activity of the pancreas.

### BACKGROUND OF THE INVENTION

The human pancreas performs two functions: producing pancreatic endocrine hormones, which affect the behavior of cells throughout the body, and producing pancreatic digestive enzymes, which assist in the digestion of food. Among other endocrine hormones produced by the pancreas, insulin is the most well-known, because of the large number of diabetic patients who regularly monitor their glucose levels to determine whether to self-administer a dose of insulin. The general function of insulin is to regulate blood glucose levels, by causing peripheral cells of the body to absorb glucose as a person's blood sugar rises. Some types of diabetes, for example, arise as a consequence of inadequate insulin release by the pancreas. Normal, physiological insulin generation and uptake, however, allow peripheral cells to properly manage the body's energy needs.

It is well known in the art to measure the electrical activity of individual pancreatic beta cells, for example, by micropipetting. It is also known to measure the collective activity of the cluster of cells in a pancreatic islet of Langerhans.

An article by Jaremko and Rorstad, entitled, “Advances toward the implantable artificial pancreas for treatment of diabetes,” Diabetes Care, 21(3), March 1998, which is incorporated herein by reference, describes enzymatic glucose sensors and optical glucose sensors for use in an artificial pancreas. They note that “ . . . implantable enzymatic sensors are not yet clinically applicable because of problems with biocompatibility. Clinical research is necessary on the effect of chronic subcutaneous implantation and local inflammation on glucose sensor performance.” Moreover, with respect to optical sensors, they write: “It appears that despite recent press releases, we are still some way from having a widely applicable long-term optical blood glucose sensor. This technology avoids the biocompatibility problems of enzymatic sensors but improvements in precision and reductions in cost are needed. Basic research is required as to the effects of environmental and metabolic variations on absorption spectra before a reliable and clinically practical optical sensor will become available.” They similarly describe subcutaneous microdialysis probes and a transcutaneous glucose extraction device as not yet being suitable for regular clinical use. They conclude, “the quest for a reliable, long-term, wearable, or implantable blood glucose sensor has been frustrating so far and few clinical studies have been carried out.”

PCT Publication WO 01/91854 to Harel et al., which is assigned to the assignee of the present patent application and is incorporated herein by reference, describes apparatus for sensing electrical activity of a pancreas, including one or more electrodes, adapted to be coupled to the pancreas, and a control unit, adapted to receive electrical signals from the electrodes indicative of electrical activity of pancreatic cells which are in a plurality of islets of the pancreas, and to generate an output responsive thereto.

U.S. Pat. Nos. 6,093,167 and 6,261,280 to Houben et al., which are incorporated herein by reference, describe implantable apparatus for monitoring pancreatic beta cell electrical activity in a patient in order to obtain a measure of the patient's insulin demand and blood glucose level. A stimulus generator delivers stimulus pulses, which are intended to synchronize pancreatic beta cell depolarization and to thereby produce an electrical response in the pancreas. This response is analyzed so as to determine an indication of insulin demand, whereupon insulin from an implanted pump is released, or the pancreas is stimulated so as to enhance insulin production.

U.S. Pat. No. 5,919,216 to Houben et al., which is incorporated herein by reference, describes a system for automatically responding to insulin demand without any need for external monitoring or injecting of insulin into a diabetic patient. The system as described senses glucose levels internally, and responds by stimulating either the pancreas or a transplant of pancreatic islets in order to enhance insulin production.

U.S. Pat. No. 5,741,211 to Renirie et al., which is incorporated herein by reference, describes apparatus which evaluates an electrocardiographic signal in order to determine an indication of blood insulin and/or glucose levels.

U.S. Pat. Nos. 5,101,814 and 5,190,041 to Palti, which are incorporated herein by reference, describe a system which utilizes implanted glucose-sensitive living cells to monitor blood glucose levels. The implanted cells produce a detectable electrical or optical signal in response to changes in glucose concentration in surrounding tissue. The signal is then detected and interpreted to give a reading indicative of blood glucose levels. U.S. Pat. No. 5,368,028 to Palti, which is incorporated herein by reference, describes a system which utilizes implanted chemo-sensitive living cells to monitor tissue or blood concentration levels of chemicals such as glucose.

The following articles, which are incorporated herein by reference, may be of interest. In particular, methods and apparatus described in one or more of these articles may be adapted for use with some preferred embodiments of the present invention.
1) Lamb F. S. et al., “Cyclosporine augments reactivity of isolated blood vessels,” Life Sciences, 40, pp. 2571-2578, 1987.
2) Johansson B. et al., “Static and dynamic components in the vascular myogenic response to passive changes in length as revealed by electrical and mechanical recordings from the rat portal vein,” Circulation Research, 36, pp. 76-83, 1975.
3) Zelcer E. et al., “Spontaneous electrical activity in pressurized small mesenteric arteries,” Blood Vessels, 19, pp. 301-310, 1982.
4) Schobel H. P. et al., “Preeclampsia—a state of sympathetic overactivity,” New England Journal of Medicine, 335, pp. 1480-1485, 1996.
5) Gomis A. et al., “Oscillatory patterns of electrical activity in mouse pancreatic islets of Langerhans recorded in vivo,” Pflugers Archiv European Journal of Physiology, Abstract Volume 432(3), pp. 510-515, 1996.
6) Soria B. et al., “Cytosolic calcium oscillations and insulin release in pancreatic islets of Langerhans,” Diabetes Metab., 24(1), pp. 3740, February 1998.
7) Magnus G. et al., “Model of beta-cell mitochondrial calcium handling and electrical activity. II. Mitochondrial variables,” American Journal of Physiology, 274(4 Pt 1): C1174-1184, April 1998.
8) Gut R. et al., “High-precision EMG signal decomposition using communication techniques,” IEEE Transactions on Signal Processing, 48(9), pp. 2487-2494, September 2000.
9) Nadal A. et al., “Homologous and heterologous asynchronicity between identified alpha-, beta-, and delta-cells within intact islets of Langerhans in the mouse,” Journal of Physiology, 517(Pt. 1), pp. 85-93, May 1999.
10) Rosenspire A. J. et al., “Pulsed DC electric fields couple to natural NAD(P)H oscillations in HT-1080 fibrosarcoma cells,” Journal of Cell Science, 114(Pt. 8), pp. 1515-1520, April 2001.

### SUMMARY OF THE INVENTION

It is an object of some aspects of the present invention to provide improved methods and apparatus for sensing pancreatic electrical activity.

It is also an object of some aspects of the present invention to provide methods and apparatus for sensing electrical activity of a substantial portion of the pancreas.

It is a further object of some aspects of the present invention to provide improved methods and apparatus for modifying pancreatic function.

It is yet a further object of some aspects of the present invention to provide improved methods and apparatus for treating physiological disorders resulting from improper functioning of the pancreas.

It is still a further object of some aspects of the present invention to provide improved methods and apparatus for monitoring glucose and/or insulin levels in the blood.

In preferred embodiments of the present invention, pancreatic apparatus comprises a control unit and one or more electrodes, adapted to be coupled to respective sites on, in, or near the pancreas of a human subject. Preferably, the electrodes convey to the control unit electrical signals which are generated within a substantial portion of the pancreas. Typically, but not necessarily, the control unit analyzes various aspects of the signals, and drives the electrodes to apply pancreatic control signals to the pancreas responsive to the analysis. The term “substantial portion of the pancreas,” as used in the context of the present patent application and in the claims, is to be understood as a portion of the pancreas larger than two or more islets. Typically, the portion includes ten or more islets.

By way of analogy, the behavior of the heart cannot be adequately summarized by assessing the electrical activity of any one bundle of cells; instead, an electrocardiogram is used. Some embodiments of the present invention, similarly, assess the electrical activity of a substantial portion of the pancreas, typically in order to determine whether a treatment is appropriate (e.g., stimulating the pancreas to secrete more insulin, or generating a signal to activate an implanted insulin pump). For this reason, the inventors call the process of sensing the electrical activity of a substantial portion of the pancreas, as described herein, electropancreatography (EPG). Experiments performed by the inventors have shown that electropancreatography is sensitive to clinically-significant phenomena, e.g., an increase in blood glucose- and/or insulin levels from normal to supraphysiological values.

In some preferred embodiments, the control unit drives some or all of the electrodes to apply signals to the pancreas responsive to detecting EPG signals which are indicative of a particular physiological condition, such as elevated blood glucose and/or insulin levels. Preferably, these signals are applied using methods and apparatus similar to those described in one or more of the following applications/publications: (a) U.S. Provisional Patent Application 60/123,532, filed Mar. 5, 1999, entitled “Modulation of insulin secretion,” (b) PCT Publication WO 00/53257 to Darwish et al., and the corresponding U.S. patent application Ser. No. 09/914,889, filed Sep. 4, 2001, or (c) PCT Publication WO 01/66183 to Darvish et al., and the corresponding U.S. patent application Ser. No. 10/237,263, filed Sep. 5, 2002, all of which are assigned to the assignee of the present patent application and are incorporated herein by reference. Typically, each electrode conveys a particular waveform to the pancreas, which may differ in certain aspects from the waveforms applied to other electrodes. The particular waveform to be applied to each electrode is preferably determined by the control unit, initially under the control of a physician during a calibration period of the unit. After the initial calibration period, the unit is generally able to automatically modify the waveforms as needed to maintain a desired level of performance of the apparatus.

In some preferred embodiments, one or more physiological sensors (e.g., for measuring blood sugar, blood pH, pCO2, pO2, blood insulin levels, blood ketone levels, ketone levels in expired air, blood pressure, respiration rate, respiration depth, a metabolic indicator (e.g., NADH), or heart rate) send physiological-sensor signals to the control unit. The various sensor signals serve as feedback, to enable the control unit to iteratively adjust the signals applied to the pancreas. Alternatively or additionally, other sensors are coupled to the pancreas or elsewhere on the patient's body, and send signals to the control unit which are used in determining modifications to parameters of the applied signals.

As appropriate, methods and apparatus described in U.S. Provisional Patent Application 60/208,157, entitled, “Electrical Activity Sensor for the Whole Pancreas,” filed May 31, 2000, which is assigned to the assignee of the present patent application and is incorporated herein by reference, may be adapted for use with embodiments of the present invention. Alternatively or additionally, methods and apparatus described in the above-cited PCT Publication WO 01/91854 to Harel et al., may be adapted for use with embodiments of the present invention.

In some preferred embodiments of the present invention, one or more of the electrodes comprise wire electrodes fixed to a clip mount. For some applications, each wire electrode is looped through two holes in the clip, so that the curved portion of the wire electrode is exposed to the surface of the skin. Alternatively, the end of the wire electrode penetrates the pancreas.

In some preferred embodiments, one or more of the electrodes is fixed to a patch, which is coupled to tissue of the patient. For some applications, the electrodes comprise a monopolar wire electrode surrounded by an insulating ring. Preferably a patch comprises two such electrodes. Alternatively, the electrodes comprise concentric electrode assemblies, comprising an inner wire electrode and an outer ring electrode, with an inner insulating ring separating the inner wire electrode and the outer ring electrode. The assemblies preferably also comprise an outer insulating ring surrounding the outer ring electrode. Preferably, but not necessarily, the surface areas of the inner wire electrode and the outer ring electrode in contact with the tissue are within between about 2% and about 5% of each other, and, for some applications, are substantially equal.

In some preferred embodiments, the electrodes comprise sets of two button-electrodes attached to a preamplifier fixed to a patch. One end of a wire is connected to each electrode, and the other end of the wire comprises a needle, which is used to suture the electrode to the tissue. After suturing, the needle is preferably broken, and the remaining portion of the needle is inserted into the preamplifier. The patch is then coupled to the tissue at a distance from the suture site in the tissue selected so as to keep the wire moderately slack, thereby avoiding disturbing of the electrode during movement of the tissue.

In some preferred embodiments, the pancreatic apparatus comprises a signal-processing patch assembly, for implantation on the pancreas. The patch assembly preferably comprises one or more electrodes, and signal-processing components, such as a preamplifier, filters, amplifiers, a preprocessor, and a transmitter, some or all of which are preferably physically located on the patch assembly. Alternatively, the patch assembly does not comprise any electrodes, and electrodes are implanted in a vicinity of the patch and electrically coupled to the patch, which may be implanted on the pancreas or near the pancreas, such as on the duodenum.

There is therefore provided, in accordance with a preferred embodiment of the present invention, apparatus for sensing electrical activity of a pancreas of a patient, including:

a set of one or more electrodes, adapted to be coupled to the pancreas, and to generate activity signals indicative of electrical activity of pancreatic cells which are in a plurality of islets of the pancreas; and

a control unit, adapted to receive the activity signals, and to generate an output signal responsive thereto.

In an embodiment, a single electrode in the set of one or more electrodes is adapted to convey to the control unit an activity signal indicative of electrical activity of pancreatic cells which are in two or more of the islets.

There is also provided, in accordance with a preferred embodiment of the present invention, apparatus for analyzing electrical activity of a pancreas of a patient, including:

a set of one or more electrodes, each electrode adapted to be coupled to the pancreas and to generate an activity signal indicative of electrical activity of pancreatic cells which are in a plurality of islets of the pancreas; and

a control unit, adapted to:

receive the activity signals from the one or more electrodes,

analyze the received activity signals, and

generate an output signal responsive to the analysis.

In an embodiment, the set of electrodes is adapted to generate activity signals indicative of electrical activity of pancreatic cells which are in five or more of the islets. In an embodiment, the set of electrodes is adapted to generate activity signals indicative of electrical activity of pancreatic cells which are in ten or more of the islets.

In an embodiment, a first one of the one or more electrodes is adapted to generate a first activity signal, indicative of electrical activity of pancreatic cells which are in a first one of the islets, and a second one of the one or more electrodes is adapted to generate a second activity signal, indicative of electrical activity of pancreatic cells which are in a second one of the islets, which is different from the first one of the islets, and the control unit is adapted to receive the first and second activity signals.

In an embodiment, the control unit is adapted to analyze the activity signals so as to identify an aspect thereof indicative of activity of a type of cell selected from the list consisting of: pancreatic alpha cells, pancreatic beta cells, pancreatic delta cells, and polypeptide cells, and the control unit is adapted to generate the output signal responsive to identifying the aspect.

There is further provided, in accordance with a preferred embodiment of the present invention, apparatus for monitoring a blood glucose level of a patient, including:

a set of one or more electrodes, adapted to be coupled to a pancreas of the patient, and to generate respective activity signals indicative of spontaneous electrical activity of pancreatic cells; and

a control unit, adapted to receive the respective activity signals, to analyze the activity signals so as to determine a change in the glucose level, and to generate an output signal responsive to determining the change.

There is still further provided, in accordance with a preferred embodiment of the present invention, apparatus for monitoring a blood insulin level of a patient, including:

a set of one or more electrodes, adapted to be coupled to a pancreas of the patient, and to generate respective activity signals indicative of spontaneous electrical activity of pancreatic cells; and

a control unit, adapted to receive the respective activity signals, to analyze the activity signals so as to determine a change in the insulin level, and to generate an output signal responsive to determining the change.

In an embodiment, the control unit is adapted to analyze the activity signals so as to identify an aspect thereof indicative of activity of a type of cell selected from the list consisting of: pancreatic alpha cells, pancreatic beta cells, pancreatic delta cells, and polypeptide cells, and the control unit is adapted to generate the output signal responsive to identifying the aspect.

In an embodiment, the control unit is adapted to analyze the activity signals so as to identify a frequency aspect thereof, and to generate the output signal responsive to identifying the frequency aspect.

There is also provided, in accordance with a preferred embodiment of the present invention, apparatus for analyzing electrical activity of a pancreas of a patient, including:

a set of one or more electrodes, adapted to be coupled to the pancreas, and to generate activity signals; and

a control unit, adapted to receive the activity signals, adapted to analyze the activity signals so as to identify an aspect thereof which is indicative of activity of pancreatic alpha cells, and adapted to generate an output signal responsive to identifying the aspect.

There is additionally provided, in accordance with a preferred embodiment of the present invention, apparatus for analyzing electrical activity of a pancreas of a patient, including:

a set of one or more electrodes, adapted to be coupled to the pancreas, and to generate activity signals; and

a control unit, adapted to receive the activity signals, adapted to analyze the activity signals so as to identify an aspect thereof which is indicative of activity of pancreatic beta cells, and adapted to generate an output signal responsive to identifying the aspect.

In an embodiment, the control unit is adapted to analyze the activity signals so as to distinguish between the aspect thereof which is indicative of the activity of the beta cells and an aspect thereof which is indicative of activity of pancreatic alpha cells, and the control unit is adapted to generate the output signal responsive to distinguishing between the aspects.

There is yet additionally provided, in accordance with a preferred embodiment of the present invention, apparatus for analyzing electrical activity of a pancreas of a patient, including:

a set of one or more electrodes, adapted to be coupled to the pancreas, and to generate activity signals; and

a control unit, adapted to receive the activity signals, adapted to analyze the activity signals so as to identify an aspect thereof which is indicative of activity of pancreatic delta cells, and adapted to generate an output signal responsive to identifying the aspect.

There is still additionally provided, in accordance with a preferred embodiment of the present invention, apparatus for analyzing electrical activity of a pancreas of a patient, including:

a set of one or more electrodes, adapted to be coupled to the pancreas, and to generate activity signals; and

a control unit, adapted to receive the activity signals, adapted to analyze the activity signals so as to identify an aspect thereof which is indicative of activity of polypeptide cells, and adapted to generate an output signal responsive to identifying the aspect.

In an embodiment, the control unit is adapted to compare the aspect of the activity signals with a stored pattern that is indicative of activity of the cells, and to generate the output signal responsive thereto.

In an embodiment, the control unit is adapted to analyze the activity signals under an assumption that the activity of the cells is dependent on electrical activity of another type of pancreatic cell, and to generate the output signal responsive thereto.

In an embodiment, the control unit is adapted to analyze the activity signals under an assumption that the activity of the cells is substantially independent of electrical activity of another type of pancreatic cell, and to generate the output signal responsive thereto.

In an embodiment, the control unit is adapted to analyze the activity signals so as to identify a frequency aspect thereof, and to generate the output signal responsive to identifying the frequency aspect.

In an embodiment, the control unit is adapted to analyze the activity signals so as to differentiate between a first frequency aspect of the activity signals which is indicative of the activity of the cells, and a second frequency aspect of the activity signals, different from the first frequency aspect, which is indicative of activity of another type of pancreatic cell.

In an embodiment, the control unit is adapted to analyze the activity signals so as to identify over time a change in the frequency aspect that is characteristic of the cells.

In an embodiment, the control unit is adapted to analyze the activity signals so as to identify a magnitude aspect thereof, the control unit is adapted to analyze the frequency aspect and the magnitude aspect in combination, and the control unit is adapted to generate the output signal responsive to analyzing the aspects.

In an embodiment, the control unit is adapted to analyze the activity signals so as to identify a duration aspect thereof, the control unit is adapted to analyze the frequency aspect and the duration aspect in combination, and the control unit is adapted to generate the output signal responsive to analyzing the aspects.

In an embodiment, the set of electrodes is adapted to generate the activity signals responsive to spontaneous electrical activity of the pancreatic cells. In an embodiment, the control unit is adapted to apply a synchronizing signal to the pancreas.

In an embodiment, the control unit is adapted to analyze the activity signals so as to identify a magnitude of a fluctuation of the activity signals, and to generate the output signal responsive to the analysis.

In an embodiment, the control unit is adapted to analyze the activity signals by means of a technique selected from the list consisting of: single value decomposition and principal component analysis, and to generate the output signal responsive thereto.

In an embodiment, the control unit is adapted to analyze the activity signals so as to identify a duration aspect thereof, and to generate the output signal responsive to identifying the duration aspect.

In an embodiment, the control unit is adapted to analyze the activity signals so as to identify an aspect of morphology of a waveform thereof, and to generate the output signal responsive to identifying the aspect of the morphology.

In an embodiment, the control unit is adapted to analyze the activity signals so as to identify an aspect of a number of threshold-crossings thereof, and to generate the output signal responsive to identifying the aspect of the number of threshold-crossings.

In an embodiment, the control unit is adapted to analyze the activity signals using a moving window, and to generate the output signal responsive to the analysis.

In an embodiment, the control unit is adapted to analyze the activity signals so as to identify a measure of energy thereof, and to generate the output signal responsive to identifying the measure of energy.

In an embodiment, the control unit is adapted to analyze the activity signals so as to identify a correlation thereof with a stored pattern, and to generate the output signal responsive to identifying the correlation.

In an embodiment, the control unit is adapted to analyze the activity signals so as to determine an average pattern thereof, and so as to identify a correlation of the activity signals with the average pattern, and the control unit is adapted to generate the output signal responsive to identifying the correlation.

In an embodiment, the control unit is adapted to analyze the activity signals so as to identify a magnitude aspect thereof and a duration aspect thereof, the control unit is adapted to analyze the aspects in combination, and the control unit is adapted to generate the output signal responsive to analyzing the aspects.

In an embodiment, the control unit is adapted to analyze the activity signals so as to determine a measure of organization of the activity signals.

In an embodiment, a first electrode and a second electrode of the set of electrodes are adapted to be coupled to a first site and a second site of the pancreas, respectively, and the control unit is adapted to measure a delay between sensed electrical activity at the first and second sites, and to analyze the activity signals responsive to the measured delay.

In an embodiment, the control unit is adapted to detect mechanical artifacts by identifying a pattern of the activity signals, the pattern selected from the list consisting of: a spectral pattern and a time pattern.

In an embodiment, the control unit includes a memory, and the control unit is adapted to store the activity signals in the memory for subsequent off-line analysis.

In an embodiment, the control unit is adapted to receive the activity signals from at least one of the electrodes when the at least one of the electrodes is not in physical contact with any islet of the pancreas.

In an embodiment, the control unit is adapted to receive the activity signals from at least one of the electrodes when the at least one of the electrodes is not in physical contact with the pancreas.

In an embodiment, the control unit is adapted to generate the output signal so as to facilitate an evaluation of a state of the patient.

In an embodiment, the set of electrodes includes at least ten electrodes. In an embodiment, the set of electrodes includes at least 50 electrodes.

In an embodiment, the apparatus includes a clip mount, coupled to at least one of the electrodes, which is adapted for securing the at least one of the electrodes to the pancreas.

In an embodiment, at least one of the electrodes is adapted to be physically coupled to the pancreas by peeling back a portion of connective tissue surrounding the pancreas, so as to create a pocket, inserting the electrode into the pocket, and suturing the electrode to the connective tissue.

In an embodiment, the set of one or more electrodes includes an array of electrodes, the array including at least two electrodes adapted to be coupled to the pancreas at respective sites, and adapted to generate an impedance-indicating signal responsive to a level of electrical impedance between the two sites.

In an embodiment, the apparatus includes at least one supplemental sensor, adapted to be coupled to a site of a body of the patient, sense a parameter of the patient, and generate a supplemental signal responsive to the parameter, and the control unit is adapted to receive the supplemental signal. In an embodiment, the parameter is selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate, and the supplemental sensor is adapted to sense the parameter. In an embodiment, the metabolic indicator includes a measure of NADH, and the supplemental sensor is adapted to sense the measure of NADH. In an embodiment, the supplemental sensor includes an accelerometer, adapted to detect a motion of an organ of the patient. In an embodiment, the control unit is adapted to apply to the activity signals a noise reduction algorithm, an input of which includes the supplemental signal.

In an embodiment, the control unit is adapted to analyze the activity signals so as to identify a magnitude aspect thereof, and to generate the output signal responsive to identifying the magnitude aspect. In an embodiment, the control unit is adapted to analyze the activity signals so as to identify the magnitude aspect thereof at a frequency, and to generate the output signal responsive to identifying the magnitude aspect at the frequency.

In an embodiment, the control unit is adapted to apply a Fourier transform to the activity signals. In an embodiment, the control unit is adapted to analyze the Fourier-transformed activity signals so as to calculate a ratio of (a) a first frequency component at a first frequency of the activity signals to (b) a second frequency component at a second frequency of the activity signals, the first frequency different from the second frequency, and the control unit is adapted to generate the output signal responsive to the analysis. In an embodiment, the control unit is adapted to analyze the Fourier-transformed activity signals so as to identify a pattern thereof, and to generate the output signal responsive to identifying the pattern.

In an embodiment, the control unit is adapted to analyze the activity signals so as to identify an aspect of a frequency of spike generation thereof, and to generate the output signal responsive to identifying the aspect. In an embodiment, the control unit is adapted to analyze the activity signals so as to identify the aspect of the frequency of spike generation responsive to an occurrence of spikes within a certain range of durations of spikes, and to generate the output signal responsive to the aspect. In an embodiment, the control unit is adapted to analyze the activity signals so as to identify the aspect of the frequency of spike generation responsive to a ratio of spikes with a first amplitude to spikes with a second amplitude, the first amplitude different from the second amplitude, and to generate the output signal responsive to the aspect. In an embodiment, the control unit is adapted to analyze the activity signals so as to identify the aspect of the frequency of spike generation responsive to, for each spike, a product of a duration of the spike and an amplitude of the spike, and to generate the output signal responsive to the aspect. In an embodiment, the control unit is adapted to analyze the activity signals so as to identify a change in the aspect of the frequency of spike generation, and to generate the output signal responsive to identifying the change in the aspect of the frequency.

In an embodiment, the control unit is adapted to analyze the activity signals so as to determine a change in a rate of secretion of insulin by the pancreas. In an embodiment, the control unit is adapted to determine a change in a rate of spike generation, so as to determine the change in the rate of secretion of insulin by the pancreas.

In an embodiment, the control unit is adapted to analyze the activity signals with respect to calibration data indicative of aspects of pancreatic electrical activity recorded at respective times, in which respective measurements of a parameter of the patient generated respective values. In an embodiment, the parameter includes a blood glucose level of the patient, and the control unit is adapted to analyze the activity signals with respect to the calibration data. In an embodiment, the parameter includes a blood insulin level of the patient, and the control unit is adapted to analyze the activity signals with respect to the calibration data.

In an embodiment, the apparatus includes at least one reference electrode, adapted to be coupled to tissue in a vicinity of the pancreas, and to generate reference signals, and the control unit is adapted to receive the reference signals, and to generate the output signal responsive to the reference signals and the activity signals. In an embodiment, the reference electrode is adapted to be coupled to an organ of the patient in a vicinity of the pancreas, and to generate reference signals indicative of a motion of the organ. In an embodiment, the organ includes a stomach of the patient, and the reference electrode includes two reference electrodes, adapted to be coupled to the stomach at respective stomach sites, and adapted to generate an impedance-indicating signal responsive to a level of electrical impedance between the two stomach sites. In an embodiment, the organ includes a pancreas of the patient, and the reference electrode includes two reference electrodes, adapted to be coupled to the pancreas at respective pancreas sites, and adapted to generate an impedance-indicating signal responsive to a level of electrical impedance between the two pancreas sites. In an embodiment, the organ includes a duodenum of the patient, and the reference electrode includes two reference electrodes, adapted to be coupled to the duodenum at respective duodenum sites, and adapted to generate an impedance-indicating signal responsive to a level of electrical impedance between the two duodenum sites.

In an embodiment, the electrodes are adapted to be placed in physical contact with the pancreas. In an embodiment, at least one of the electrodes is adapted to be placed in physical contact with the head of the pancreas. In an embodiment, at least one of the electrodes is adapted to be placed in physical contact with the body of the pancreas. In an embodiment, at least one of the electrodes is adapted to be placed in physical contact with the tail of the pancreas. In an embodiment, at least one of the electrodes is adapted to be placed in physical contact with a vein or artery of the pancreas. In an embodiment, at least one of the electrodes is adapted to be placed in physical contact with a blood vessel in a vicinity of the pancreas.

In an embodiment, at least one of the electrodes has a characteristic diameter less than about 3 millimeters. In an embodiment, the at least one of the electrodes has a characteristic diameter less than about 300 microns. In an embodiment, the at least one of the electrodes has a characteristic diameter less than about 30 microns.

In an embodiment, the apparatus includes a treatment unit, adapted to receive the output signal and to apply a treatment to the patient responsive to the output signal.

In an embodiment, the control unit is adapted to generate the output signal responsive to an aspect of timing of the activity signals, and the treatment unit is adapted to apply the treatment responsive to the timing aspect. In an embodiment, the control unit is adapted to generate the output signal responsive to an aspect of the timing of the activity signals indicative of a phase in an oscillation of an insulin level.

In an embodiment, including at least one supplemental sensor, adapted to

be coupled to a site of a body of the patient,

sense a parameter of the patient, and

generate a supplemental signal responsive to the parameter,

and the control unit is adapted to receive the supplemental signal, and to generate the output signal responsive to the supplemental signal and the activity signals, and the treatment unit is adapted to apply the treatment responsive to the output signal. In an embodiment, the supplemental sensor includes an accelerometer, adapted to detect a motion of an organ of the patient. In an embodiment, the parameter is selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate, and the supplemental sensor is adapted to sense the parameter. In an embodiment, the metabolic indicator includes a measure of NADH, and the supplemental sensor is adapted to sense the measure of NADH.

In an embodiment, the control unit is adapted to configure the output signal to the treatment unit so as to be capable of modifying an amount of glucose in blood in the patient. In an embodiment, the control unit is adapted to configure the output signal to the treatment unit so as to be capable of increasing an amount of glucose in blood in the patient. In an embodiment, the control unit is adapted to configure the output signal so as to be capable of decreasing an amount of glucose in blood in the patient.

In an embodiment, the treatment unit includes a signal-application electrode, and the control unit is adapted to drive the signal-application electrode to apply current to the pancreas capable of treating a condition of the patient. In an embodiment, the signal-application electrode includes at least one electrode of the set of electrodes. In an embodiment, the control unit is adapted to drive the signal-application electrode to apply the current in a waveform selected from the list consisting of: a monophasic square wave pulse, a sinusoid wave, a series of biphasic square waves, and a waveform including an exponentially-varying characteristic. In an embodiment, the signal-application electrode includes a first and a second signal-application electrode, and the control unit is adapted to drive the first and second signal-application electrodes to apply the current in different waveforms. In an embodiment, the control unit is adapted to drive the signal-application electrode to apply the current so as to modulate insulin secretion by the pancreas.

In an embodiment, the control unit is adapted to select a parameter of the current, and to drive the signal-application electrode to apply the current, so as to modulate insulin secretion, the parameter selected from the list consisting of: a magnitude of the current, a duration of the current, and a frequency of the current. In an embodiment, the signal-application electrode includes a first and a second signal-application electrode, and the control unit is adapted to drive the first and the second signal-application electrodes to reverse a polarity of the current applied to the pancreas so as to stimulate the change in insulin secretion.

In an embodiment, the treatment unit includes a substance delivery unit, adapted to deliver a therapeutic substance to the patient, and the control unit is adapted to drive the signal-application electrode to apply the current, and, in combination, to drive the substance delivery unit to deliver the therapeutic substance. In an embodiment, the treatment unit includes a patient-alert unit, adapted to generate a patient-alert signal. In an embodiment, the treatment unit includes a substance delivery unit, adapted to deliver a therapeutic substance to the patient. In an embodiment, the substance delivery unit includes a pump. In an embodiment, the substance includes insulin, and the substance delivery unit is adapted to deliver the insulin to the patient. In an embodiment, the substance includes a drug, and the substance delivery unit is adapted to deliver the drug to the patient. In an embodiment, the drug is selected from the list consisting of: glyburide, glipizide, and chlorpropamide.

There is further provided, in accordance with a preferred embodiment of the present invention, apparatus for sensing electrical activity of a pancreas of a patient, including an electrode assembly, which includes:

one or more wire electrodes, each wire electrode including a curved portion, which curved portion is adapted to be brought in contact with the pancreas, and each wire electrode adapted to generate an activity signal indicative of electrical activity of pancreatic cells which are in a plurality of islets of the pancreas; and

a clip mount, to which the wire electrodes are fixed, which is adapted to secure the wire electrodes to the pancreas.

There is yet further provided, in accordance with a preferred embodiment of the present invention, apparatus for sensing electrical activity of a pancreas of a patient, including an electrode assembly, which includes:

a plurality of wire electrodes, adapted to be brought in contact with and to penetrate a surface of the pancreas, and to generate respective activity signals indicative of electrical activity of pancreatic cells which are in a plurality of islets of the pancreas; and

a mount, to which the wire electrodes are fixed, which is adapted to secure the wire electrodes to the pancreas.

There is still further provided, in accordance with a preferred embodiment of the present invention, apparatus for sensing electrical activity of a pancreas of a patient, including a patch assembly, which includes:

a patch, adapted to be coupled to tissue of the patient in a vicinity of the pancreas; and

one or more electrode assemblies, adapted to be coupled to the patch such that the electrode assemblies are in electrical contact with the tissue, and adapted to generate respective activity signals indicative of electrical activity of pancreatic cells which are in a plurality of islets of the pancreas.

In an embodiment, the apparatus includes a balloon, coupled to a surface of the patch not in contact with the tissue. In an embodiment, the apparatus includes a hydrogel, adapted to be applied to a surface of the patch not in contact with the tissue, so as to flexibly harden and maintain coupling of the patch to the tissue.

In an embodiment, the apparatus includes a sheet, coupled to a surface of the patch not in contact with the tissue, so as to protect the patch from motion of organs of the patient.

In an embodiment, the patch is adapted to have one or more sutures pass therethrough, to couple the patch to the tissue.

In an embodiment, the apparatus includes an adhesive, adapted to couple the patch to the tissue.

In an embodiment, the electrode assemblies include two electrode assemblies, adapted to facilitate a differential measurement of the electrical activity of the pancreas.

In an embodiment, each of the electrode assemblies includes:

a wire electrode; and

an insulating ring, surrounding the wire electrode.

In an embodiment, the patch includes one or more signal-processing components fixed thereto.

In an embodiment, at least one of the signal-processing components is selected; from the list consisting of: a preamplifier, a filter, an amplifier, an analog-to-digital converter, a preprocessor, and a transmitter.

In an embodiment, at least one of the signal-processing components is adapted to drive at least one of the electrode assemblies to apply a signal to a portion of the tissue, the signal configured so as to treat a condition of the patient.

In an embodiment, each of the electrode assemblies includes:

an inner wire electrode, adapted to function as a first pole of the electrode assembly;

an inner insulating ring, adapted to surround the inner wire electrode;

an outer ring electrode, adapted to surround the inner insulating ring, and to function as a second pole of the electrode assembly; and

an outer insulating ring, adapted to surround the outer ring electrode.

In an embodiment, the inner wire electrode is adapted to have a tissue-contact surface area approximately equal to a tissue-contact surface area of the outer ring electrode.

There is yet further provided, in accordance with a preferred embodiment of the present invention, apparatus including a patch, adapted to be implanted in contact with tissue of a patient, the tissue in a vicinity of a pancreas of the patient, the patch including one or more signal-processing components fixed thereto, which are adapted to process pancreatic electrical signals.

In an embodiment, at least one of the signal-processing components is selected from the list consisting of: a preamplifier, a filter, an amplifier, an analog-to-digital converter, a preprocessor, and a transmitter.

In an embodiment, the tissue includes tissue of the pancreas of the patient, and the patch is adapted to be coupled to the tissue of the pancreas.

In an embodiment, the tissue includes tissue of a duodenum of the patient, and the patch is adapted to be coupled to the tissue of the duodenum.

In an embodiment, the apparatus includes an electrode, adapted

to be coupled to tissue of the patient in a vicinity of the pancreas,

to generate an activity signal indicative of electrical activity of pancreatic cells which are in a plurality of islets of the pancreas, and

to be electrically coupled to at least one of the signal-processing components.

In an embodiment, at least one of the signal-processing components is adapted to drive the electrode to apply a signal to the pancreas, the signal configured so as to treat a condition of the patient.

There is also provided, in accordance with a preferred embodiment of the present invention, apparatus for sensing electrical activity of a pancreas of a patient, including:

a patch, adapted to be coupled to first tissue of the patient in a vicinity of the pancreas, the patch including a signal-processing component;

at least one electrode assembly, including:

an electrode, adapted to be coupled to second tissue of the patient in a vicinity of the pancreas and in a vicinity of the patch, and to generate an activity signal indicative of electrical activity of pancreatic cells which are in a plurality of islets of the pancreas; and

a wire having a first end and a second end, the first end physically and electrically coupled to the electrode, the second end including a surgical needle, adapted to be electrically coupled to the second end, the wire adapted to function as a suture for use with the needle, and the second end adapted to be physically and electrically coupled to the preamplifier.

In an embodiment, the signal-processing component includes a preamplifier.

In an embodiment, the second end is adapted to be physically and electrically coupled to the preamplifier by inserting the needle into the preamplifier.

In an embodiment, the needle is adapted to be broken after the wire is sutured to the second tissue, thereby leaving a broken portion of the needle fixed to the second end of the wire, and the second end of the wire is adapted to be physically and electrically coupled to the preamplifier by inserting the broken portion of the needle into the preamplifier.

There is additionally provided, in accordance with a preferred embodiment of the present invention, apparatus for sensing electrical activity of a pancreas of a patient, including an electrode, adapted to be coupled to tissue of the patient in a vicinity of the pancreas, and adapted to generate an activity signal indicative of electrical activity of pancreatic cells which are in a plurality of islets of the pancreas, the electrode including a hooking element, which includes a plurality of prongs, the prongs adapted to be collapsible while being inserted into the tissue, and to expand after insertion, thereby generally securing the electrode in the tissue.

There is yet additionally provided, in accordance with a preferred embodiment of the present invention, apparatus for sensing electrical activity of a pancreas of a patient, including an electrode, adapted to be coupled to tissue of the patient in a vicinity of the pancreas, and adapted to generate an activity signal indicative of electrical activity of pancreatic cells which are in a plurality of islets of the pancreas, the electrode including a spiral stopper element, adapted to secure the electrode in the tissue.

There is still additionally provided, in accordance with a preferred embodiment of the present invention, apparatus for sensing electrical activity of a pancreas of a patient, including an electrode, adapted to be coupled to tissue of the patient in a vicinity of the pancreas, and adapted to generate an activity signal indicative of electrical activity of pancreatic cells which are in a plurality of islets of the pancreas, the electrode including a corkscrew element, adapted to secure the electrode in the tissue.

There is further provided, in accordance with a preferred embodiment of the present invention, apparatus for sensing electrical activity of a pancreas of a patient, including an electrode assembly; including:

a connecting element;

an amplifier;

at least two wires, each wire having a proximal end and a distal end, the distal end of each wire adapted to be attached to the connecting element, and the proximal end of each wire adapted to be attached to the amplifier, each wire including an electrically-insulating coating attached thereto, adapted to cover a portion of the wire and to not cover at least one exposed site on the wire, so as to provide electrical contact between the exposed site and tissue of the pancreas; and

a suture, having a proximal end and a distal end, the proximal end adapted to be attached to the amplifier, and the distal end adapted to be connected to the connecting element.

In an embodiment, one of the exposed sites on a first one of the wires and one of the exposed sites on a second one of the wires are adapted to facilitate a differential measurement of the electrical activity of the pancreas.

In an embodiment, the apparatus includes a needle, attached to the distal end of the suture.

There is yet further provided, in accordance with a preferred embodiment of the present invention, apparatus for analyzing electrical activity of a pancreas of a patient, including:

a set of one or more electrodes, adapted to be coupled to the pancreas and to generate respective activity signals indicative of electrical activity of pancreatic cells; and

a control unit, adapted to:

receive the activity signals from the one or more electrodes,

analyze a frequency component of the received activity signals, and

generate an output signal responsive to the analysis.

There is still further provided, in accordance with a preferred embodiment of the present invention, apparatus for analyzing activity of a pancreas of a patient, including:

a set of one or more calcium electrodes, each of the calcium electrodes adapted to be coupled to the pancreas and to generate a signal indicative of a calcium level; and

a control unit, adapted to:

receive the signals from the one or more calcium electrodes,

analyze the received activity signals, and

generate an output signal responsive to the analysis.

In an embodiment, each of the electrodes is adapted to generate the signal indicative of an intracellular calcium level. In an embodiment, each of the electrodes is adapted to generate the signal indicative of an interstitial calcium level.

There is also provided, in accordance with a preferred embodiment of the present invention, a method for sensing electrical activity of a pancreas of a patient, including:

sensing electrical activity of pancreatic cells which are in a plurality of islets of the pancreas;

generating activity signals responsive thereto;

receiving the activity signals;

analyzing the activity signals; and

generating an output signal responsive to the analysis.

There is additionally provided, in accordance with a preferred embodiment of the present invention, a method for sensing electrical activity of a pancreas of a patient, including:

sensing, at each of one or more sites of the pancreas, electrical activity of pancreatic cells in a respective plurality of islets;

generating activity signals responsive thereto;

receiving the activity signals;

analyzing the activity signals; and

generating an output signal responsive to the analysis.

There is yet additionally provided, in accordance with a preferred embodiment of the present invention, a method for monitoring a blood glucose level of a patient, including:

sensing spontaneous electrical activity of pancreatic cells;

generating activity signals responsive thereto;

receiving the activity signals;

analyzing the activity signals so as to determine a change in the glucose level; and

generating an output signal responsive to determining the change.

There is still additionally provided, in accordance with a preferred embodiment of the present invention, a method for monitoring a blood insulin level of a patient, including:

sensing spontaneous electrical activity of pancreatic cells;

generating activity signals responsive thereto;

receiving the activity signals;

analyzing the activity signals so as to determine a change in the insulin level; and

generating an output signal responsive to determining the change.

There is also provided, in accordance with a preferred embodiment of the present invention, a method for analyzing electrical activity of a pancreas of a patient, including:

sensing electrical activity at one or more pancreatic sites;

generating activity signals responsive thereto;

receiving the activity signals;

analyzing the activity signals so as to identify an aspect thereof which is indicative of activity of pancreatic alpha cells; and

generating an output signal responsive to identifying the aspect.

There is further provided, in accordance with a preferred embodiment of the present invention, a method for analyzing electrical activity of a pancreas of a patient, including:

sensing electrical activity at one or more pancreatic sites;

generating activity signals responsive thereto;

receiving the activity signals;

analyzing the activity signals so as to identify an aspect thereof which is indicative of activity of pancreatic beta cells; and

generating an output signal responsive to identifying the aspect.

There is still further provided, in accordance with a preferred embodiment of the present invention, a method for analyzing electrical activity of a pancreas of a patient, including:

sensing electrical activity at one or more pancreatic sites;

generating activity signals responsive thereto;

receiving the activity signals;

analyzing the activity signals so as to identify an aspect thereof which is indicative of activity of pancreatic delta cells; and

generating an output signal responsive to identifying the aspect.

There is yet further provided, in accordance with a preferred embodiment of the present invention, a method for analyzing electrical activity of a pancreas of a patient, including:

sensing electrical activity at one or more pancreatic sites;

generating activity signals responsive thereto;

receiving the activity signals;

analyzing the activity signals so as to identify an aspect thereof which is indicative of activity of polypeptide cells; and

generating an output signal responsive to identifying the aspect.

There is also provided, in accordance with a preferred embodiment of the present invention, a method for coupling an electrode to a pancreas of a patient, including:

peeling back a portion of connective tissue surrounding the pancreas, so as to create a pocket;

inserting the electrode into the pocket; and

suturing the electrode to the connective tissue.

There is additionally provided, in accordance with a preferred embodiment of the present invention, a method for sensing electrical activity of a pancreas of a patient, including:

sensing, at each of one or more sites of the pancreas, electrical activity of pancreatic cells;

generating activity signals responsive thereto;

receiving the activity signals;

analyzing a frequency component of the activity signals; and

generating an output signal responsive to the analysis.

There is yet additionally provided, in accordance with a preferred embodiment of the present invention, a method for sensing activity of a pancreas of a patient, including:

sensing, at each of one or more sites of the pancreas, a calcium level;

generating activity signals responsive thereto;

receiving the activity signals;

analyzing the activity signals; and

generating an output signal responsive to the analysis.

The present invention will be more fully understood from the following detailed description of the preferred embodiments thereof, taken together with the drawings, in which:

### BRIEF DESCRIPTION OF THE DRAWINGS

FIG. 1A is a schematic illustration of the external surface of a pancreas, showing the placement of electrodes thereon, in accordance with a preferred embodiment of the present invention;

FIG. 1B is a schematic block diagram of a control unit, which receives signals from the electrodes shown in FIG. 1A, in accordance with a preferred embodiment of the present invention;

FIGS. 2A, 2B and 2C are schematic illustrations of electrodes for sensing activity of the pancreas, in accordance with respective preferred embodiments of the present invention;

FIG. 3A is a schematic illustration of a two-electrode patch assembly, in accordance with a preferred embodiment of the present invention;

FIG. 3B is a schematic illustration of a concentric electrode patch assembly, in accordance with a preferred embodiment of the present invention;

FIG. 3C is a schematic top-view illustration of two button electrode assemblies attached to a preamplifier, in accordance with a preferred embodiment of the present invention;

FIG. 3D is a schematic cross-sectional side-view illustration of one of the button electrode assemblies of FIG. 3C, in accordance with a preferred embodiment of the present invention;

FIG. 3E is a schematic perspective illustration of a single electrode assembly, in accordance with a preferred embodiment of the present invention;

FIG. 3F is a schematic illustration of a hooking element of an electrode, in accordance with a preferred embodiment of the present invention;

FIG. 3G is a schematic illustration of another hooking element of an electrode, in accordance with a preferred embodiment of the present invention;

FIG. 3H is a schematic illustration of a corkscrew electrode, in accordance with a preferred embodiment of the present invention;

FIG. 3I is a schematic illustration of an electrode assembly, in accordance with a preferred embodiment of the present invention;

FIG. 4 is a schematic block diagram of a signal-processing patch assembly, in accordance with a preferred embodiment of the present invention;

FIGS. 5A, 5B, 5C, 6A, 6B, 6C, 7A, 7B, 7C, 8A, 8B, 8C, 9A, 9B, 10A, and 10B are graphs showing measurements or analysis of electrical activity taken during experiments performed in accordance with a preferred embodiment of the present invention;

FIGS. 11, 12, and 13 show the results of signal processing of the experimental results shown in FIGS. 9A and 9B, in accordance with a preferred embodiment of the present invention;

FIG. 14 shows the results of signal processing of experiments performed on dogs, in accordance with a preferred embodiment of the present invention;

FIG. 15 shows the results of electrical activity measurements made in the gastrointestinal tract and in the pancreas of a dog, during experiments performed in accordance with a preferred embodiment of the present invention;

FIG. 16 shows additional measurements of pancreatic and GI tract electrical activity, during experiments on a dog performed in accordance with a preferred embodiment of the present invention;

FIG. 17 shows measurements of pancreatic electrical activity, during experiments on a dog performed in accordance with a preferred embodiment of the present invention;

FIG. 18 shows electrode apparatus for measuring pancreatic electrical activity, in accordance with a preferred embodiment of the present invention; and

FIGS. 19-47 show experimental data recorded in accordance with preferred embodiments of the present invention.

### DETAILED DESCRIPTION OF PREFERRED EMBODIMENTS

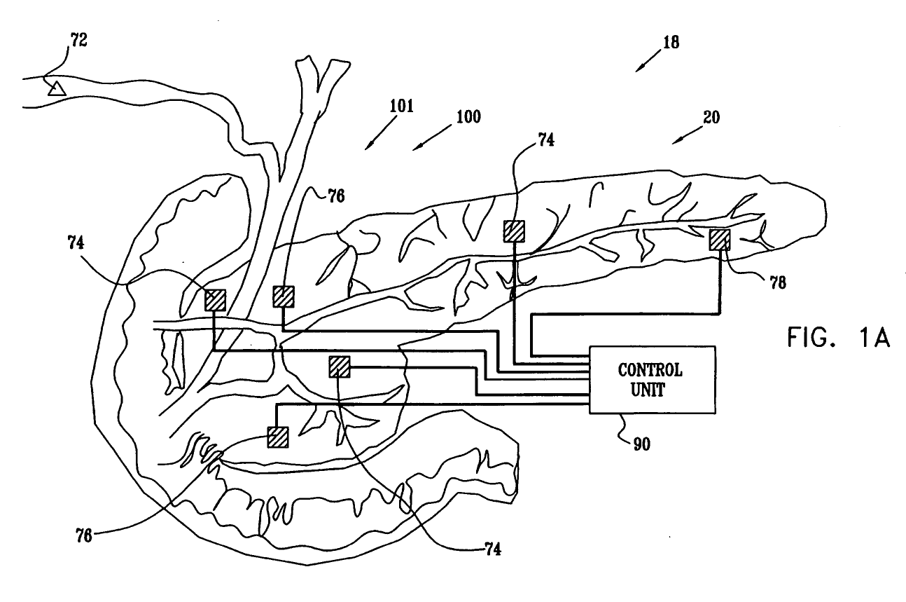

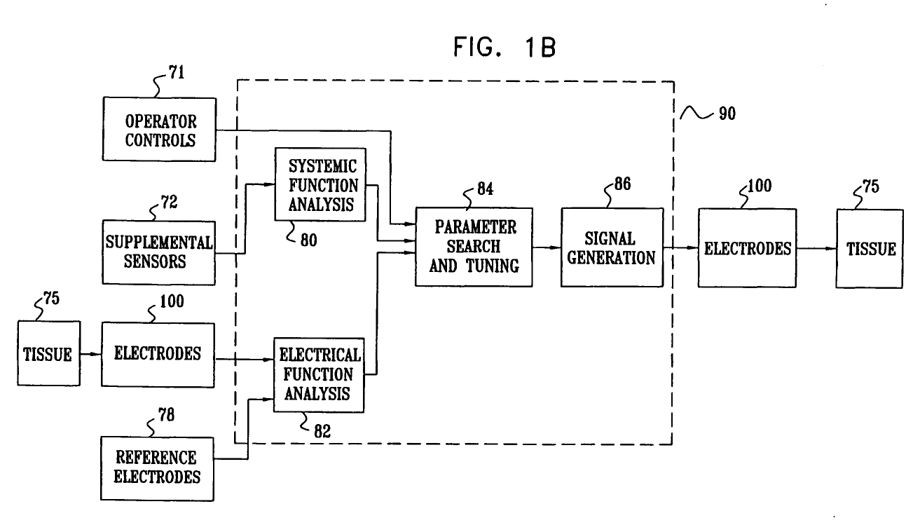

FIG. 1A is a schematic illustration of apparatus 18, which senses electrical activity of a pancreas 20 of a patient, in accordance with a preferred embodiment of the present invention. Apparatus 18 preferably comprises an implantable or external control unit 90, which is electrically coupled to electrodes 100 and/or supplemental sensors 72, which sense, for example, blood sugar, SvO2, pH, pCO2, pO2, blood insulin levels, blood ketone levels, ketone levels in expired air, blood pressure, respiration rate, respiration depth, a metabolic indicator (e.g., NADH), or heart rate. Electrodes 100 are preferably located in, on, or in a vicinity of the pancreas. Appropriate sites for electrodes 100 include, but are not limited to, on a surface tissue of or in pancreas 20 (such as on or in the head, body, or tail of the pancreas), in or near a blood vessel in a vicinity of pancreas 20 (such as a blood vessel of the pancreas). Supplemental sensors 72 are preferably located on the pancreas or elsewhere in and/or on the body of the patient, and are configured to generate supplemental signals. Appropriate sites for supplemental sensors 72 include, but are not limited to, the duodenum and the stomach, as well as those sites described above as appropriate for electrodes 100. For some applications, supplemental sensors 72 comprise an accelerometer, for detecting stomach, duodenum, and/or respiratory movements. Electrodes 100 are electrically coupled with control unit 90 over leads or wirelessly, such as by using induction coils, coupling capacitive signal transferors, near-field electromagnetic transmission, radiofrequency transmission, or other wireless transmission techniques known in the art.

In a preferred embodiment, recorded electrical activity signals detected by electrodes 100 are amplified and transferred by wires out of the patient's body and/or transferred to a signal-receiving device which interacts with a device that produces a therapy (e.g., modulating insulin secretion).

For some applications in which communication with an external unit is desired, in order to avoid long wires and skin crossing, wireless transmission is used. For example, transmission may be in the ISM frequency band, typically in frequencies of 13-27 MHz. Since the transmission utilized is typically for short distances, e.g., tens of centimeters, working in the low frequencies is preferably accomplished by means of the magnetic field produced by a loop antenna. More than one loop (e.g., mutually-perpendicular loops, or loops at another angular offset) are used in some applications. The transmission method can be analog, e.g., by amplitude modulation (AM) or by frequency modulation (FM), or it may be digital, as described hereinbelow. For digital transmission, the signal is sampled (preferably after suitable filtering), and then transmitted.

On-Off keying (OOK) is a preferred digital transmission method. Alternatively, other digital transmission methods known in the art are used, such as frequency shift keying (FSK) or phase shift keying (PSK, BPSK, QPSK).

In a preferred OOK embodiment, the output of a serial analog-to-digital converter is input into a resonator, which may resonate, for example, by the interaction between a coil and a capacitor, or by means of a SAW-based resonator or other circuit known in the art, connected to the coil.

In order to reduce power consumption for the data transmission, it is possible to avoid active transmission at the pancreatic site, and instead use an externally-driven magnetic field. In this case, the internal unit on the pancreas preferably includes a switched coil. The coil is either connected or disconnected according to the data bits to be transmitted to the external unit. Switching of the coil may be accomplished with FET's or any suitable technique known in the art.

The switching of the switching coil at the pancreas is detected by the external unit (outside of the patient's body) as slight pulses in the current consumption of the external coil, due to the changes in the coupling between the external coil and the internal switching coil. (Changing the load is detectable as transient current changes in the external emitter coil.)

Pre-processing of the recorded data is preferably performed prior to transmission to the external unit. For example, the data may be analyzed, and the data stream compressed and/or encoded, such as with error-correcting codes, e.g., repetitions, convolutions, and interleaves.

For some applications, in order to further reduce power consumption by the internal circuitry coupled to the pancreas, the energy source for all of the circuitry (e.g., amplifiers, filters, A/D, pre-processing, transmission, therapy application, etc.) is based on induction. In this method, an externally-driven magnetic field transfers energy into the circuit. Low frequencies (e.g., a few KHz) are typically used, although other frequencies can be used as well.

In the internal unit, the energy is received by a coil which resonates at the transmitted frequency. The received signal is preferably converted into DC, filtered and regulated. For some applications, this energy charges an internal energy source (e.g., a battery or capacitor). For other applications, the energy directly supplies the operation of the internal circuitry.

In an embodiment, most of the internal circuitry is implemented in a single chip, with direct links to only a few off-chip components, such as electrodes 100 and coils. Preferably, the chip performs signal amplification, conditioning, sampling, analysis, encoding, and modulation, and switches the switching coil to pass information to the external unit.

Alternatively or additionally, the internal unit wirelessly receives commands from the external unit, using the techniques described herein (e.g., OOK) or others known in the art. For example, these commands may include: turn on/off, change gain, and change filter parameters.

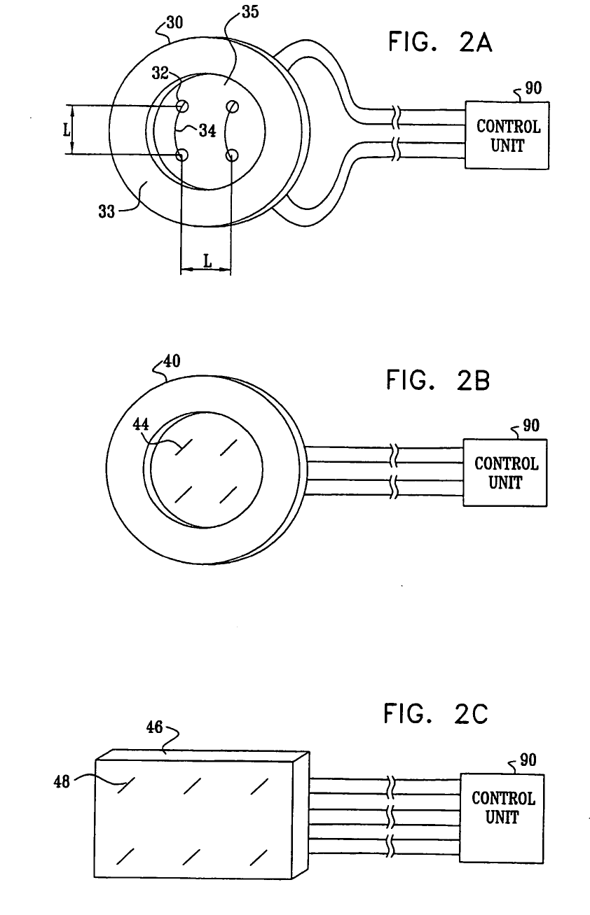

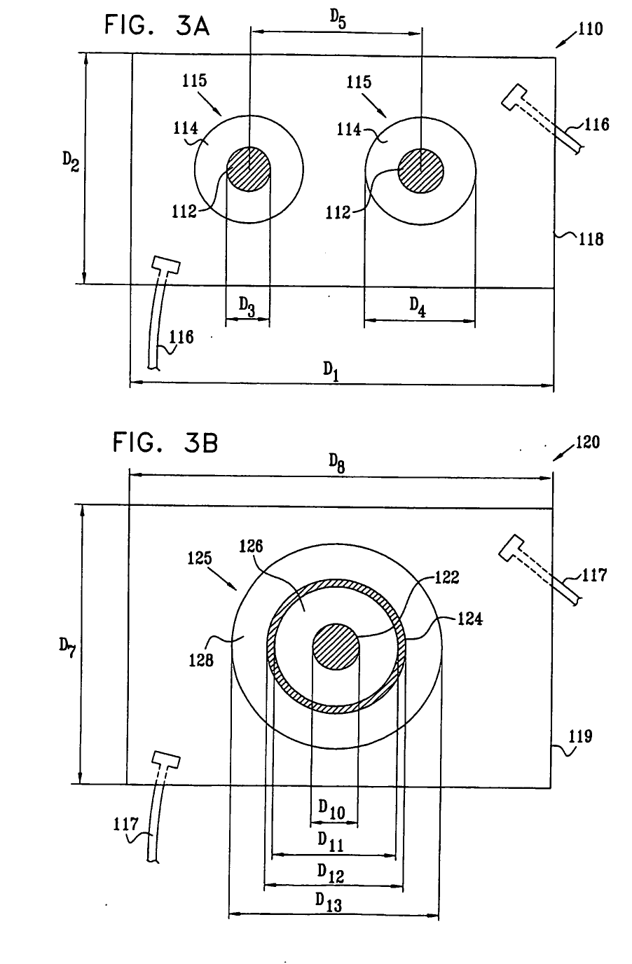

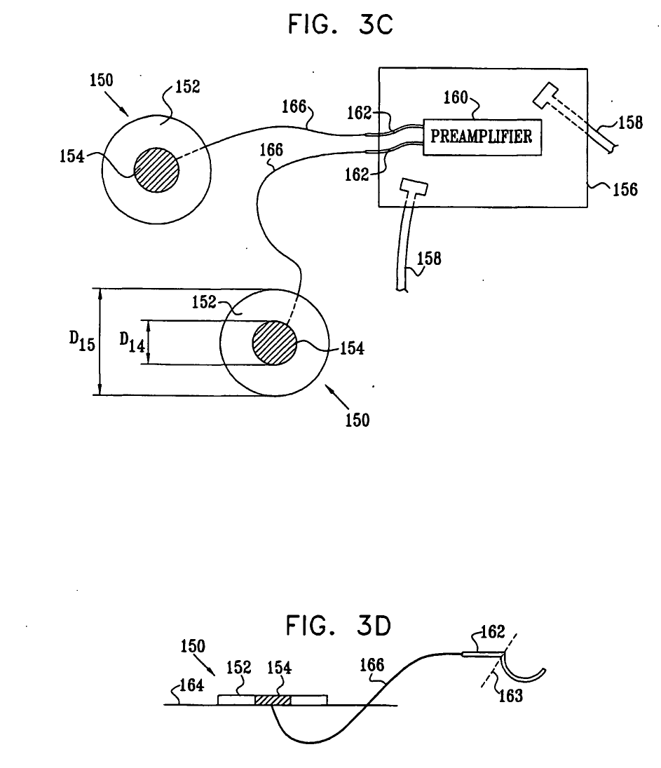

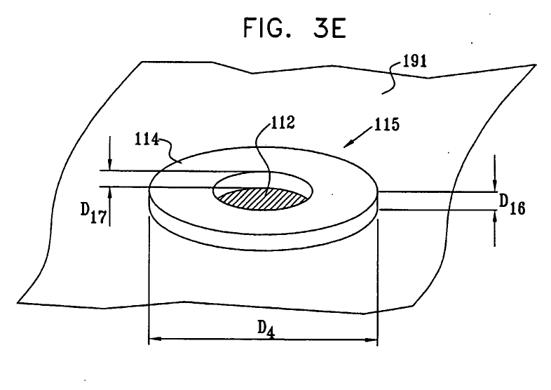

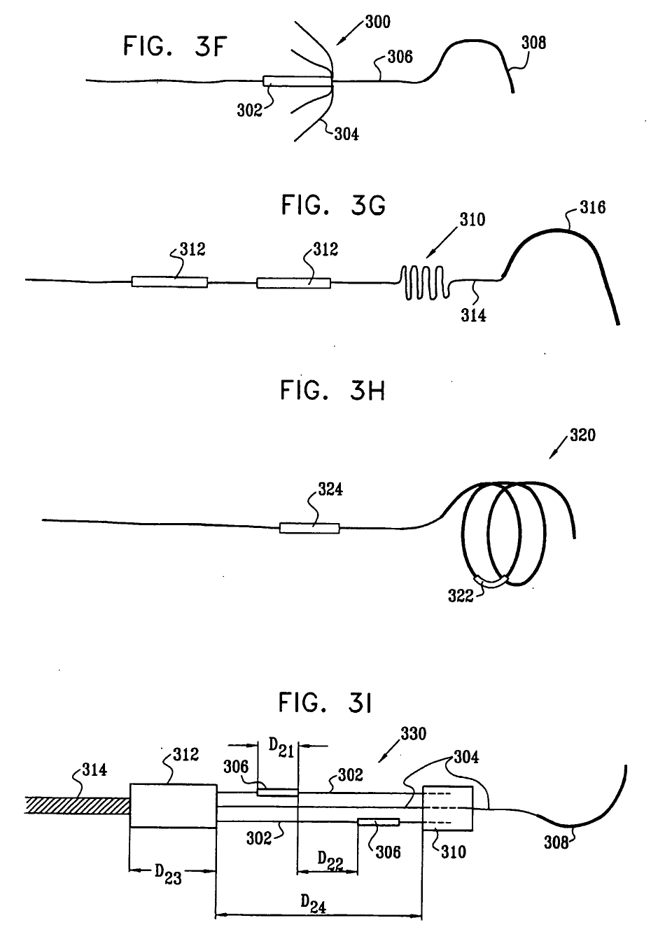

Electrodes 100 comprise one or more of the following: (a) local sense electrodes 74, configured to sense electrical activity of pancreas 20 and generate activity signals responsive to the electrical activity, (b) signal application electrodes 76, configured to apply signal-applications to pancreas 20, (c) electrodes configured to function both as local sense electrodes and signal application electrodes, and generate respective activity and signal-applications, and/or (d) a combination of (a), (b) and (c). Electrodes 100 preferably comprise one or more of the electrodes described hereinbelow with reference to FIGS. 2A, 2B, 2C, 3A, 3B, 3C and/or 3D. Alternatively or additionally, electrodes 100 comprise substantially any suitable electrode known in the art of electrical stimulation or sensing in tissue, such as those designed for recording electrical activity in the brain. It is to be understood that the placement and number of electrodes and sensors in FIG. 1A are shown by way of example only.

In a preferred embodiment of the present invention, in response to receiving and analyzing activity signals and/or supplemental signals, generated by electrodes 100 and/or supplemental sensors 72, respectively, control unit 90 applies a treatment by means of a treatment unit 101, comprising, for example, one or more of electrodes 100, which are driven by the control unit to apply signal-applications to at least a portion of pancreas 20. Alternatively or additionally, treatment unit 101 may comprise other apparatus known in the art (not shown), including, but not limited to:
an external or implanted pump for delivering a drug or chemical to the patient, such as insulin or therapeutic agents that alter blood glucose levels, such as “DIA BETA” (glyburide; Hoechst-Roussel), “GLOCONTROL” (glipizide; Pfizer) and “DIABINESE” (chlorpropamide; Pfizer); and/or
a patient-alert unit, that generates a signal instructing the patient to take an action, such as self-administering a drug or chemical, such as insulin, or eating. For some applications, the patient-alert unit comprises a display, in which case the signal is a visual signal; alternatively or additionally, the signal is an audible tone or tactile signal, such as a vibration signal.

For some applications, a pump delivers, and/or a patient-alert unit instructs the patient to self-administer, a drug that blocks glucagon, the production of which may be stimulated by signals applied by electrodes 100 functioning as treatment unit 101. When treatment unit 101 comprises one or more of electrodes 100, control unit 90 preferably modifies the signal-applications applied through the electrodes responsive to signals from supplemental sensors 72 and/or activity signals generated by electrodes 100 functioning as local sense electrodes, as described hereinbelow. Alternatively or additionally, apparatus 18 is configured to operate in a diagnostic mode, and electrical measurements made by the apparatus are stored for later analysis, such as by a physician or by an automated analysis system, such as a computer system. For some applications, control unit 90 applies the treatment with respect to a time that a patient commences eating, e.g., 10 minutes before eating, during eating, or 10 minutes after commencement of eating.

Typically, electrodes 100 convey activity signals to control unit 90 responsive to spontaneous electrical activity of the pancreas, e.g., activity which occurs in the course of natural, ongoing processes of the pancreas. For some applications, however, a synchronizing signal is first applied (e.g., using techniques described in the above-cited U.S. Pat. Nos. 5,919,216, 6,093,167 and/or 6,261,280 to Houben et al.), and pancreatic electrical activity is measured subsequent thereto. Preferably, the synchronizing signal is applied by one or more of electrodes 100.

In a preferred embodiment, one or more reference electrodes 78 are placed near the pancreas or elsewhere in or on the patient's body. Optionally, at least one of electrodes 78 comprises a metal case of control unit 90. In some applications, the reference electrodes are used to reduce any effects of artifacts on recording pancreatic electrical activity, which may arise due to respiratory movements, neural activity, cardiac electrical phenomena, electromyographic phenomena, smooth muscle electrical activity, and/or gastrointestinal tract electrical phenomena.

For applications in which control unit 90 applies signal-application signals to the pancreas, methods and techniques are preferably employed which are described in one or more of the following applications/publications cited hereinabove: (a) U.S. Provisional Patent Application 60/123,532, filed Mar. 5, 1999, entitled “Modulation of insulin secretion,” (b) PCT Publication WO 00/53257 to Darwish et al., and the corresponding U.S. patent application Ser. No. 09/914,889, filed Jan. 24, 2002, or (c) PCT Publication WO 01/66183 to Darvish et al.

In an embodiment, the signal-application signals are synchronized with respect to a phase or state of the pancreas. For example, the signal-application signals may be applied with respect to a phase in a metabolic and/or insulin oscillation. NADH is a metabolic indicator suitable for facilitating this approach. Alternatively or additionally, insulin oscillations measured using techniques described herein are used to coordinate the timing of application of the signal-application signals. Depending on application, the signal-application signals may be applied during high- or low-points in the measured insulin oscillations. Further alternatively or additionally, signal-application signals are timed with respect to the beginning, middle, or end of a recorded burst or group of bursts. Still further alternatively or additionally, the signal-application signals are applied during an inter-burst period.

FIG. 1B is a schematic block diagram of control unit 90, in accordance with a preferred embodiment of the present invention. One or more of electrodes 100 functioning as local sense electrodes are preferably coupled to provide activity signals to an electrical function analysis block 82 of control unit 90. The activity signals preferably provide information about various aspects of the electrical activity of the pancreas to block 82, which analyzes the signals and, optionally, actuates control unit 90 to initiate or modify electrical energy applied to the pancreas responsive to the analysis, preferably using one or more of electrodes 100. Alternatively or additionally, other responses to the measurements are implemented, such as those described hereinabove with reference to treatment unit 101. Preferably, signals applied to the pancreas are adjusted by the control unit, responsive to the activity signals, in order to yield a desired response, e.g., a change in a predetermined pancreatic electrical profile. Examples of changes in such a profile include a change in amplitude, energy, rate, frequency of bursts, frequency within a single burst, amplitude of a frequency component while another component remains generally constant, glucose level, and output of one of supplemental sensors 72.

Preferably, block 82 conveys results of its analysis to a “parameter search and tuning” block 84 of control unit 90, which iteratively modifies characteristics of the electrical signals applied to the pancreas in order to attain a desired response. Further preferably, operating parameters of block 84 are entered during an initial calibration period by a human operator of the control unit using operator controls 71, which comprise an input unit, comprising, for example, a keyboard, a keypad, one or more buttons, and/or a mouse. Block 84 typically utilizes multivariate optimization and control methods known in the art in order to cause one or more electrical parameters (e.g., burst magnitude, amplitude of different burst spectral components, and/or burst rate or duration), chemical parameters (e.g., glucose or insulin values) and/or other measured parameters to converge to desired values.

In general, each one of electrodes 100, when functioning as a signal application electrode, may convey a particular waveform to pancreas 20, differing in certain aspects from the waveforms applied by the other electrodes. The particular waveform to be applied by each electrode is determined by control unit 90, initially under the control of the operator. Aspects of the waveforms which are set by the control unit, and may differ from electrode to electrode, typically include parameters such as time shifts between application of waveforms at different electrodes, waveform shapes, amplitudes, DC offsets, durations, and duty cycles. For example, the waveforms applied to some or all of electrodes 100 may comprise a monophasic square wave pulse, a sinusoid, a series of biphasic square waves, or a waveform including an exponentially-varying characteristic. Generally, the shape, magnitude, and timing of the waveforms are optimized for each patient and for each electrode, using suitable optimization algorithms as are known in the art. For example, one electrode may be driven to apply a signal, while a second electrode on the pancreas is not applying a signal. Subsequently, the electrodes may change functions, whereby the second electrode applies a signal, while the first electrode is not applying a signal.

For the purposes of these embodiments of the present invention, block 84 typically modifies a set of controllable parameters of the signal-application signals, responsive to the measured parameters, in accordance with values in a look-up table and/or pre-programmed formulae stored in an electronic memory of control unit 90. The controllable parameters may comprise, for example, pulse timing, magnitude, offset, monophasic or biphasic shape, applied signal frequency, and pulse width. In a preferred embodiment, signal-application signals are applied in biphasic rectangular pulses, having pulse widths of: (a) between about 2 and about 100 ms, most preferably about 5 ms, in the positive phase, and (b) between about 2 and about 100 ms, most preferably about 5 ms, in its negative phase, and having a frequency of between about 5 and about 100 Hz, most preferably 5 Hz, 20 Hz or 100 Hz. In this embodiment, the signals are applied either as single pulses, or in a burst with a duration preferably between about 500 ms and about several seconds. Preferably, the application of the signals is repeated approximately every 1-10 minutes. Preferably, the controllable parameters are conveyed by block 84 to a signal generation block 86 of control unit 90, which generates, responsive to the parameters, electrical signal-application signals that are applied by electrodes 100, when functioning as signal application electrodes, to pancreas 20. Block 86 preferably comprises amplifiers, isolation units, and other standard circuitry known in the art of electrical signal generation. It is to be understood that although the components of control unit 90 are shown in the figures as incorporated in an integrated unit, this is for the sake of illustration only. In some embodiments of the present invention, one or more of the components of control unit 90 are located in one or more separate units, for example implantable patches, as described hereinbelow, coupled to one another and/or control unit 90 over wires or wirelessly.

FIG. 2A is a schematic illustration of one portion of a clip mount 30 for application of one or more wire electrodes 34 to the surface of pancreas 20, in accordance with a preferred embodiment of the present invention. For some applications, one or more of electrodes 100 comprise wire electrodes 34 fixed to clip mount 30. Clip mount 30 preferably comprises an inner non-conducting region 35 and an outer non-conducting border 33. Region 35 and border 33 preferably comprise silicone, Parylene, Teflon, polyamide, and/or glass. For some applications, one of region 35 and border 33 is non-flexible, while the other is flexible. Alternatively, region 35 and border 33 comprise the same material, and/or are an integrated unit (e.g., shaped as a generally flat disk).

In the preferred embodiment shown in FIG. 2A, each of two wire electrodes 34 is looped through two holes 32 of clip mount 30, so that the curved portion of the wire electrode is exposed to the surface of the pancreas. Preferably, the four holes 32 are arranged in a square, with the length L of each side between about 1 and about 10 mm, most preferably 4 mm. In other preferred embodiments, a single wire electrode 34 or more than two wire electrodes 34 are provided. In a preferred embodiment, a one-piece clip mount having spring-like properties is used to secure one or more electrodes to the pancreas.

FIGS. 2B and 2C are schematic illustrations of respective mounts 40 and 46 for application of respective tissue-penetrating electrodes 44 and 48 to pancreas 20, in accordance with preferred embodiments of the present invention. For some applications, one or more of electrodes 100 comprise electrodes 44 and/or 48 fixed to mounts 40 and 46, respectively. Preferably, the tissue-penetrating electrodes comprise needles or wires. Mount 40 is generally similar to clip mount 30, except for the type of electrodes.

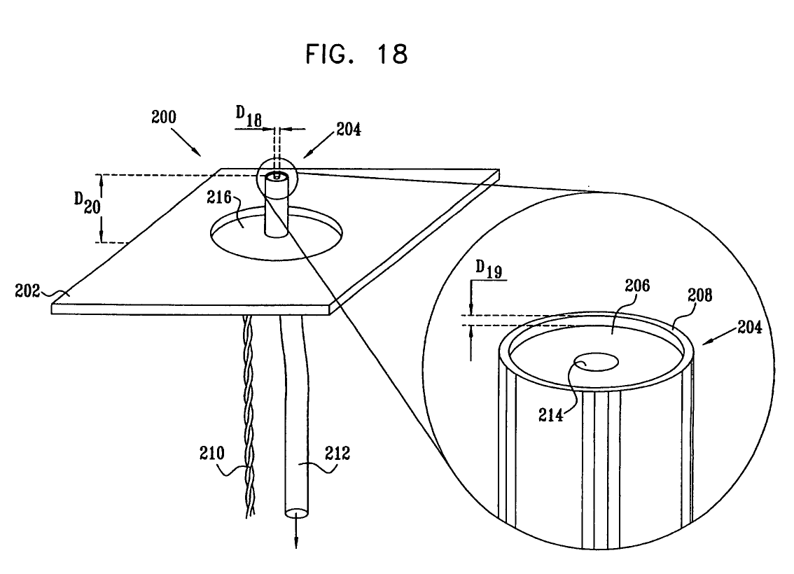

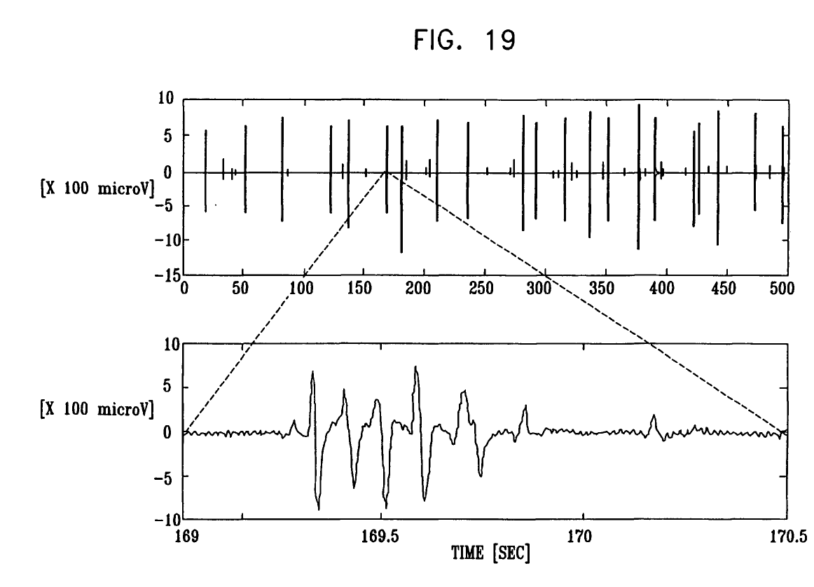

FIG. 3A is a schematic illustration of a two-electrode patch assembly 110, for use in some applications, in accordance with a preferred embodiment of the present invention. Patch assembly 110 preferably comprises a patch 118, preferably made of silicone, Parylene, polyamide, or another flexible biocompatible material, and two monopolar electrode assemblies 115. For some applications, at least one set of two electrodes 100 comprises two electrode assemblies 115 coupled to patch assembly 110. Each monopolar electrode assembly 115 preferably comprises a wire electrode 112 surrounded by an insulating ring 114, such as a glass, silicone or polyamide ring. Wire electrode 112 is exposed on one side of patch 118, and leads coupled to electrode 112 exit electrode assembly 115 towards the other side of the patch (leads not shown). Patch 118 is coupled to tissue of the patient, such as tissue of the pancreas, preferably by suturing using sutures 116 which emerge from the patch. Although two such sutures are shown in FIG. 3A, this is for clarity of illustration only; actual patches can have one suture or more than two sutures. Advantageously, suturing with the sutures generally results in a good connection between the exposed portion of wire electrode 112 and the tissue. Alternatively or additionally, patch 118 is coupled to tissue of the patient with a biocompatible adhesive such as biological glue (Quixil, Omrix Bio-pharmaceuticals, Rehovot, Israel). For some applications, a cavity, generally similar to cavity 216 described hereinbelow with reference to FIG. 18, disposed around electrode assembly 115, allows any excess biological glue which may have been applied to the patch to collect around the insulating material, without contaminating the electrode itself.

Wire electrodes 112 preferably comprise a biocompatible material, such as platinum/iridium (Pt/Ir), titanium, titanium nitride, or MP35N. The length D₁ and width D₂ of patch 118 are preferably between about 2 mm and about 20 mm, and between about 2 mm and about 10 mm, respectively. Most preferably, D₁ equals 4 mm and D₂ equals 1.2 cm. Preferably, the diameter D₃ of wire electrodes 112 is between about 0.5 mm and about 5 mm, most preferably 0.7 mm, and the diameter D₄ of insulating rings 114 is between about 0.5 mm and about 5 mm, most preferably 1.6 mm. When the electrode assemblies are of these dimensions, the distance D₅ between the centers of the electrode assemblies is preferably between about 2 and about 10 mm, most preferably 4 mm.

Reference is made to FIG. 3E, which is a schematic perspective illustration of a single electrode assembly 115 fixed to a portion 191 of patch 118, in accordance with a preferred embodiment of the present invention. Preferably, insulating ring 114 protrudes from the top surface of portion 191 by a distance D₁₆ of between about 0.5 mm and about 2.0 mm, most preferably about 1.5 mm. Preferably, wire electrode 112 is recessed in insulating ring 114 by a distance D₁₇ of between about 0.5 mm and about 2.0 mm, most preferably about 0.7 mm.

FIG. 3B is a schematic illustration of a concentric electrode patch assembly 120, for use in some applications, in accordance with a preferred embodiment of the present invention. Patch assembly 120 preferably comprises a patch 119, preferably made of silicone, polyamide, or another flexible biocompatible material, and a single bipolar concentric electrode assembly 125. For some applications, at least one of electrodes 100 comprises electrode assembly 125 fixed to patch 119. Concentric electrode assembly 125 comprises an inner wire electrode 122 and an outer ring electrode 124, with an inner insulating ring 126, such as a glass, silicone or polyamide ring, separating inner wire electrode 122 and outer ring electrode 124. Concentric electrode assembly 125 preferably also comprises an outer insulating ring 128, such as a glass, silicone or polyamide ring, surrounding outer ring electrode 124. Preferably, but not necessarily, the surface areas of the inner wire electrode and outer ring electrode are substantially equal. Inner wire electrode 122 and outer ring electrode 124 are exposed on one side of patch 119, and leads coupled to electrodes 122 and 124 exit concentric electrode assembly 125 towards the other side of the patch (leads not shown). Patch 119 is coupled to tissue of the patient, such as tissue of the pancreas, preferably by suturing using sutures 117 which emerge from the patch. Although two sutures are shown in FIG. 3B, this is for clarity of illustration only; actual patches can have one suture or more than two sutures. Advantageously, suturing with the sutures generally results in a good connection between the exposed portion of the electrodes and the tissue. Alternatively or additionally, patch 118 is coupled to tissue of the patient with a biocompatible adhesive such as biological glue (Quixil, Omrix Bio-pharmaceuticals, Rehovot, Israel). For some applications, a cavity, generally similar to cavity 216 described hereinbelow with reference to FIG. 18, disposed around electrode assembly 115, allows any excess biological glue which may have been applied to the patch to collect around the insulating material, without contaminating the electrode itself.

The electrodes preferably comprise a biocompatible material, such as platinum/iridium (Pt/Ir), titanium, titanium nitride or MP35N. The width D₇ and length D₈ of patch 119 are preferably between about 2 mm and about 10 mm, and between about 2 mm and about 20 mm, respectively. Most preferably, patch 119 is generally square, and D₇ and D₈ each equal about 7 mm. Preferably, (a) the diameter D₁₀ of inner wire electrode 122 is between about 0.5 mm and 5 mm, most preferably 1.2 mm, (b) the inner diameter D₁₁ of outer ring electrode 124 is between about 1 mm and about 5 mm, most preferably 3.1 mm, (c) the outer diameter D₁₂ of outer ring electrode 124 is between about 1 mm and about 10 mm, most preferably 3.2 mm, such that D₁₂−D₁₁ is typically between 0.1 mm and 0.5 mm, and (d) the diameter D₁₃ of outer insulating ring 128 is between about 1 mm and about 10 mm, most preferably 3.8 mm. Preferably, insulating rings 126 and 128 protrude from the top surface of patch 119 by a distance of between about 0.5 mm and about 2.0 mm, most preferably about 1.5 mm. Preferably, inner wire electrode 122 and outer ring electrode 124 are recessed in the insulating rings by a distance of between about 0.5 mm and about 2.0 mm, most preferably about 1.5 mm. (These latter dimensions can best be seen in FIG. 3E, described hereinabove with reference to electrode assembly 115.)

FIG. 3C is a schematic top-view illustration of two button electrode assemblies 150 attached to a preamplifier 160, in accordance with a preferred embodiment of the present invention. Each button electrode assembly 150 comprises an electrode 154 surrounded by an insulating ring 152, such as a glass, silicone or polyamide ring, and an electrically-insulated wire 166. One end of the wire is connected to electrode 150, preferably in the vicinity of the center of the electrode, and the other end of the wire comprises a needle 162 or other connector. Electrodes 154 preferably comprise a biocompatible material, such as platinum/iridium (Pt/Ir), titanium, titanium nitride, or MP35N. Preferably, the diameter D₁₄ of electrodes 154 is between about 0.5 mm and about 5 mm, most preferably 0.7 mm, and the diameter D₁₅ of insulating rings 152 is between about 0.5 mm and about 5 mm, most preferably 1.6 mm. Electrode 154 is preferably flush with insulating ring 152, as seen in FIG. 3D.

Reference is now made to FIG. 3D, which is a schematic cross-sectional side-view illustration of one of button electrode assemblies 150, in accordance with a preferred embodiment of the present invention. Needle 162 is used to suture electrode 150 to surface tissue 164 of a pancreas. After the suturing has been completed, needle 162 is preferably broken approximately along line 163. The remaining portion of the needle is inserted, preferably by force, into preamplifier 160 (FIG. 3C), which is attached to a patch 156, preferably made of silicone, polyamide, or another flexible biocompatible material. Patch 156 is then coupled to tissue 164, at a distance (e.g., about 1 cm to about 10 cm) selected so as to keep wire 166 moderately slack, thereby avoiding disturbing of the electrode during movement of the tissue. Alternatively, patch 156 is sutured to tissue 164 prior to the insertion of needle 162 into preamplifier 160. Patch 158 is preferably coupled to tissue 164 by suturing, using sutures 158, and/or by the use of biological glue.

Preferably, in order to improve the attachment and contact of the electrodes described hereinabove to tissue of the patient, a hydrogel is applied on top of the patch or mount containing the electrodes, and/or around this patch (e.g., 1-10 mm from the edge of the patch or mount), so as to flexibly harden and maintain the mechanical coupling of the patch or mount to the pancreas and/or act as a shock absorber, protecting the patch or mount during contact with or motion of organs of the subject, such as the stomach. Alternatively or additionally, a balloon filled with a gas, such as CO₂, or a liquid, such as saline solution, is placed on the top surface of the patch or mount, so as to act as a shock absorber, protecting the patch or mount during contact with or motion of organs of the subject, such as the stomach. Further alternatively or additionally, in order to reduce the likelihood that organs near the electrodes catch on the top of the electrodes, a sheet made of Teflon® or other similar material is attached to the top of the electrode patch or mount. Thus, organs near the electrode move smoothly against this sheet.

FIG. 3F is a schematic illustration of a hooking element 300 of an electrode 302, in accordance with a preferred embodiment of the present invention. For some applications, one or more of electrodes 100, such as the electrodes of two-electrode patch assembly 110 (FIG. 3A), single-electrode patch assembly 120 (FIG. 3B), button electrode assembly 150 (FIGS. 3C and 3D), clip mount 30 (FIG. 2A), mount 40 (FIG. 2B) or mount 46 (FIG. 2C) comprise hooking element 300. The hooking element is configured to be collapsible while being inserted into tissue, such as tissue of the pancreas, thereby allowing insertion without unnecessarily puncturing the tissue. Once inserted, prongs 304 expand, forming a hook which generally secures the electrode in the tissue. For some applications, use of hooking element 300 replaces the use of sutures and/or glue, as described hereinabove. For other applications, hooking element 300 comprises a suture 306 and a guiding needle 308, which is used to suture the electrode to the tissue with suture 306. After suturing, needle 308 is preferably removed.

FIG. 3G is a schematic illustration of another hooking element 310 of at least one electrode 312, in accordance with a preferred embodiment of the present invention. For some applications, one or more of electrodes 100, such as the electrodes of two-electrode patch assembly 110 (FIG. 3A), single-electrode patch assembly 120 (FIG. 3B), button electrode assembly 150 (FIGS. 3C and 3D), clip mount 30 (FIG. 2A), mount 40 (FIG. 2B) or mount 46 (FIG. 2C) comprise hooking element 310. The hooking element comprises a spiral stopper that generally secures the electrode in the tissue. Hooking element 310 preferably comprises a suture 314 and a guiding needle 316, which is used to suture the electrode to the tissue with suture 314. After suturing, needle 316 is preferably removed. For some applications, a single hooking element secures more than one electrode 312.

FIG. 3H is a schematic illustration of a corkscrew electrode 320, in accordance with a preferred embodiment of the present invention. For some applications, one or more of electrodes 100, such as the electrodes of two-electrode patch assembly 110 (FIG. 3A), single-electrode patch assembly 120 (FIG. 3B), button electrode assembly 150 (FIGS. 3C and 3D), clip mount 30 (FIG. 2A), mount 40 (FIG. 2B) or mount 46 (FIG. 2C) comprise hooking element 310. The corkscrew is screwed into tissue of the pancreas in order to secure the electrode firmly and provide good mechanical gripping. When electrode 320 is used with or as a component of a patch, the electrode is connected by a wire to the patch or directly attached to the electronics of the patch. Preferably, the electrode comprises an insulated wire, of which only a relatively small area is electrically exposed, such as an area 322 of the corkscrew or an area 324 of the wire near the corkscrew. For some applications, the electrode comprises multiple wires separately coated, each wire with a single area electrically exposed, such that the areas are non-overlapping. These areas are used in pairs for differential measurements or individually to obtain multiple single measurements.

FIG. 3I is a schematic illustration of an electrode assembly 330, in accordance with a preferred embodiment of the present invention. Electrode assembly 330 comprises at least two wires 302, which are electrically insulated, preferably coated with 10% Teflon. Wires 302 preferably comprise a biocompatible material, such as platinum/iridium (Pt/Ir), titanium, titanium nitride, or MP35N, and are preferably have a diameter of between about 0.05 and about 0.15 mm, most preferably of about 0.1 mm. A portion of the coating of each wire is removed, exposing an area that serves as an electrode 306. Preferably, the length D₂₁ of each electrode 306 is between about 0.3 and about 0.7 mm, most preferably about 0.5 mm. Pairs of two electrodes 306 preferably are used for taking differential measurements. When the assembly comprises exactly two wires 302, as shown in FIG. 3I, a distance D₂₂ of between about 2 and about 3 mm preferably separates the two electrodes.

The assembly further comprises a suture 304, which preferably comprises braided metal or silk. A needle 308 is attached to the end of the suture, for suturing electrode assembly 330 to tissue of the pancreas. After suturing, needle 308 is preferably removed. The distal ends of wires 302 preferably are joined in a shrink wrapping or connecting element 310 by glue, such as epoxy glue; suture 304 passes through (as shown) or adjacent to connecting element 310. The proximal ends of the wires are electrically and mechanically coupled to a preamplifier or amplifier 312. The proximal end of the suture is preferably mechanically coupled to the amplifier. A cable 314 is connected at one end of the cable to the proximal end of the amplifier. The other end of the cable is connected to an implanted patch or to a control unit. (For wireless transmission applications, the cable may be replaced by data transmission apparatus.) Preferably, the length D₂₃ of the amplifier is between about 3 and about 4 mm. The distance D24 between the amplifier and connecting element 310 is preferably between about 15 and about 25 mm, most preferably about 20 mm. All electrical components of electrode assembly 330, other than electrodes 306, are preferably isolated against fluid, such as by using an epoxy or Parylene.

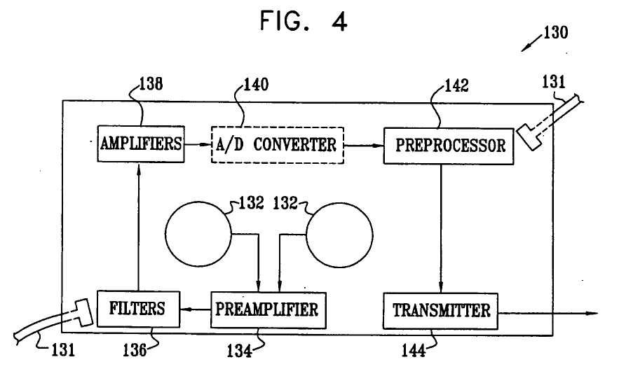

FIG. 4 is a schematic block diagram of a signal-processing patch assembly 130, for implantation on the pancreas, in accordance with a preferred embodiment of the present invention. Preferably, signal-processing patch assembly 130 is attached to tissue of the patient using sutures 131, in a manner similar to that described hereinabove with reference to FIGS. 3A and 3B. Electrode patch 130 comprises one or more electrode assemblies 132, such as two monopolar electrode assemblies 115 (FIG. 3A) or one bipolar concentric electrode assembly 125 (FIG. 3B), or other electrodes known in the art or described herein.

Signal-processing patch assembly 130 additionally comprises signal-processing components, such as a preamplifier 134, filters 136, amplifiers 138, a preprocessor 142, and a transmitter 144, all preferably physically located on the patch assembly. In embodiments in which signal-processing patch assembly 130 comprises two electrode assemblies 132, both electrode assemblies are preferably connected to a single preamplifier 134. Preferably, the electrodes of electrode assemblies 132 are in direct physical contact with the inputs of preamplifier 134, with substantially no wires used for connection. Alternatively, the electrodes of electrode assemblies 132 are connected to the inputs of preamplifier 134 using wires. Signals generated by preamplifier 134 are preferably passed through filters 136 and then amplifiers 138. Filters 136 preferably comprise a high-pass filter, a low-pass filter, and a notch filter (not shown). The high-pass filter preferably has a frequency cutoff of about 0.05 Hz to about 10 Hz, e.g., 0.5 Hz, and the low-pass filter preferably has a frequency cutoff of about 40 Hz to about 500 Hz, e.g., 100 Hz. The notch filter is preferably configured to filter out the frequency of the local power grid, such as 50 or 60 Hz. Amplifiers 138 comprise a single amplifier, or, alternatively, a first-stage and second-stage amplifier (together, a dual-stage amplifier). Preferably the first- and second-stage amplifiers amplify, for example, by about 25× and about 50×, respectively, so as to generate a total amplification of between about 100× and about 10,000×. For some applications, signal-processing patch assembly 130 comprises an analog-to-digital converter 140, in which case preprocessor 142 and transmitter 144 are digital components. Amplifiers 138 send signals to preprocessor 142, either directly, or, if signal-processing patch assembly 130 comprises analog-to-digital converter 140, through the converter. Preprocessor 142 sends signals to transmitter 144.

For some applications, transmitter 144 transmits the generated signals to control unit 90. Alternatively, transmitter 144 transmits the signals directly to an external or implanted treatment unit, as described hereinabove. Transmitter 144 preferably transmits using transmission techniques known in the art, such as inductive transmission, near-field electromagnetic transmission, or radiofrequency transmission.

Alternatively, some or all of the signal-processing components of signal-processing patch assembly 130 are provided on a separate signal-processing patch assembly (not shown) that is connected to the electrodes of two-electrode patch assembly 110 (FIG. 3A), single-electrode patch assembly 120 (FIG. 3B), button electrode assembly 150 (FIGS. 3C and 3D), clip mount 30 (FIG. 2A), mount 40 (FIG. 2B), mount 46 (FIG. 2C), or other device used to attach the electrodes to the pancreas. This signal-processing patch is preferably sutured to a surface near the electrodes, such as another area of the pancreas or the duodenum, for example. Further alternatively, the electrodes comprise an array of implanted electrodes, and circuitry on a patch or in control unit 90 combines data generated by the array. In this case, each electrode or pair of electrodes is connected to a dedicated preamplifier, or multiple electrodes or pairs of electrodes share a preamplifier, such as by using time-multiplexed input to the preamplifier. In embodiments comprising button electrode assemblies 150, preamplifier 160 (FIG. 3C) is preferably located on patch 156 or on the separate signal-processing patch assembly.

In a preferred embodiment of the present invention, apparatus 18 undergoes a calibration procedure. In a typical initial calibration procedure, a bolus dose of glucose is administered to the patient, and electrical function analysis block 82 determines changes in the electrical activity of the pancreas responsive to the glucose. (Experimental results showing some such changes in activity are described hereinbelow.) Parameter search and tuning block 84 subsequently modifies a characteristic (e.g., timing, frequency, duration, magnitude, energy, and/or shape) of the signals applied through one of electrodes 100, typically so as to cause the pancreas to release a hormone such as insulin in greater quantities than would otherwise be produced. This release causes cells throughout the patient's body to increase their uptake of the glucose, which, in turn, lowers the levels of glucose in the blood and causes the electrical activity of the pancreas to return to baseline values. In a series of similar calibration steps, block 84 repeatedly modifies characteristics of the signals applied through each of the electrodes, such that those modifications that reduce blood sugar, accelerate the return of the electropancreatographic measurements to baseline values, and/or otherwise improve the EPG signals, are generally maintained, while modifications that cause it to worsen are typically eliminated or avoided.

It will be appreciated that whereas the calibration procedure described hereinabove is applied with respect to a single electrode, for some applications, multiple electrodes are calibrated substantially simultaneously, for example, in order to determine which electrodes should be driven simultaneously to apply current to the pancreas.

Optionally, during the initial calibration procedure, the locations of one or more of electrodes 100 are varied while EPG signals are measured and/or electrical signals are applied therethrough, so as to determine optimum placement of the electrodes.

Alternatively or additionally, the calibration procedure includes: (a) administration of insulin and/or a fasting period to reduce blood sugar levels, (b) detection of changes in pancreatic electrical activity responsive to the reduced blood sugar levels, and (c) application of electrical signals to the pancreas configured to enhance glucagon production and generally restore the EPG signals to their baseline values.

Preferably, the calibration procedure is additionally performed by a physician or other healthcare worker at subsequent follow-up visits and by unit 90 automatically during regular use of the apparatus (e.g., once per day, before and/or after a meal, or before and/or after physical activity), mutatis mutandis. When apparatus 18 is calibrated in the presence of a physician or healthcare worker, it is often desirable to administer to the patient glucose boluses having a range of concentrations, in order to derive a broader range of operating parameters, which are stored in control unit 90 and can be accessed responsive to signals from the sensors and electrodes coupled to the control unit.

It is to be understood that where preferred embodiments of the present invention are described herein with respect to glucose and insulin, this is by way of example only. In other embodiments, the effects of other chemicals, such as glucagon or somatostatin, on pancreatic electrical activity are monitored, and/or signals are applied to the pancreas so as to modulate the release of other hormones, such as glucagon or somatostatin. Additionally, for some applications, during calibration, glucose, insulin, a diazoxide-like compound, tolbutamide, and/or other chemicals that affect blood levels of glucose and/or insulin, are administered orally or intravenously.

Preferably, during calibration and during regular operation of control unit 90, a systemic function analysis block 80 of control unit 90 receives inputs from supplemental sensors 72, and evaluates these inputs, preferably to detect an indication that blood sugar levels may be too high or too low. Alternatively or additionally, block 80 evaluates these inputs to detect indications that insulin, glucagon, and/or somatostatin may be too high or too low. If appropriate, these inputs may be supplemented by user inputs entered by the patient through operator controls 71, indicating, for example, that the patient senses that her blood sugar is too low. In a preferred embodiment, parameter search and tuning block 84 utilizes the outputs of analysis blocks 80 and 82 in order to determine parameters of the signals which are applied through electrodes 100 to pancreas 20.

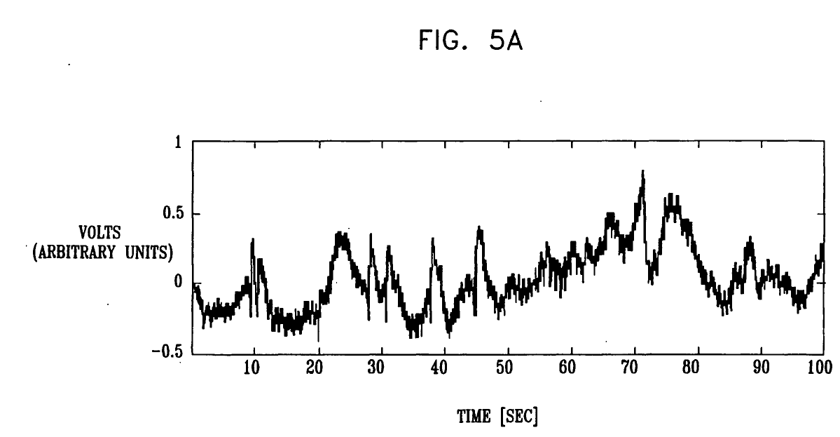

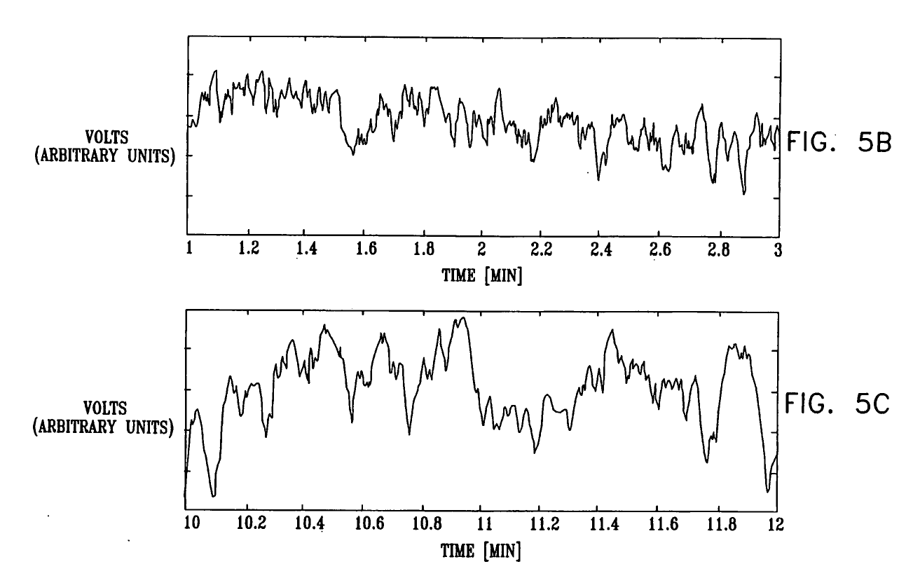

FIGS. 5A, 6A, 7B, and 7C are graphs showing in vivo experimental results measured in accordance with a preferred embodiment of the present invention. A sand rat (psammomys) was anesthetized with 40 mg/ml (0.15 mg/100 mg body weight) pentobarbital. The right jugular vein was cannulated to allow drug or glucose injections, and to allow blood samples to be taken for glucose concentration measurements. The animal was positioned on a warmed (37° C.) table. A laparotomy was performed, and the pancreas was displaced from the abdomen and put in a dish on top of an electrode set similar to that shown in FIG. 2C, while retaining anatomical connection to the rest of the body of the sand rat. By removing the pancreas from the body, breathing and ECG artifacts were reduced. Surface electrodes like those shown in FIG. 2A were carefully attached to the pancreas, and an additional set of electrodes like those shown in FIG. 2B were placed above the pancreas. The surgery and electrode placement were performed using surgical binoculars. In order to minimize electrical and mechanical noise, the sand rat was put inside a Faraday cage, and electrical measurements were performed on a pneumatic table.

The electrodes were connected to a Cyber-Amp 320 (Axon Instruments) amplifier, in which total gain was set to 10000 and a band pass filter was to allow 0.1 to 40 Hz signals to pass. The Cyber-Amp was connected to a computer, and recorded signals which were sampled at 1000 Hz and saved for off-line analysis.

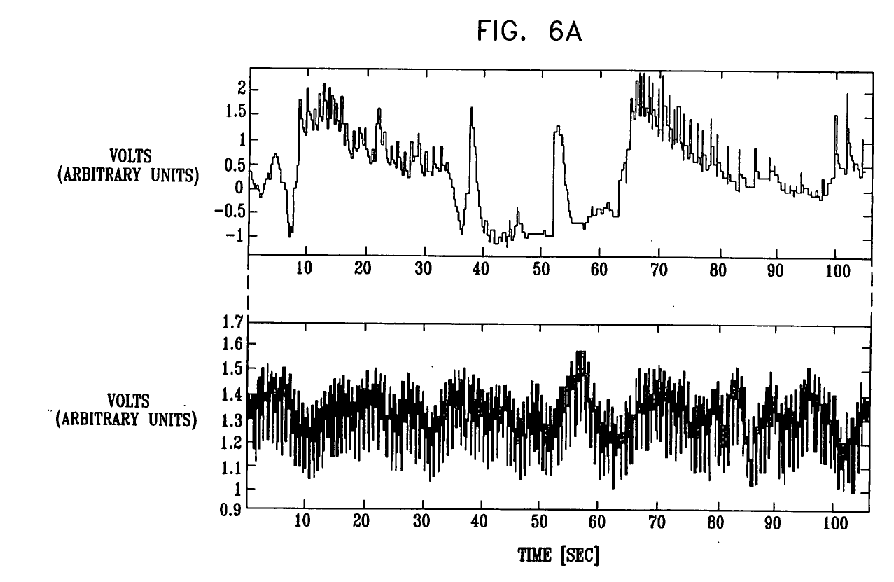

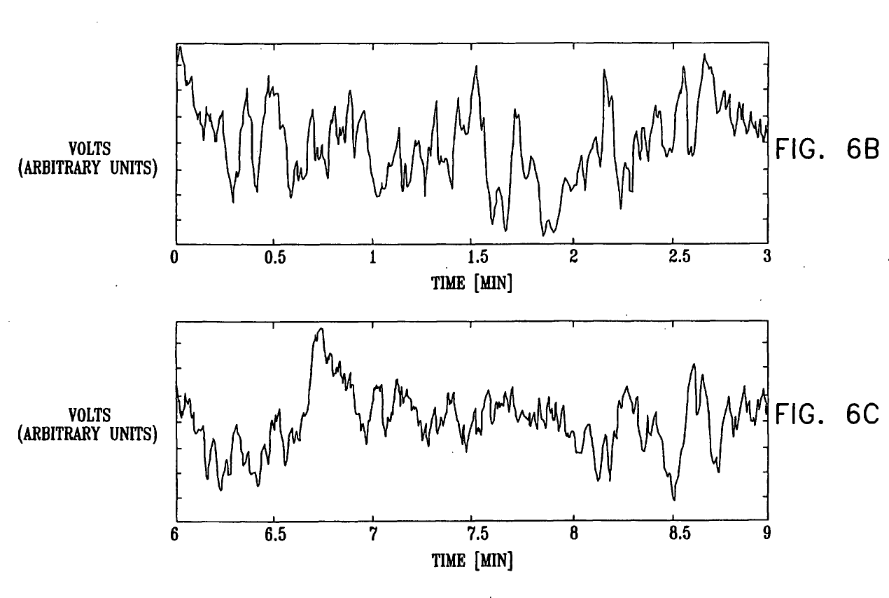

FIGS. 5A and 6A show bipolar pancreatic readings made at different times during experiments performed without the administration of glucose or any drug. It is noted that spikes of different widths (i.e., durations) are present in FIG. 5A, most being substantially longer, infrequent, and generally irregular than most of the spikes seen in FIG. 6A (e.g., those spikes generated at times t between 65 and 80 seconds). Much of the activity seen in FIG. 6A is characterized by sharply-rising spikes having durations between about 200 and about 500 milliseconds, which are produced at a variable spike-generation rate having a mean value of about 1 Hz. The absolute amplitudes of the spikes are generally several tens of microvolts. As described in greater detail hereinbelow, waveform characteristics (such as spike widths) are preferably interpreted by a control unit to yield information about the activity of the various types of cells in the pancreas. For example, as shown in figures in the above-cited article by Nadal, beta cells typically produce spikes having widths which are markedly smaller than those of alpha cells. Alternatively or additionally, duration aspects and/or magnitude aspects of other features of the recorded waveform are analyzed to facilitate a determination by the control unit of the contribution of different types of pancreatic cells to the measured EPG signals.

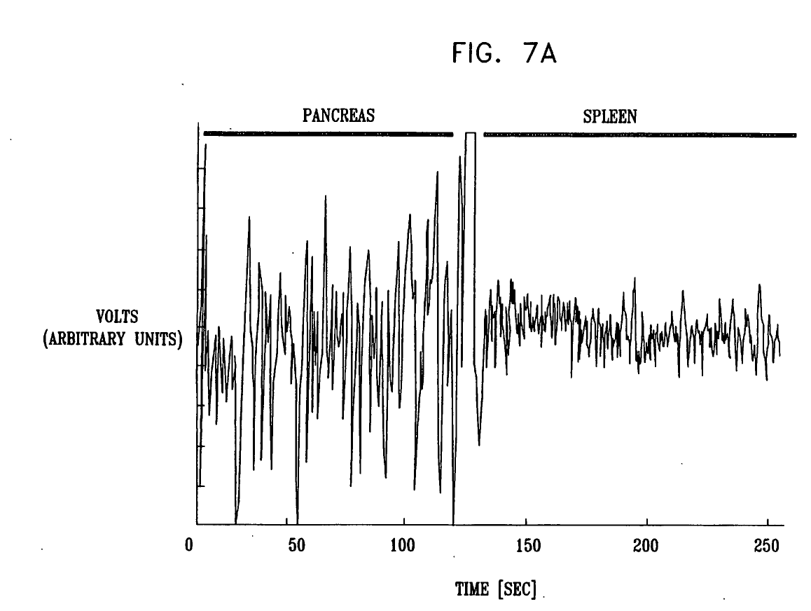

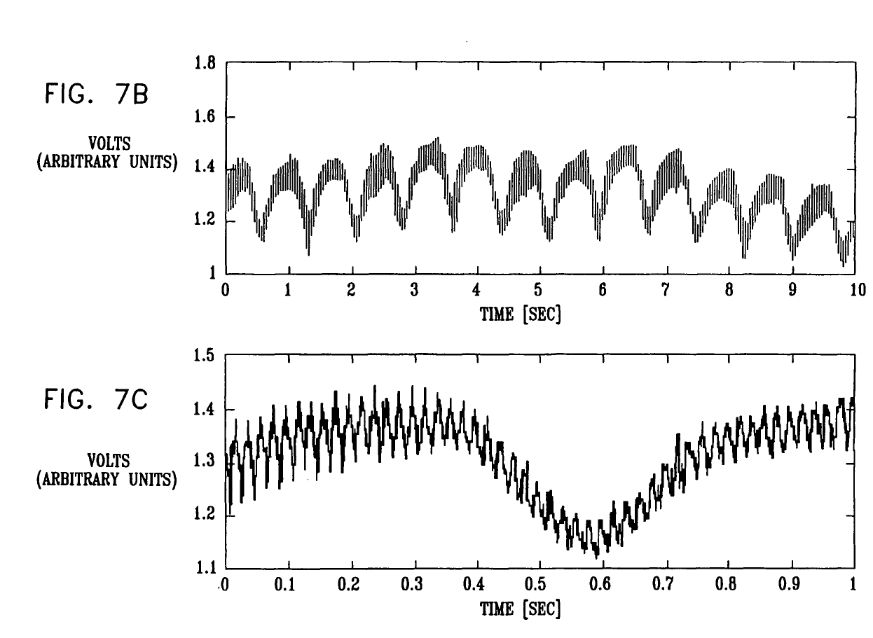

The lower trace in FIG. 6A shows noise measured by electrodes at a different site on the pancreas. To increase clarity, the time axis of this trace is expanded in FIG. 7B, and even further in FIG. 7C. The predominant features in FIG. 7B arise from breathing of the animal, while those in FIG. 7C are a result of power-line noise. It is noted that each of these is significantly different from the various pancreatic readings shown in the figures of the present patent application, and that software running in the control unit is preferably configured to identify and filter out any such non-pancreatic electrical activity.

FIGS. 5B, 5C, 6B, 6C, 7A, 8A, 8B, and 8C are graphs illustrating experimental data obtained in accordance with a preferred embodiment of the present invention. In these experiments, a rat was anesthetized, an abdominal incision was made in the animal, and the pancreas was removed from the rat's abdomen and placed in a Petri dish adjacent to the rat. Care was taken to assure that the major blood vessels connected to the pancreas were not cut or significantly disturbed during this procedure. The pancreas was removed so as to minimize the interference of the motion of breathing or other movements on the measurements being made. While in the Petri dish, the pancreas was continuously bathed in a warm saline solution.

Bipolar titanium wire electrodes, 300 microns in diameter, were placed in a mount similar to that shown in FIG. 2A. The mount was placed on the head of the pancreas, in such a manner that the electrodes were sensitive to, it is believed, the electrical activity of at least several islets of Langerhans. In order to reduce electrical noise artifact, a sensing electrode was placed on the animal's spleen (in situ), which is substantially not electrically active. The data shown in FIGS. 5B, 5C, 6B, and 6C are voltage measurements reflecting the difference between the voltages measured on the pancreas and on the spleen.

The data in FIG. 5B represent a 2 minute baseline data collection period, in which the bipolar electrodes described hereinabove were held against the pancreas while data were recorded. Subsequently, a 20% glucose solution was injected into the rat. Pancreatic electrical activity subsequent to the injection is shown in FIG. 5C. A number of changes are seen between the baseline data and the post-injection data, including changes in frequency components of the recorded signal, as well as changes in magnitudes of fluctuations of the signal.

The data in FIG. 6B represent a 3 minute baseline data collection period, in which the bipolar electrodes were held against the pancreas while data were recorded. Subsequently, a 20% glucose solution was used to bathe the pancreas (rather than being injected into the rat). Pancreatic electrical activity subsequent to this administration of glucose is shown in FIG. 6C. A number of changes are seen between the baseline data and the post-glucose-administration data, including changes in frequency components of the recorded signal, and changes in magnitudes of fluctuations of the signal. In a preferred embodiment of the present invention, control unit 90 is adapted to analyze recorded electropancreatographic data so as to determine changes in the frequency components of the signal, and changes in magnitudes of fluctuations of the signal, which are indicative of changes in a patient's blood sugar.

It is hypothesized that increases in amplitudes and/or fluctuations of the recorded signals may correspond to “recruitment” (activation) of increasing numbers of cells in increasing numbers of islets of Langerhans, which in turn corresponds to the propagation of glucose through the pancreas.

FIG. 7A shows the sensitivity of the measurement apparatus used in these rat experiments to the electrical activity of the pancreas and the spleen. The data shown in FIG. 7A represent electrical readings from the pancreas from t=0 to approximately t=120 seconds. Following this initial period, the electrodes were removed from the pancreas and placed on the spleen, and splenic electrical activity was recorded from t=about 140 to about 250 seconds. The pancreas is seen to be significantly more electrically active than the spleen. In continuations of this experiment (not shown), each time the electrodes were moved from the pancreas to the spleen, the electrical activity was seen to decrease. Additionally, when the electrodes were moved back to the pancreas, activity increased. This graph indicates that the electrical activity measured by electrodes on the pancreas do, in fact, measure pancreatic electrical activity, and are not simply recording electric currents whose source is outside the pancreas. If the latter were the case, then similar activity would be expected to be seen on the spleen.

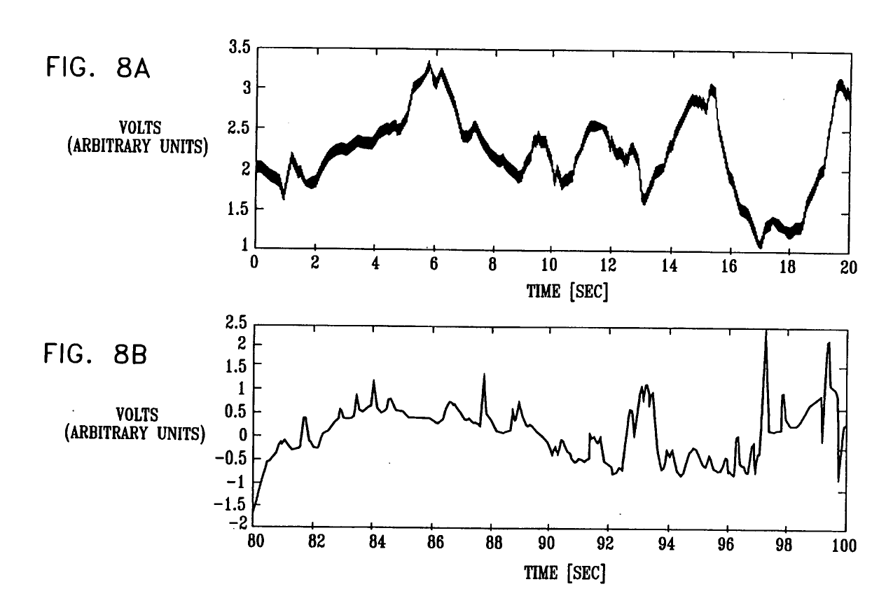

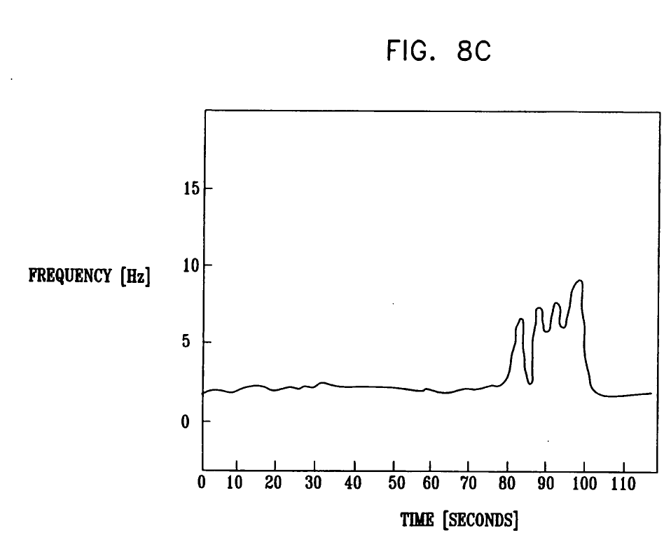

FIG. 8A shows electrical activity recorded in a sand rat during a first period (0-20 seconds). At approximately t=20 seconds, tolbutamide was injected. FIG. 8B shows pancreatic electrical activity during a second period (80-100 seconds), following this injection. It is noted that some frequency components are readily observable in FIG. 8B which are not present in FIG. 8A. FIG. 8C shows the results of a frequency analysis of all of the data, from 0 to 120 seconds. Dominant frequency components are clearly seen to change during the period following the injection of tolbutamide. In a preferred embodiment of the present invention, control unit 90 is adapted to analyze recorded electropancreatographic data so as to determine changes in the frequency components of the signal which are indicative of changes in a patient's blood sugar.

In the experiment whose results are shown in FIGS. 8A, 8B, and 8C, the effect of tolbutamide to increase pancreatic electrical activity, so as to stimulate insulin production and/or secretion, simulates the effect of high blood sugar to stimulate insulin production.

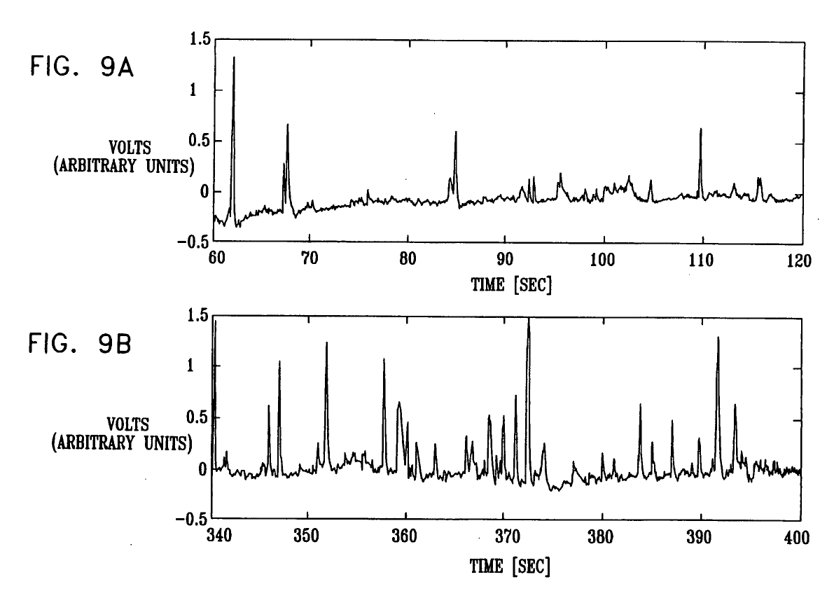

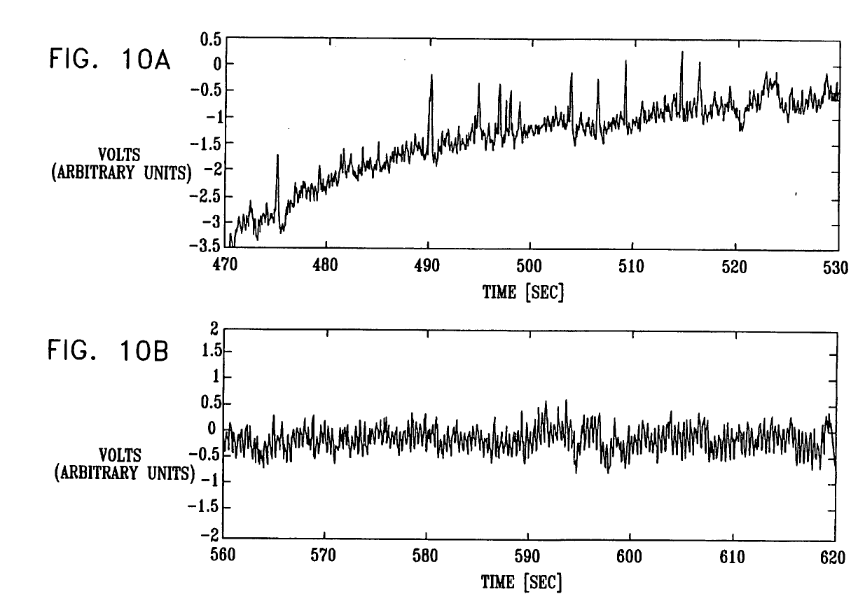

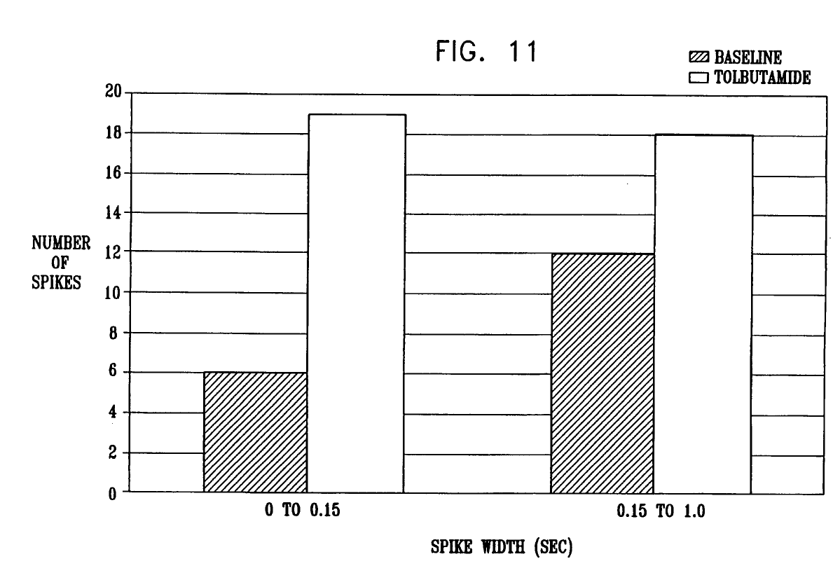

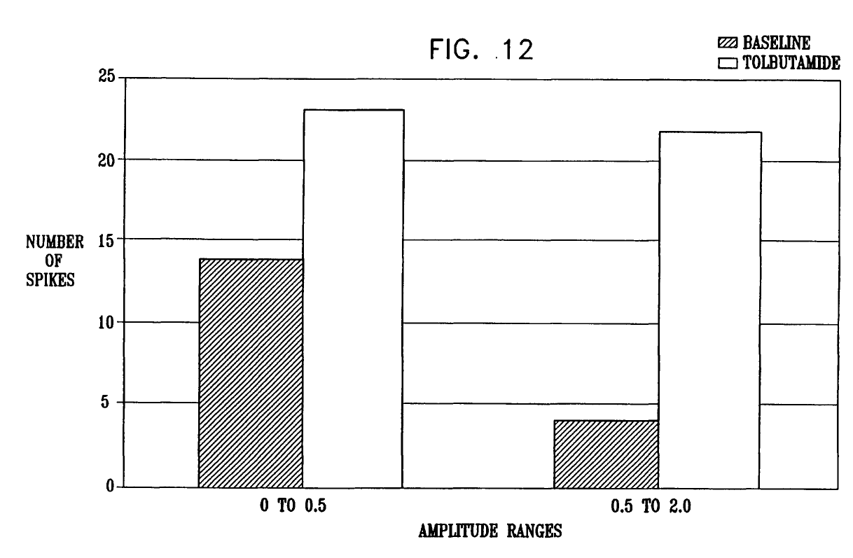

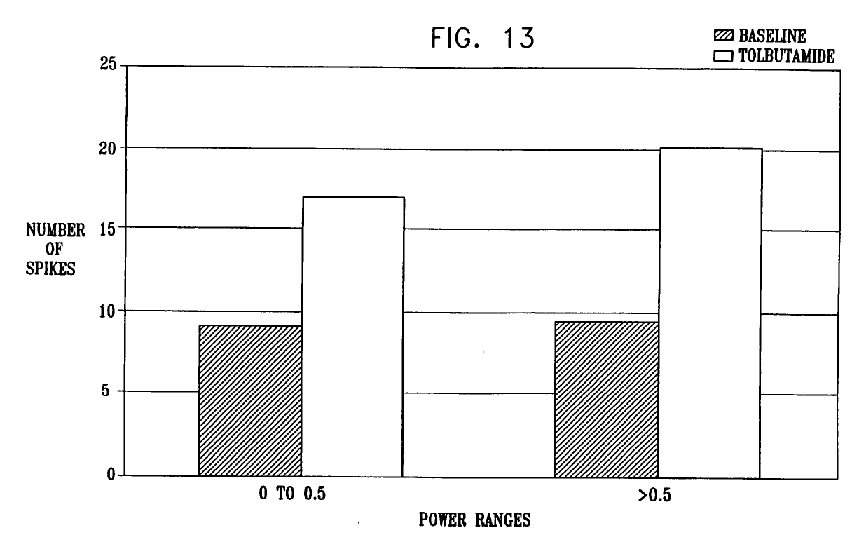

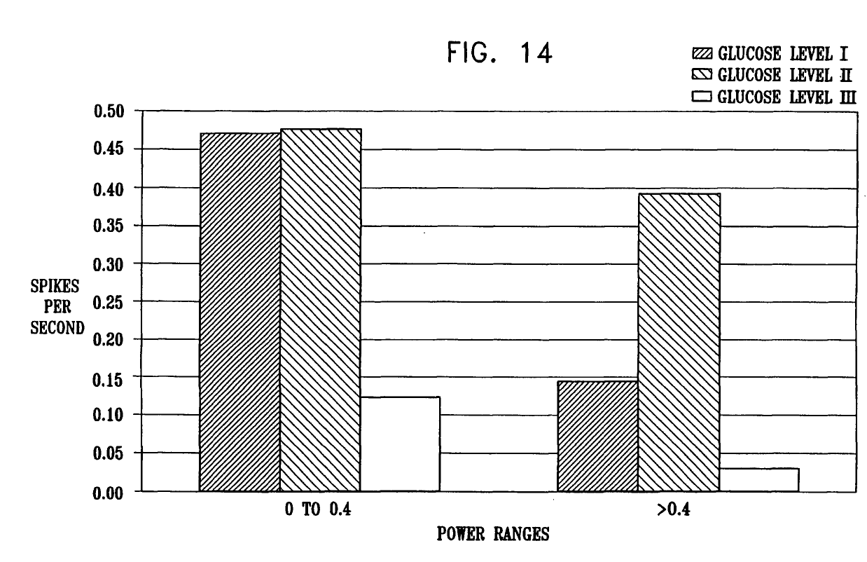

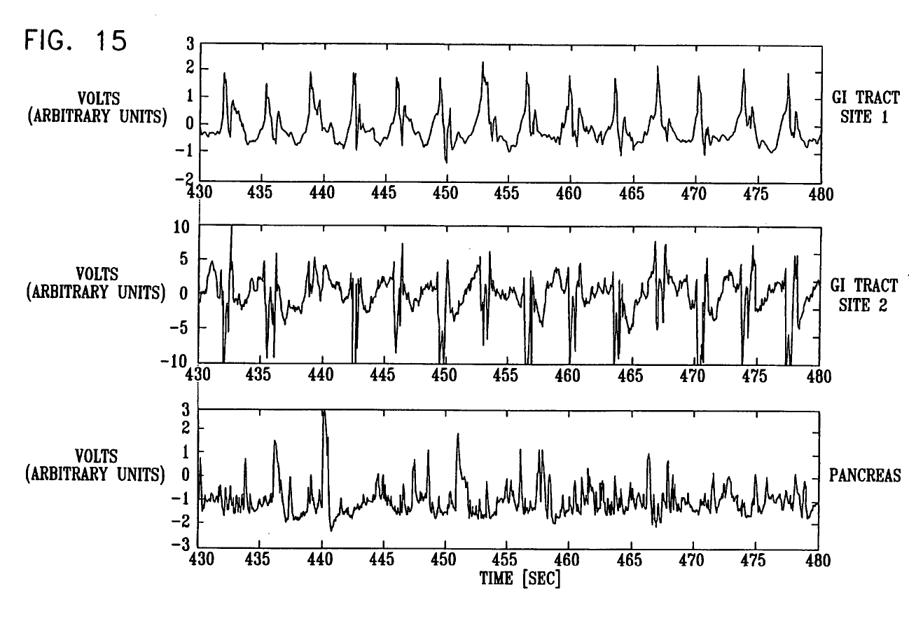

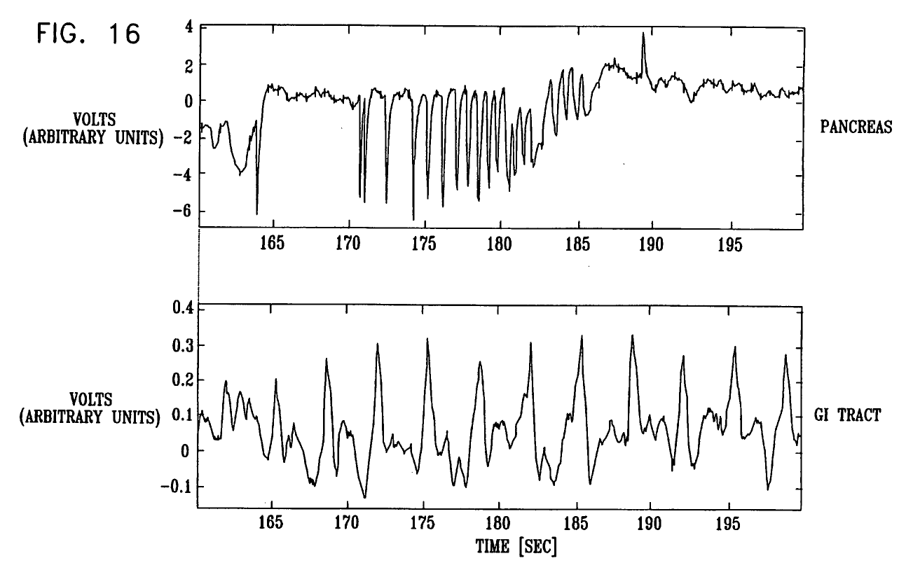

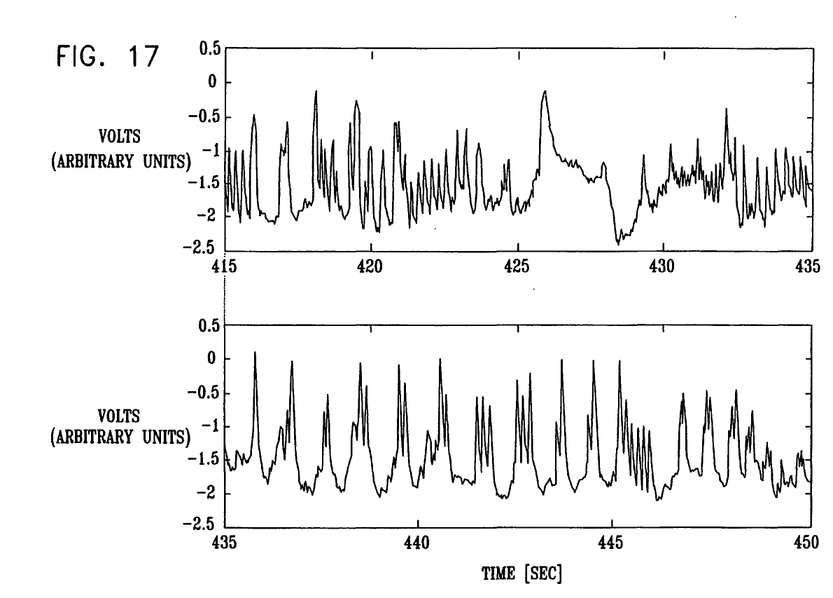

FIGS. 9A, 9B, 10A and 10B are graphs illustrating additional experimental data obtained in accordance with a preferred embodiment of the present invention. The experiments were performed upon sand rats under laboratory conditions similar to those of the experiments described above with reference to FIGS. 5B, 5C, 6B, 6C, 7A, 8A, 8B, and 8C. FIG. 9A shows a 2 minute baseline electrical activity data collection period, in which the bipolar electrodes on the pancreas recorded electrical activity. At approximately t=100 seconds, the sand rat was injected with a dose of tolbutamide (0.1 cc, 5 mM) through the jugular vein, in order to stimulate pancreatic electrical activity and thereby to increase the release of insulin. FIG. 9B shows data recorded through the same electrodes, beginning at four minutes after the tolbutamide injection. In FIG. 9B, a clear increase of electrical activity is observed in response to the administration of tolbutamide. In particular, spike generation is seen to substantially increase.

FIG. 10A shows a one minute baseline date collection period, in which the electrical activity of the pancreas of a sand rat was measured under similar laboratory conditions. At t=530 seconds, the sand rat was injected with diazoxide (0.1 cc), in order to reduce pancreatic electrical activity and thereby reduce the production and/or secretion of insulin. FIG. 10B, which shows data starting from thirty seconds following this injection, shows a marked decrease in pancreatic electrical activity. In particular, spike generation is seen to be essentially terminated. The combined results of FIGS. 9A, 9B, 10A, and 10B show that electropancreatography, as provided by these embodiments of the present invention, can be used to allow a control unit implanted in a patient's body to determine in real-time whether the pancreas is behaving in a manner indicative of elevated blood sugar or depressed blood sugar. In a preferred embodiment of the present invention, control unit 90 is adapted to analyze recorded electropancreatographic data so as to determine changes in a frequency of spike generation, which are indicative of changes in the production and/or secretion of insulin by the pancreas of a patient. Preferably, responsive to such a determination, control unit 90 (a) directly stimulates the pancreas so as to modulate insulin, somatostatin or glucagon production, (b) initiates other measures for restoring the pancreatic homeostasis, e.g., directs the patient to inject insulin or call for professional help, (c) stores recorded data to allow subsequent analysis, and/or (d) applies another treatment, such as those described hereinabove.

FIGS. 11, 12, and 13 show the results of signal processing of the experimental results shown in FIGS. 9A and 9B, in accordance with a preferred embodiment of the present invention. The width (duration) of each of the spikes measured during the experiment (of which the data shown in FIGS. 9A and 9B are a subset) was used as an indicator for dividing the spikes into two groups: Group I, those spikes having widths less than 0.15 second, and Group II, those spikes having widths ranging from 0.15 to 1.0 second. It can be seen in FIG. 11 that, for all ranges of measured spike width, the number of spikes after injection of tolbutamide is notably greater than prior to the tolbutamide injection. In a preferred embodiment of the present invention, control unit 90 detects a systemic physiological change in a patient (e.g., changes in blood sugar or blood insulin level) by detecting an increase in generation of spikes within a given range of widths.

A similar analysis was performed with respect to the amplitudes of the spikes before and after tolbutamide injection. FIG. 12 shows that tolbutamide injection induces more large amplitude and small amplitude spikes than are present in the baseline state. In a preferred embodiment of the present invention, control unit 90 detects a systemic physiological change in a patient (e.g., changes in blood sugar or blood insulin level) by detecting a change in a ratio of large amplitude to small amplitude spikes.

FIG. 13 is based on further analysis analogous to that shown in FIGS. 11 and 12. The width (i.e., duration) and the amplitude of each spike in FIGS. 9A and 9B were multiplied, so as to generate a measure of the power of the spike. It is seen that the injection of tolbutamide yields approximately twice the number of spikes relative to baseline, in the measured power ranges. These results indicate that electropancreatography, as provided by embodiments of the present invention, generates a quantitative indication of a condition of the blood. In a preferred embodiment of the present invention, this form of analysis is used by control unit 90 to determine the onset and extent of glucose changes in the blood, mutatis mutandis.

FIG. 14 provides further support for this conclusion. In vivo, in situ, experiments were performed on the pancreas of a dog, in accordance with a preferred embodiment of the present invention. In these experiments, a portion of the outer layer of connective tissue surrounding the pancreas was removed, and surface electrodes were placed directly on the dog's pancreas. Results are shown in FIG. 14. In these experiments, three different levels of blood glucose were measured: Level I was approximately 170 mg/dL, Level II was approximately 220 mg/dL, and Level III was approximately 500 mg/dL. Electrical activity of the pancreas was measured responsive to each of the glucose levels. FIG. 14 shows the results of signal processing of the measured electrical activity similar to that described with reference to FIG. 13. It can be seen in FIG. 14 that the different glucose levels result in measurable differences in pancreatic electrical reaction, as indicated by spikes per second. In particular, the excessively-high Level III protocol appears to either suppress spike generation, or not to facilitate it to the same extent as Levels I and II. In addition, glucose concentrations at Level II are seen to induce “high-power” spikes at over twice the rate of either Level I or Level III. Thus, FIG. 14 demonstrates that electropancreatography can be used to monitor the level of glucose in the blood. In clinical use, electropancreatographic readings would preferably be taken over a range of imposed glucose levels during calibration, so as to enable subsequent accurate assessments by the control unit of the patient's glucose levels. In a preferred embodiment of the present invention, control unit 90 detects a changes in blood sugar by detecting a change in a frequency of the occurrence of spikes (spikes per second).

FIG. 15 shows results of a further experiment carried out in accordance with a preferred embodiment of the present invention. In order to ensure that the results of the above experiments and clinical electropancreatographic measurements do not include excessive electrical artifact due to electrical activity of smooth muscle in the vicinity of the pancreas, such as that of the gastrointestinal (GI) tract, measurements were made of the electrical activity at two sites in the GI tract simultaneous with the electropancreatographic measurements. The top and middle traces of FIG. 15 show the electrical activity at two sites on the GI tract of a dog, and the bottom trace shows the electrical activity of the pancreas, measured simultaneously with the GI tract measurements. It is markedly clear that the electrical activity of the GI tract is strongly periodic in nature, each GI site having the same period, while the pancreatic activity is independent of the GI tract. In the dog experiments described herein, a clip including a small metal spring was used to hold the electrode mounts to the pancreas.

FIG. 16 shows results of yet a further experiment on a dog, comparing electropancreatographic readings with electrical activity measured at a site on the GI tract, in accordance with a preferred embodiment of the present invention. The electrical activity of the GI tract is distinctly periodic while the pancreas exhibits characteristic frequency changes. In particular, it is noted that the EPG trace shows a period of minimal pancreatic activity from t=165-170 seconds, which is followed by an approximately ten-second period in which spikes occur at continually increasing frequencies. This characteristic of the pancreas is both different from typical GI tract behavior, and has been seen by the inventors to recur in numerous experiments performed in accordance with preferred embodiments of the present invention. In clinical use, in a preferred embodiment of the present invention, control unit 90 monitors changes in the spike frequency responsive to a series of imposed or other conditions (such as particular glucose levels or changes in glucose levels), in order to determine those characteristic changes in spike frequency which are indicative that a treatment should be initiated or a warning signal should be generated. For example, in the calibration period for a given patient or during regular use, any one or more of the following may be found to be useful indicators of blood glucose level or changes thereof:
a rate of spike generation,
aspects of the widths (i.e., durations) of one or more spikes,
aspects of morphology of a measured waveform,
changes (e.g., increases or decreases) in the rate of spike generation,
particular spike magnitudes associated with particular spike frequencies or with changes in spike frequencies,
changes in spike magnitudes associated with particular spike frequencies or with changes in spike frequencies,
changes in the magnitudes of one or more frequency components, even in the absence of spikes, or
frequency or changes in frequency of spikes having particular spike widths, e.g., those widths which are predominantly characteristic of alpha-, beta-, delta-, or polypeptide-cell activity.

The GI tract data shown in FIGS. 15 and 16 are generally consistent with measurements of electrical activity of smooth muscles surrounding blood vessels made by several researchers and published in articles, such as those cited in the Background section of the present patent application by Lamb, F. S. et al., Zelcer, E., et al., Schobel, H. P., et al., and Johansson, B. et al.

FIG. 17 shows pancreatic electrical activity of a dog, measured in accordance with a preferred embodiment of the present invention. This data set is further indication that it is feasible to measure the electrical activity of a substantial portion of the pancreas and that the pattern of such activity is markedly different from the characteristic approximately 0.3 Hz electrical activity of the smooth muscle of the GI tract. In a preferred embodiment of the present invention, the effects of artifact due to various physiological factors such as smooth muscle electrical activity, neural activity, cardiac muscle activity and respiration, which are inherently distinguishable from pancreatic electrical activity because of their different characteristics, are reduced by (a) the use of reference electrodes placed on or near a source of electrical artifact, or (b) software in the control unit which is operative to detect non-pancreatic waveforms and remove them from the EPG signals.

In a preferred mode of analysis, control unit 90 analyzes the EPG signals so as to distinguish between portions thereof which are indicative of activity of alpha cells and beta cells of the pancreas. For some applications, analysis is also performed to determine changes in delta cell activity and/or polypeptide cell activity. Increases in beta cell activity typically are interpreted by the control unit to be indicative of the generation of insulin responsive to increased blood sugar, while increases in alpha cell activity typically correspond to the generation of glucagon responsive to decreased blood sugar. If appropriate, a treatment may be initiated or modified based on these determinations.

Figures in the above-cited article by Nadal show calcium-based fluorescence changes responsive to alpha, beta, and delta cell activity. Each cell produces its own characteristic form, which distinguishes it from the other types of cells. A particular distinguishing characteristic is the duration of each burst of electrical activity. In the Nadal article, alpha cells are seen to produce substantially more prolonged, long-duration bursts of fluorescence than do beta cells, whose activity is better characterized as a series of short-duration spikes. The data presented in the figures of the present patent application can also be analyzed to distinguish between the activity of the different types of pancreatic cells. FIG. 17 shows prolonged, long-duration bursts of electrical activity, for example, at 417 seconds and between 425 and 428 seconds, and repeated bursts of short-duration spikes from 435 to 450 seconds. In a clinical setting, such an analysis is preferably performed following a suitable calibration of the EPG apparatus with each patient. The calibration preferably includes administering insulin or glucose in different doses to a patient to produce a range of blood sugar levels, and analyzing the EPG signals to determine characteristics of the spike associated with each blood sugar level.

For some applications, EPG analysis is performed using the assumption that the various inputs to the EPG (e.g., alpha-, beta-, delta-, and polypeptide-cells) are generally mutually-independent. In this case, signal processing methods known in the art, such as single value decomposition (SVD) or principal component analysis, are preferably adapted for use with the techniques describes herein in order to separate the overall recorded activity into its various sources.

Alternatively, for some applications it is preferred to assume that the various components of the EPG are mutually-dependent, in which case techniques such as that described in the above-cited article by Gut are preferably adapted to enable a determination of the contribution to the EPG of alpha cells, beta cells, and/or other factors. In particular, the Gut article describes methods for distinguishing the contributions of individual finite-duration waveforms to an overall electromyographic (EMG) signal. In a preferred embodiment of the present invention, this method is adapted to facilitate a calculation of the contributions of groups of alpha and beta cells to the overall EPG signal.

In a preferred embodiment of the present invention, in combination with or separately from the analysis methods described hereinabove, EPG signals are interpreted by evaluating waveform frequencies, amplitudes, numbers of threshold-crossings, energy, correlations with predefined patterns or with an average pattern, and/or other characteristics.

It will be appreciated that the principles of the present invention can be embodied using a variety of types and configurations of hardware. For example, for some applications, it is appropriate to use a relatively small number of electrodes placed on or in the head and/or body and/or tail of the pancreas. Alternatively or additionally, a larger number of electrodes, e.g., more than ten, are placed on the pancreas, preferably but not necessarily incorporated into flexible or stiff electrode arrays. In a preferred embodiment, several arrays each comprising about 30-about 60 electrodes are placed on or implanted in the pancreas.

It is noted that the pin electrodes used in gathering the data shown in the figures had characteristic diameters of approximately 500 to 1000 microns, which, despite their large size, were able to record electrical activity over relatively long periods, e.g., up to several hours. Any injury which may have been induced (none was detected) would presumably have been limited to a local region around each electrode. For some clinical applications, it is preferable to use or adapt for use commercially-available electrodes such as those which have diameters of several microns and are designed for recording electrical activity in the brain. A range of electrodes are known or could be adapted to measure the characteristic 1-100 microvolt pancreatic electrical activity.

FIG. 18 is a schematic illustration of electrode apparatus used in experiments conducted to sense electrical activity of a pancreas and described hereinbelow with reference to FIGS. 19-40, in accordance with a preferred embodiment of the present invention. Signals were recorded from rats and sand rats in an in situ procedure, in which the test animal was not alive, but in which a physiological solution was perfused into the portion of the aorta which enters the pancreas, and samples were collected from the portal vein in the output of the pancreas. The pancreas was continuously perfused throughout the experiment with a solution that contains glucose, and, if appropriate, other pharmacological agents. (References to “in situ” preparations hereinbelow refer to this experimental protocol.)

It is believed that the data shown in the following figures are not fundamentally dependent on the particular configurations of electrodes which are used. For example, for some experiments (not shown), a suction pipette electrode containing an Ag/AgCl wire was used to measure pancreatic electrical activity with respect to an Ag/AgCl wire reference electrode that was placed under the pancreas.

As shown in FIG. 18, a patch assembly 200 comprises a patch 202, preferably made of silicone, polyamide, or another flexible biocompatible material, and an electrode assembly 204, for use for recording pancreatic electrical activity. The electrode assembly comprises electrode 206, preferably comprising platinum-iridium or titanium, surrounded by an insulating ring 208, such as a glass, silicone or polyamide ring, the outer diameter D₁₈ of which is preferably about 700 microns. Electrode 206 is preferably recessed by a distance D₁₉ of 100-200 microns. Wire electrode 206 is exposed on one side of patch assembly 200, and sensing leads 210 coupled to electrode 206 exit electrode assembly 204 towards the other side of the patch assembly. Preferably, the electrode protrudes from the patch assembly by a distance D₂₀ of between about 100 and about 200 microns. For some of the experiments described with reference to FIGS. 19-40, data were taken with respect to an Ag/AgCl wire reference electrode placed under the pancreas. The electrode may be attached to the pancreas by suction applied through an optional vacuum tube 212 coupled to an optional suction lumen 214 of electrode assembly 204, by being held with an adhesive, with a suture, or simply by being placed on the pancreas. Data shown in FIGS. 19-40 were acquired when patch assembly 200 was applied to the pancreas with suction. Insulating materials placed around the electrode included glass, silicone, and polyamide. Preferably, a cavity 216, disposed around electrode assembly 204, allows any excess adhesive which may have been applied to the silicone patch to collect around the insulating material, without contaminating the electrode itself.

For some pig experiments (not shown), differential recording was performed using two sets of the electrode apparatus shown in FIG. 18, or other electrodes, which were placed approximately 1 mm-1 cm apart on the pancreas. It is believed that inter-electrode spacings of up to approximately 5 cm still provides significant benefit. The use of closely-spaced differential electrodes typically provides a reduction in sources of noise, e.g., cardiac, gastrointestinal or breathing-related noise.

FIGS. 19-40 show graphs of experimental data recorded in accordance with various preferred embodiments of the present invention described hereinbelow. The upper trace of FIG. 19 depicts eight minutes of electrical activity recorded from an in situ rat pancreas exposed to 10 mM glucose, and the lower trace is an expanded view lasting 1.5 seconds, showing details from a single burst seen in the upper trace. It is noted that the frequency of the burst seen in the lower trace is not regular; rather, it is initially high for several spikes, and steadily decreases. In general, the activity in the upper trace can be described as groups of bursts lasting 100 ms to several seconds, separated by silent periods having durations on the order of half a minute. Other experiments have shown silent periods on the order of up to several minutes.

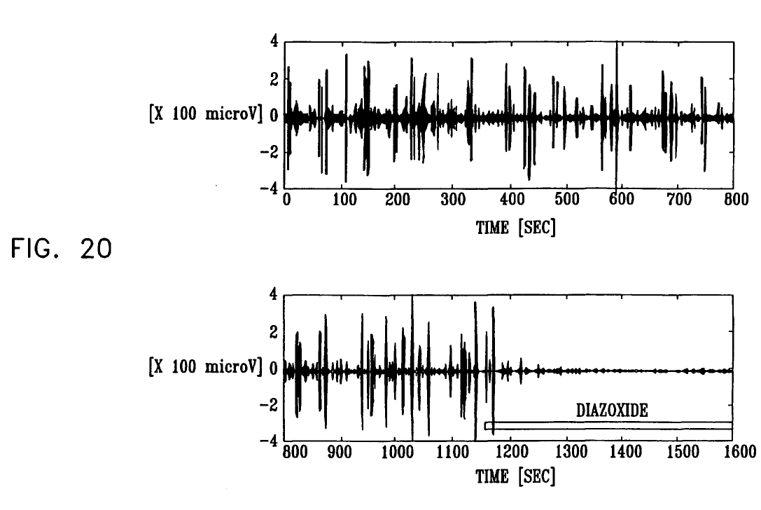

FIG. 20 shows results demonstrating that the recorded electrical activity is of endocrine origin. The figure depicts the activity before and after the administration of Diazoxide (100 uM, with 10 mM glucose) to a rat. Diazoxide is known to open KATP channels, and is seen to cause a significant decrease in the measured electrical activity.

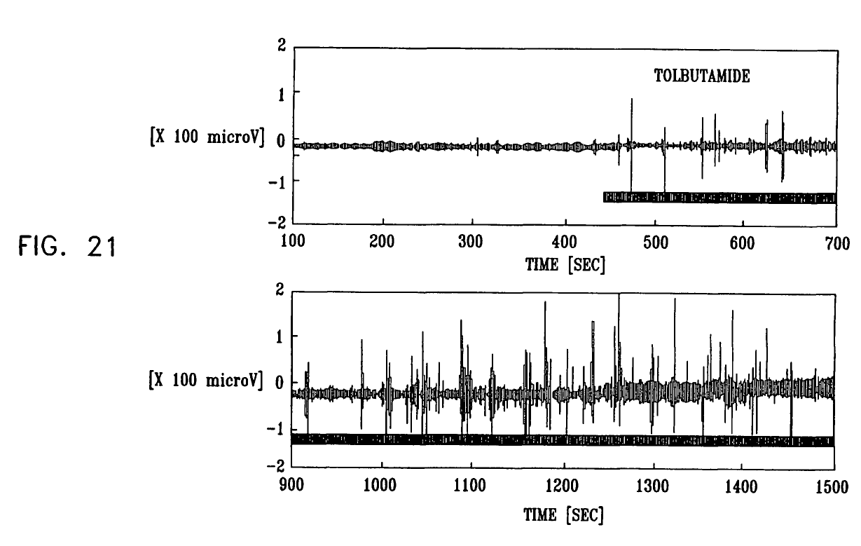

FIG. 21 shows the corresponding, inverse, response to tolbutamide (100 uM, with 10 mM glucose) administered shortly after termination of the administration of Diazoxide to the same rat. Tolbutamide is known to close KATP channels. This in turn causes depolarization, and the increase in pancreatic electrical activity seen in the figure. It is clearly seen that the activity increased, and continued at a notably higher rate than pre-administration for 1000 seconds of tolbutamide administration. FIGS. 20 and 21, in combination, therefore demonstrate that the electrical activity measured by the electrode described hereinabove with reference to FIG. 18 is indeed endocrine in origin, and not due to other causes (e.g., gastrointestinal, neuronal, respiratory, electromyographic, or cardiac electrical activity). It is noted that these results are repeatable in many rats (at least 10) and were achieved in two different labs, by two different operators using different systems.

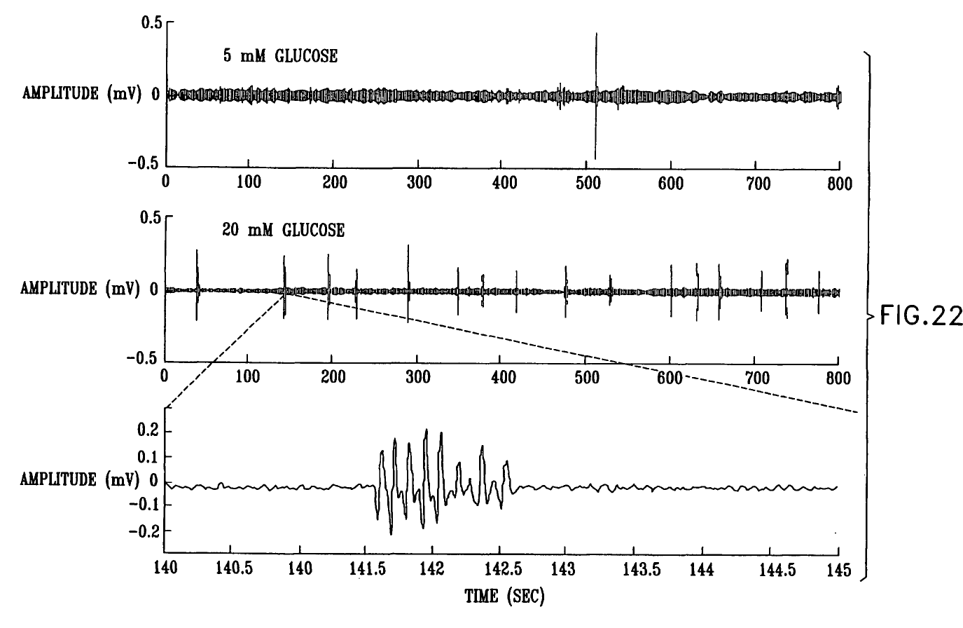

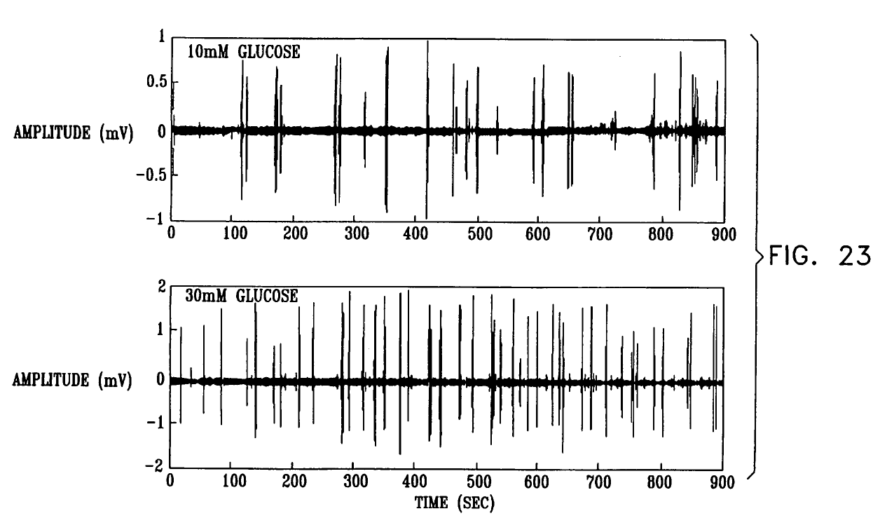

FIGS. 22 and 23 establish a strong correlation between the measured electrical activity and glucose level. The upper trace in FIG. 22 shows the minimal pancreatic electrical activity in an in situ rat at a low glucose level (5 mM), and the middle trace shows the significantly increased pancreatic electrical activity at a high glucose level (20 mM). An expanded view of one of the bursts from the middle trace is shown in the lower trace of FIG. 22.

The upper trace of FIG. 23 depicts the electrical activity in a different in situ rat experiment, this rat having an imposed normal-high glucose level of 10 mM. (The normal blood glucose level of a rat is approximately 8 mM.) The lower trace of FIG. 23 shows measured pancreatic electrical activity in response to a very high imposed glucose level—30 mM. Again, an increase in rate of bursts is detected.

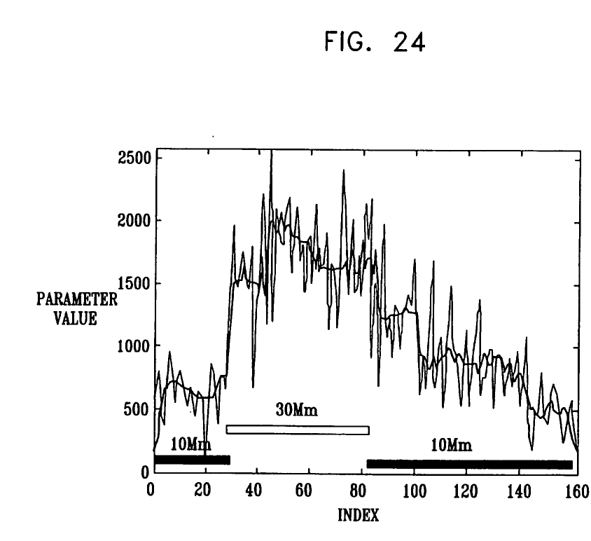

FIG. 24 shows the results of an experimental protocol in which a 10 mM glucose solution was perfused through a rat, then changed to a 30 mM solution, and then reduced once again to 10 mM. In analysis performed on the recorded electrical signals from this experiment, a “parameter value” based on the average amplitude of the spikes in the recorded bursts was calculated, and plotted against an index based on burst number. It is seen that there is a significant increase in the parameter value when the glucose level increases, and a corresponding dramatic decrease in the value when glucose level decreases back again to 10 mM. In a preferred embodiment of the present invention, control unit 90 determines a change in glucose level responsive to a change in an average amplitude of spikes in recorded bursts. Alternatively or additionally, the control unit analyzes other parameters (e.g., burst duration, average width (duration) of the spikes in a burst, changing frequencies of spikes within a burst, number of spikes per burst) to determine changes in glucose levels.

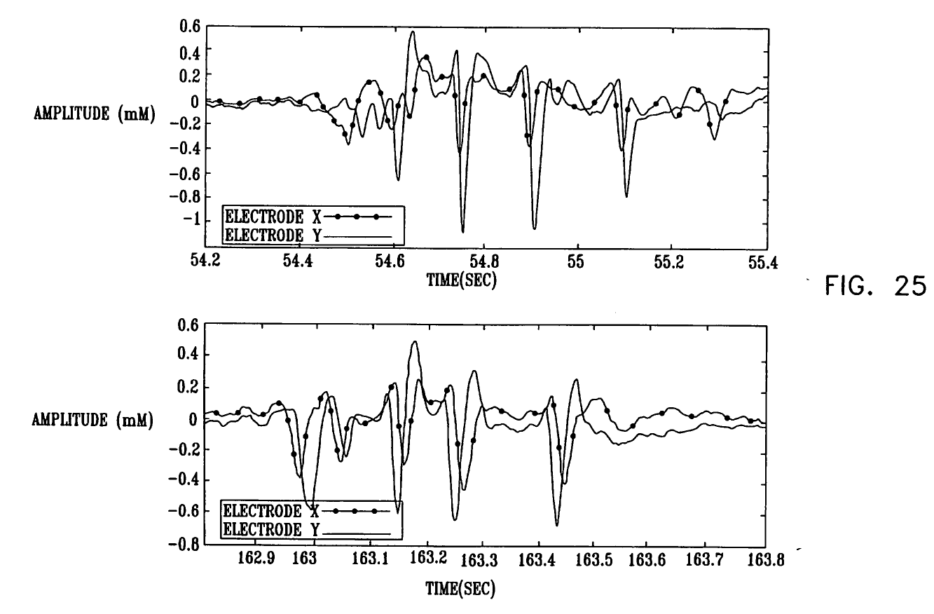

FIGS. 25, 26, and 27 demonstrate a level of synchronization between various pancreatic sites where electrical activity was measured. It was found that the electrical activity in normal rats at various sites is synchronized, and the inventors hypothesize that the synchronization is mediated at least in part by the blood stream and/or a central mechanism which governs the electrical activity of the pancreas (analogous to physiological pacemaker functioning in the heart). In both the upper trace and in the lower trace of FIG. 25, readings are shown from two electrodes (“X” and “Y”), placed on the pancreas approximately 1-2 cm apart. Reference and ground electrodes were common for electrode X and electrode Y. In the upper trace, it is seen that there is a delay between the two traces, in particular, that each of the four dominant downward spikes recorded by electrode Y is very shortly preceded by a downward spike recorded by electrode X. In the lower trace, by contrast, some of the downward spikes recorded by electrode Y were followed by a downward spike by electrode X, while others of the spikes were preceded by a downward spike recorded by electrode X.

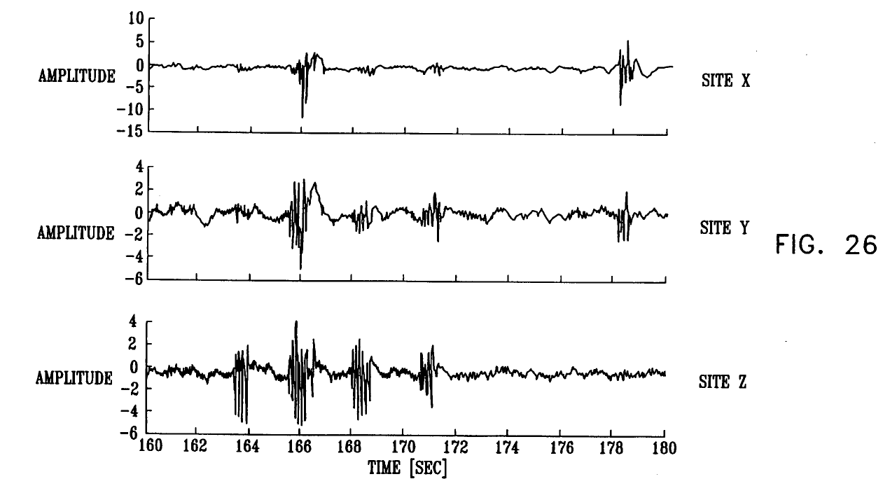

FIG. 26 depicts recordings from three pancreatic sites X, Y, and Z, spaced approximately 2 cm apart. In the three traces it can be seen that sometimes burst activity is detected at one or more of the sites, but not at another one of the sites (e.g., at T=164, activity is essentially limited to site Z, while at T=168.5, activity is seen at sites Y and Z).

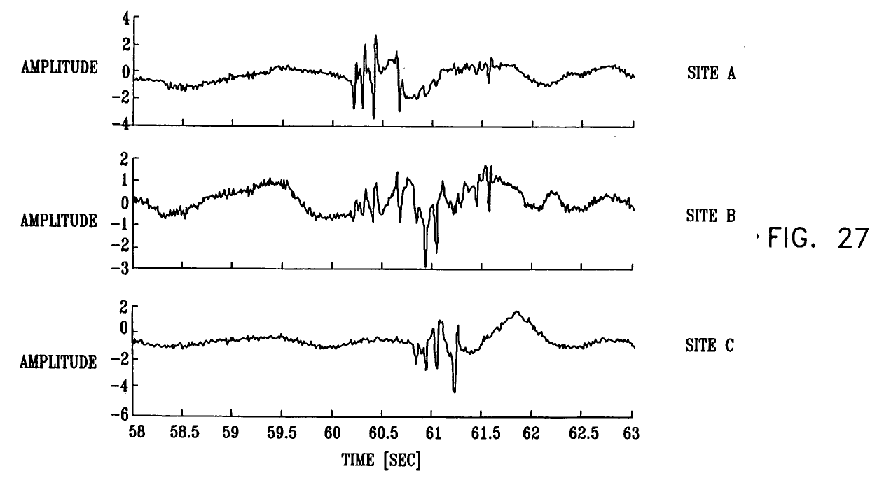

FIG. 27 shows differences in the lengths and onset times of bursts, based on the sites where the bursts are detected. For example, the burst at site B is simultaneous with but longer than that at site A, which in turn precedes (and may be longer than) that at site C. The inventors hypothesize that at a given point in time, some islets are active while other islets are silent. A degree of synchronicity is preferably determined according to the relative active number of islets in the area of the recording electrode. For some applications, a stimulus may be applied to cause the silent islets to depolarize, thereby typically increasing the synchronicity between various pancreatic sites and/or causing “recruitment” of a plurality of islets. Alternatively, the stimulus may be configured to reduce insulin secretion. The inventors believe that for some patients, increasing synchronicity (i.e., more cells in their active/depolarization phase) correspondingly increases insulin secretion.

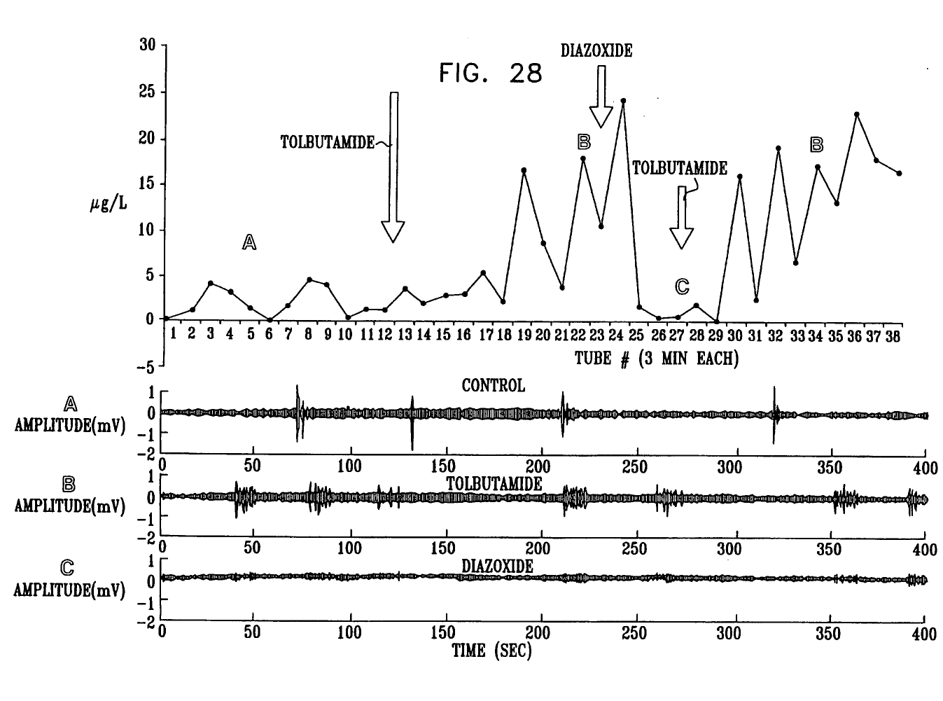

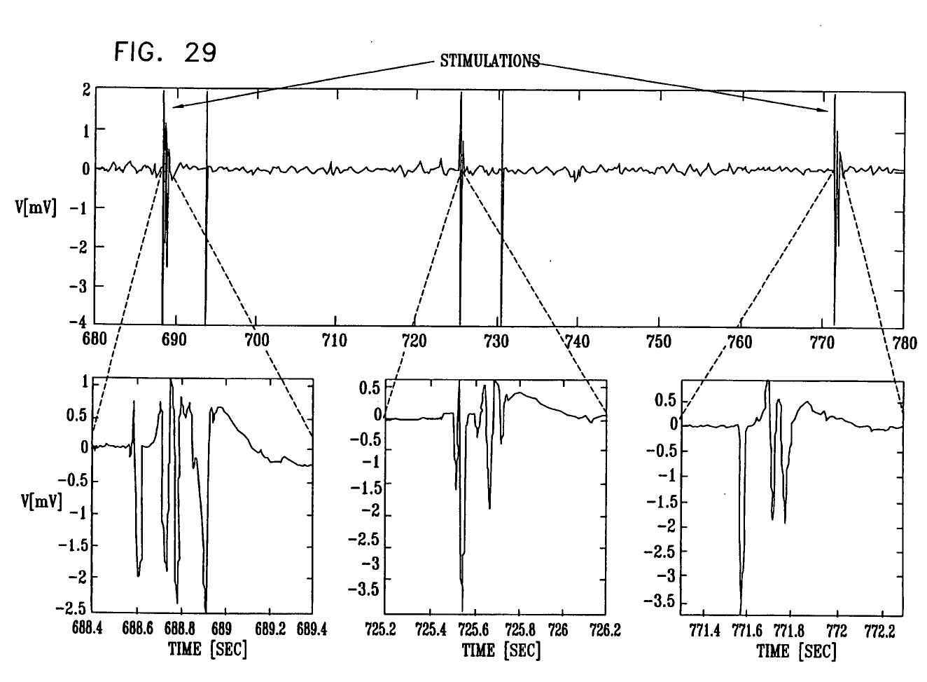

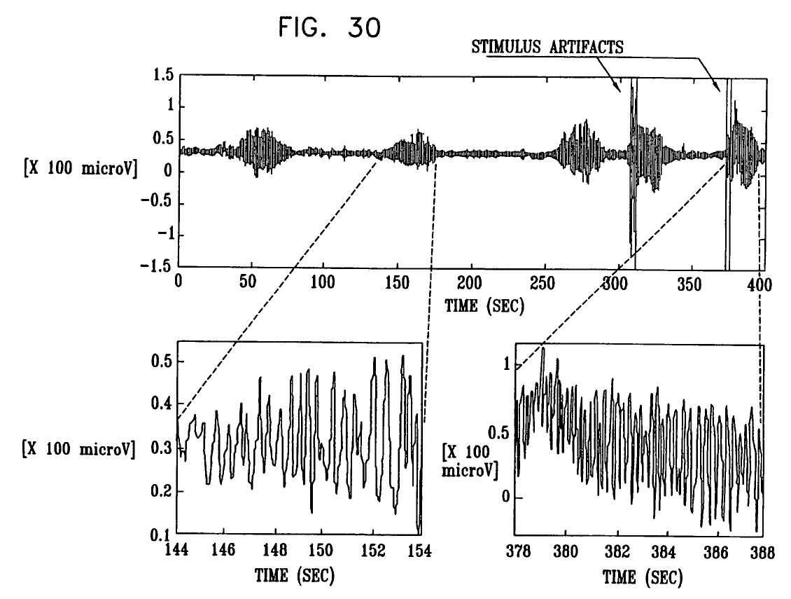

FIG. 28 depicts the correlation between measured pancreatic electrical activity and insulin secretion by the in situ pancreas. Insulin measurements were performed every three minutes for two and a half hours, which included an initial baseline period, a first tolbutamide administration period, a Diazoxide administration period, and a second tolbutamide administration period. During the initial baseline period, electrical activity was recorded during a 400 second baseline electrical measurement period A (FIG. 28, electrical trace labeled “Control”), and showed general electrical silence, interrupted at four points by short bursts.

Tolbutamide was administered after the twelfth sample was collected, and insulin measurements showed a clear trend of increase for the next ten samples (until Diazoxide was administered). A corresponding clear increase in the rate and duration of bursts is seen during the tolbutamide administration period. Subsequent administration of Diazoxide induces a complete inhibition of measured pancreatic electrical activity, and the measured levels of secreted insulin dropped at least to baseline levels, or to lower than baseline levels. During subsequent tolbutamide administration, additional increases in insulin secretion levels were detected, and these were accompanied by corresponding increases in electrical activity.

FIG. 29 shows the effects of stimulating the pancreas in accordance with a preferred embodiment of the present invention. Data shown in the present patent application are suggestive of a pancreatic mechanism which is analogous to the refractory period mechanism in the heart. FIG. 29, for example, shows five stimulations which were administered to an in situ pancreas. (Each stimulation is represented by a vertical bar in the upper trace of FIG. 29.) No significant levels of natural electrical activity are detected in the pancreas during the entire period of time displayed in FIG. 29. The first stimulation induces an immediate burst, but a second stimulation 5 seconds later does not induce a burst. Approximately 40 seconds after the first stimulation, a third stimulation is applied, again inducing a burst. A fourth stimulus only 5 seconds after the third does not induce a burst. Finally, after another 40 seconds, a fifth stimulus is given, which induces a burst. Hence, it seems that stimulations applied too closely in time do not induce bursts. In a preferred embodiment of the present invention, stimulation signals are applied to the pancreas at least about 0.5 to about 20 seconds following a detected or induced burst.

FIG. 30 shows natural burst activity and the induction of new bursts in an isolated islet in response to applied electrical stimulations at approximately T=315 seconds and T=375 seconds. In the lower left trace, an expanded view of normal burst electrical activity is shown (i.e., without applied stimulus), and in the lower right trace, an expanded view of an induced burst is shown. It is clearly seen that the frequency of the induced activity is substantially higher than the frequency of the non-induced burst. In a preferred embodiment of the present invention, an analogous stimulation protocol is used in patients in whom a higher burst frequency is associated with higher insulin secretion.

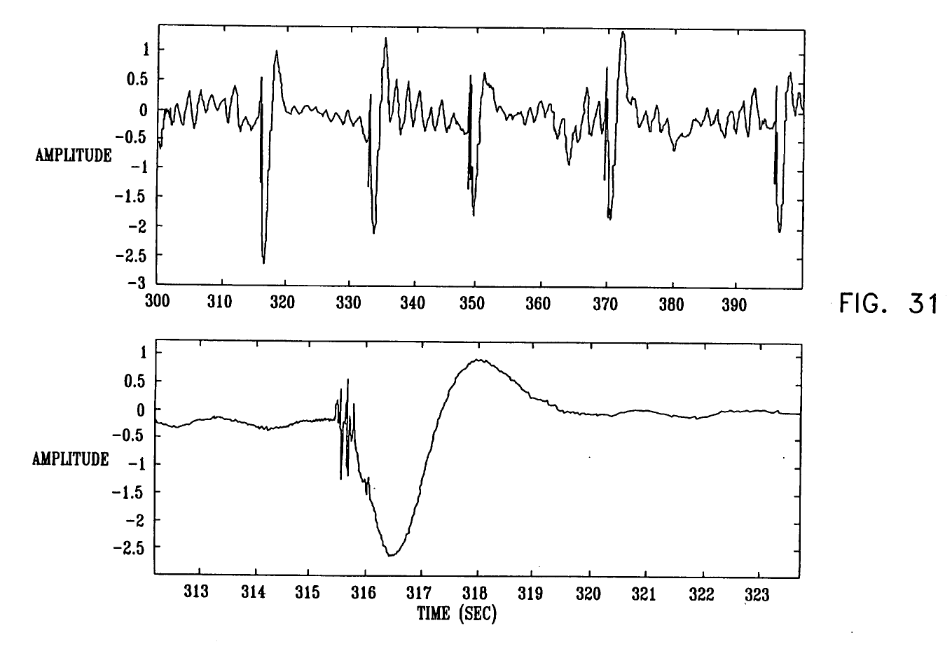

FIG. 31 shows pancreatic “slow waves,” which appear in synchrony with the burst activity, and which were measured in accordance with a preferred embodiment of the present invention. The upper trace shows 100 seconds of recorded pancreatic electrical activity, and the lower trace shows an expanded view of approximately twelve seconds from the upper trace, including a burst and a slow wave immediately thereafter. For some applications, these slow waves are analyzed by assuming that they are a summation of synchronized activity of islets at a relatively far distance from the recording electrodes. In analogy to ECG analysis, slow waves can be understood to be like an ECG signal, which represents the activity of an overall cell population, in contrast to being a recording of a local activity.

For some applications, a slow wave or burst is detected, and a stimulus is applied at a specified time after the onset of the slow wave or burst (e.g., during the slow wave or burst, or after the slow wave or burst), in order to enhance or otherwise modulate insulin secretion. For example, the stimulus may be applied 0-1 ms, 1-10 ms, 10-100 ms, 100-1000 ms, or 1-10 seconds after the onset of the slow wave or burst. For some applications, because of the pancreatic refractory periods described hereinabove with reference to FIG. 29, such a synchronized stimulus does not induce an extra slow wave or burst, but instead enhances or otherwise modulates a measure of overall pancreatic electrical activity, e.g., burst amplitude, duration, or frequency, and correspondingly increases or decreases insulin secretion.

Alternatively or additionally, sensing of pancreatic electrical activity is performed even with only one electrode, and an artificial stimulus is applied each time that a burst or slow wave is detected. The inventors believe that this develops in some patients a feedback loop, whereby the pancreas responds to elevated blood glucose by increasing its electrical activity (and increasing insulin secretion), and the stimulus applied to the pancreas further increases the insulin secretion, thereby supporting the pancreas in its effort to restore proper blood sugar levels. As blood sugar decreases, pancreatic electrical activity decreases and applied stimuli are consequently reduced.

It is hypothesized that a pancreatic equivalent of cardiac pacemaker cells may be responsible for controlling a significant portion of the slow wave or burst activity. In a preferred embodiment, a plurality of electrodes are placed at various sites on a patient's pancreas, and are driven in various sequences, using optimization algorithms known in the art, so as to determine a particular subset of the electrodes which maximally stimulate or modulate the propagation of slow waves or burst activity in the pancreas. Preferably, this calibration takes approximately a month, and is performed in cooperation with other tests (e.g., blood sampling) so as to determine stimulation protocols which achieve and then maintain glucose and/or insulin levels within desired ranges. Alternatively or additionally, one or more of the electrodes may be driven to induce slow waves or burst activity even without identifying the pancreatic equivalent of pacemaker cells.

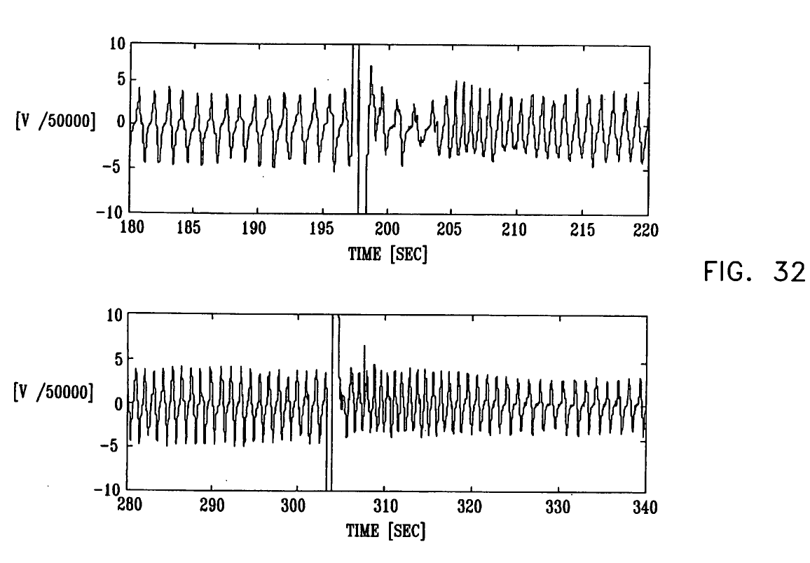

FIGS. 32-37 show modifications of the electrical activity of an isolated islet in response to an electrical stimulus applied in accordance with a preferred embodiment of the present invention. In the upper trace of FIG. 32, the stimulus applied at approximately T=197 seconds induces a decrease in activity until about T=204 seconds, followed by an increase between about T=205 and about 215 seconds, and a gradual return to normal activity. In the lower trace of FIG. 32, an initial increase in frequency in response to the applied stimulus is followed by a gradual reduction in frequency.

In the upper trace of FIG. 32, the stimulus induces an increase in frequency, followed by a decreased frequency associated with decreased signal magnitude, and, approximately a minute after application of the stimulus, a gradual return towards pre-stimulus frequency and magnitude. The lower trace of FIG. 32 shows an increase in frequency following a first stimulus, no change in frequency following a second stimulus applied 15 seconds later, and a gradual return to pre-stimulus frequency over the course of 1 to 1½ minutes.

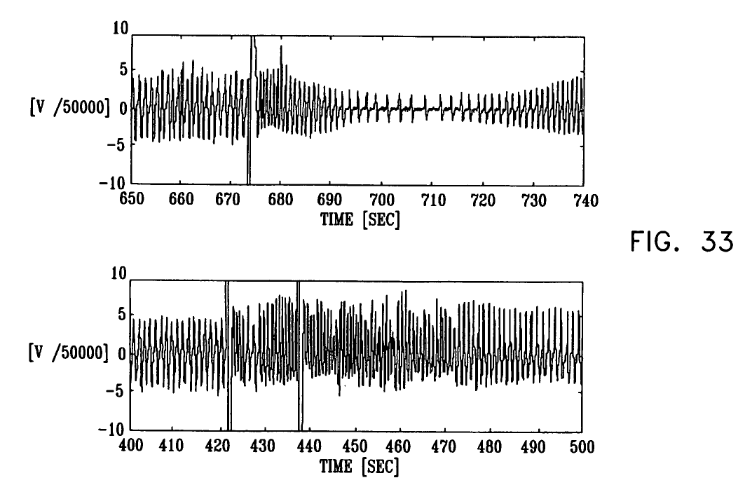

In the upper trace of FIG. 33, an increase in frequency immediately following the applied stimulus (at approximately 674 seconds) is followed shortly thereafter by a gradual return to pre-stimulus frequency within approximately ten seconds. Thereafter, a decrease in frequency for approximately 40 seconds is followed by a gradual increase in frequency towards baseline. In the lower trace of FIG. 33, an increase in frequency immediately following a first applied stimulus (at approximately 422 seconds) is sustained until the application of a second applied stimulus (at approximately 438 seconds). After the second stimulus, the increased frequency continues for approximately another 25-30 seconds, after which a return to approximately baseline is seen.

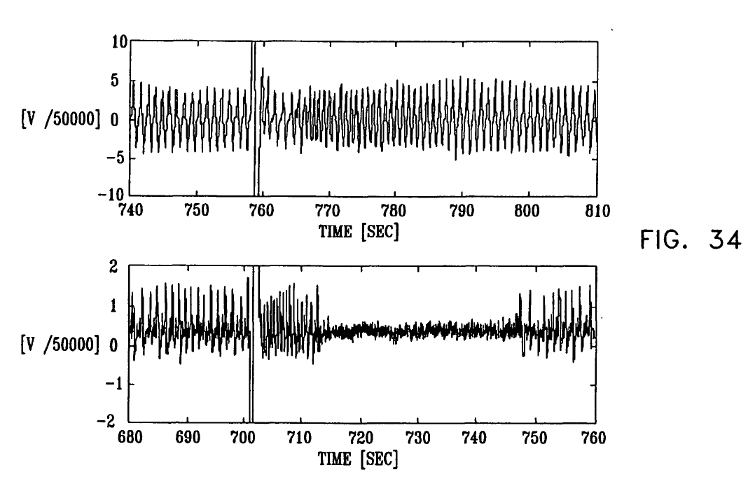

In the upper trace of FIG. 34, a decrease in frequency immediately following the applied stimulus (at approximately 758 seconds) is followed shortly thereafter by an increase in frequency, and a gradual return to pre-stimulus frequency within approximately 30 seconds. In the lower trace of FIG. 34, the increase in rate following application of the stimulus (at approximately 702 seconds) is followed by an essentially complete cessation of activity for half a minute, after which the activity is resumed at the pre-stimulus frequency and magnitude.

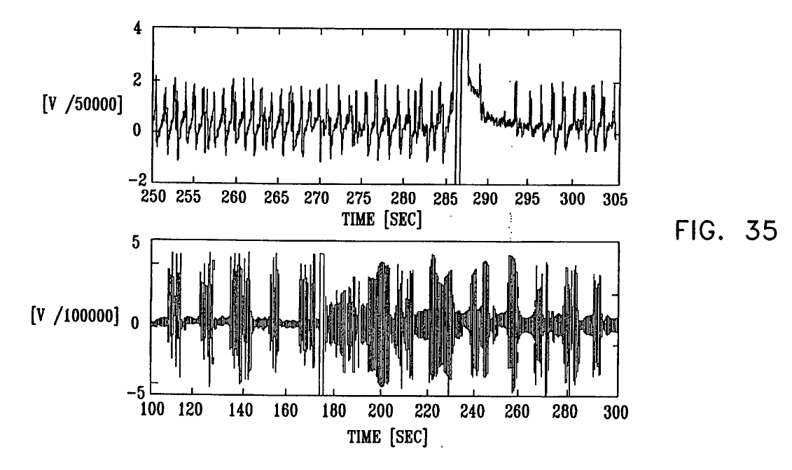

In the upper trace of FIG. 35, activity is seen to essentially cease for approximately 5 seconds following application of the stimulus, but to then resume several seconds thereafter. In the lower trace of FIG. 35, the applied stimulus induces a burst, which is of much greater duration than typical non-induced bursts. Pre-stimulus electrical activity is restored following the extra-long induced burst.

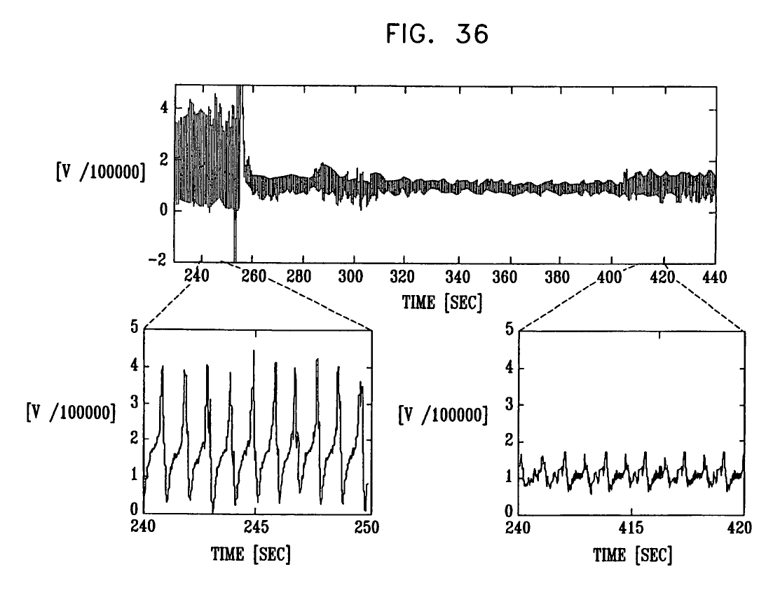

In the upper trace of FIG. 36, activity is effectively stopped in response to the applied stimulus, but then resumes after two minutes with an amplitude lower than pre-stimulus.

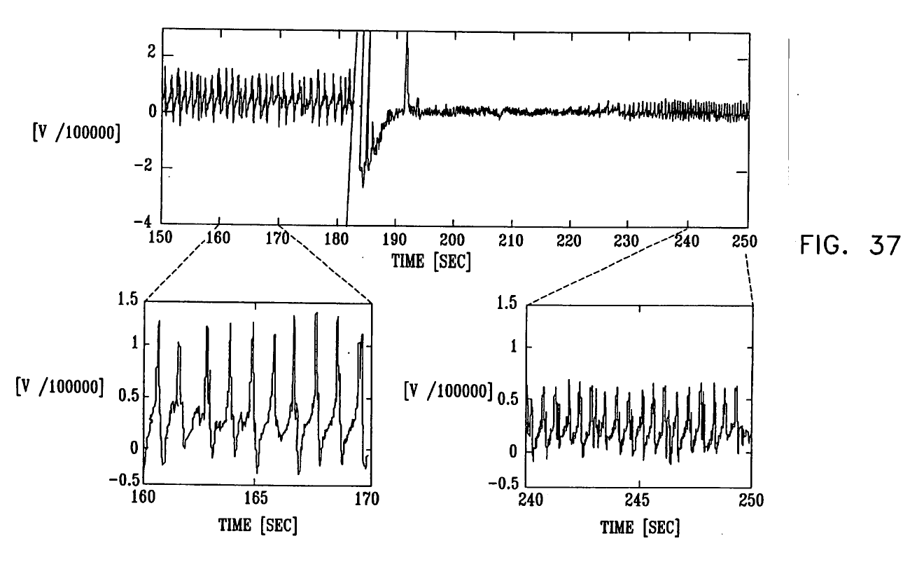

In the upper trace of FIG. 37, activity is seen to stop in response to the applied stimulus, and to resume with a lower amplitude than pre-stimulus after approximately one minute. The responses seen in FIGS. 36 and 37 are hypothesized to result from a smaller number of cells and/or islets which are electrically active.

FIGS. 32-37 thus show several examples of the types of pancreatic responses which can be induced in response to an applied stimulus. For clinical applications, a calibration period such as that described hereinabove is preferably provided for each patient, to determine for that patient suitable stimulation parameters which induce desired changes in insulin levels. It is noted that for patients for whom a high rate of islet activity is correlated with an increase in insulin secretion (an in situ example of which is shown hereinabove), FIGS. 32-37 show that a stimulus can be applied to increase or decrease insulin secretion.

For some applications, the need to increase or decrease insulin secretion can be satisfied by reversing the polarity of the applied stimulus. Alternatively or additionally, other parameters, such as magnitude, duration, or frequency of the applied stimulus can be modified to achieve a desired change in insulin secretion.

In a preferred application, the applied stimulus includes a square wave between approximately several tens of microamps to several milliamps (or higher, depending on electrode configuration), has a frequency between about 1 and about 500 Hz, and a delay from the start of a burst or slow wave of about 0 to about 1 second. The duration of the signal is typically either (a) the width of a single pulse or (b) between about 50 ms and about 1 second.

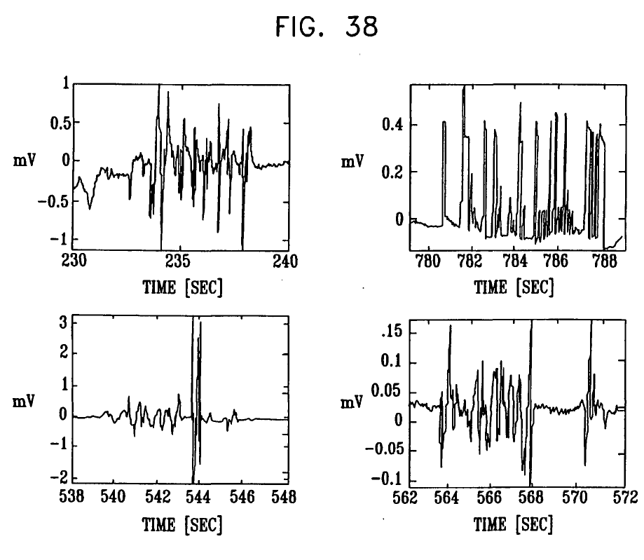

FIG. 38 shows pancreatic electrical activity recorded by electrodes sutured to connective tissue of the pancreas of a live pig (but not to the pancreas itself), in accordance with a preferred embodiment of the present invention. In this procedure, a small portion of the connective tissue that surrounds the pancreas was peeled back to create a pocket. An electrode was inserted into the pocket, so as to be touching the pancreas but sutured to the connective tissue. This technique was found to generally avoid injury to the pancreas, and is believed by the inventors to be suitable for long-term use in humans, as the pig pancreas is generally anatomically similar to that of a human. Signals were recorded for three hours using this technique without any noticeable deterioration. After three hours, electrical recording was discontinued.

It is also noted that the inventors have successfully sutured electrodes directly to a pig pancreas, and after a week no tissue rupture or dramatic inflammation was visible (as would be expected if the exocrine pancreas were damaged). Any of the surgical techniques described herein may typically be performed laparoscopically or using other known surgical methods.

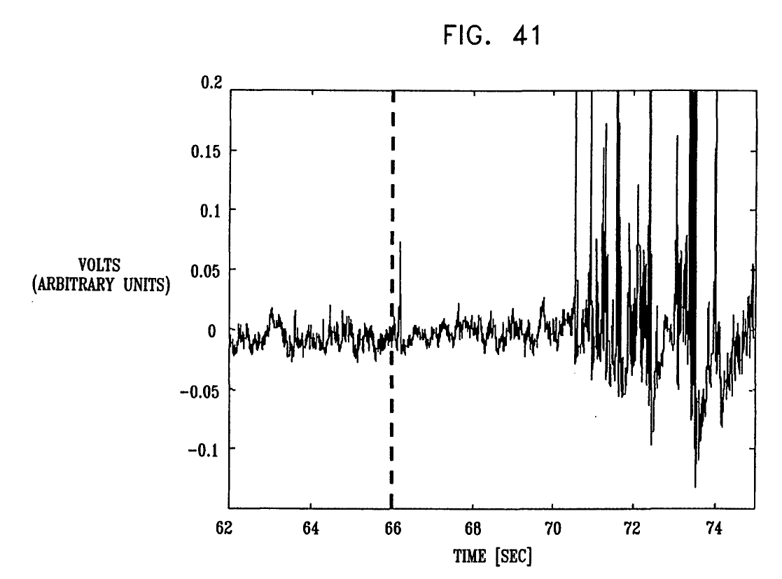

FIGS. 41, 42, and 43 are graphs showing in vivo experimental results, measured in accordance with a preferred embodiment of the present invention. A Sinclair minipig was pre-anesthetized with Acepromazine and Ketamine, and was anesthetized with 1-2% Isoflurane. A midlaparotomy was performed about 15-about 20 cm below the sternum. The pancreas was exposed by means of an abdominal retractor. Three single-electrode patch assemblies similar to those described with reference to FIG. 3B were carefully attached to the body and the tail of the pancreas, and were kept in place using a non-absorbable, multi-filament suture. A single 25× signal preamplifier (Analog Devices 620 BR 0128, 3 Technology Way, Norwood, Mass., USA), and a 50× amplifier, attached on the top of the patch assembly, were both used. The left external jugular vein was exposed and a catheter was inserted and tunneled to the intra-scapular space, to allow drug or glucose injections, and to allow blood samples to be taken for glucose and insulin concentration measurements. The electrical connector and the cannula were covered with adhesive bandages in order to prevent the minipig from damaging them. The minipig was given analgesics and antibiotics for a 3-15 day recovery period after surgery. The minipig was free to walk around while measurements were taken. Leads used included both mono-polar, temporary cardiac pacing wires (A&E Medical Corporation) and bipolar temporary myocardial pacing leads (Medtronic, Inc.). Although not tested in this series of experiments, for some applications blood glucagon level is alternatively or additionally tested. To exclude the effect of mechanical artifacts, the minipig was placed alone in a cage throughout the experiment, except during a 1.5-minute period during an injection of glucose, as described below. Additionally, movements of the minipig were manually recorded.

Additionally, electrical impedance between two sites on the stomach was measured, by placing two wire electrodes therein, in order to facilitate a determination of the effect of motion of the stomach on the pancreatic electrical activity measurements. Two similar electrodes were placed on the pancreas to detect changes in pancreatic electrical impedance across a distance, so as to detect movement of the pancreas. A correlation was found between the activity measurements and motion of the stomach and of the pancreas. In a preferred embodiment of the present invention, apparatus 18 comprises one or more stomach “impedance electrodes” (not shown), configured to sense stomach motion. Control unit 90 receives a signal indicative of a measure of stomach motion from the stomach impedance electrodes, and adjusts the recorded pancreatic signals responsive thereto, such as by using a subtraction algorithm.

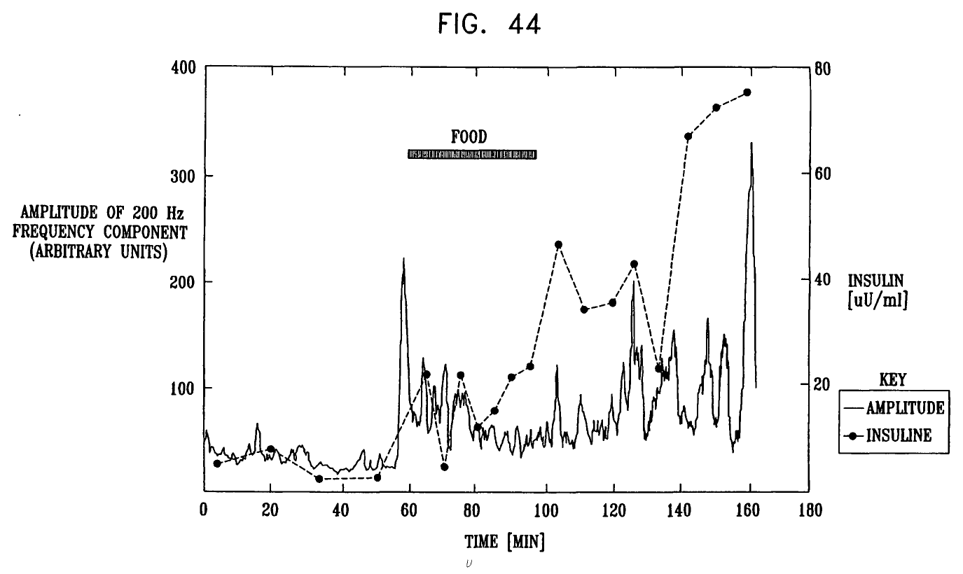

The wires of the electrodes (formed in a braid) were passed through the back of the minipig, under the skin of the left abdominal wall, and connected to an external device having a sensory channel. The external device was connected to a computer, which recorded signals sampled at between 0 and 500 Hz, and saved the recorded signals for off-line analysis. The analysis shown in FIG. 44 was performed using signals sampled at 200 Hz.

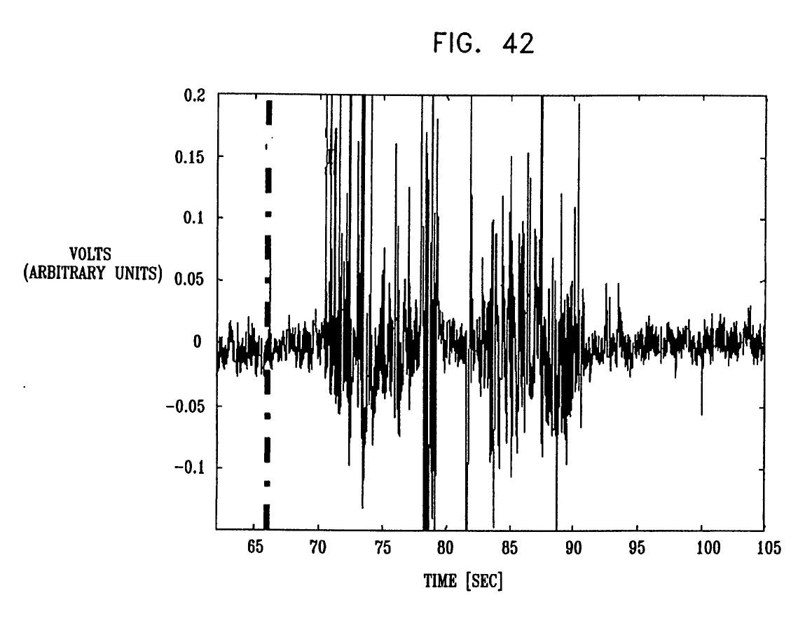

Readings from the pancreas were recorded during an hour-long period while the minipig was fasting, and without the administration of glucose or any drug. At minute 66 from the beginning of the recording, 30 cc of 50% dextrose was injected into the jugular vein. The injection was completed in 1.5 minutes. As is seen in FIG. 41, a strong response in the signal, indicated by a clear change in the amplitude of the signal, began approximately two minutes after the injection. As is seen in FIG. 42, which includes the information shown in FIG. 41 as well as information for a longer time period, this strong response continued for a period of about 20 minutes, after which the signal returned essentially to its baseline level.

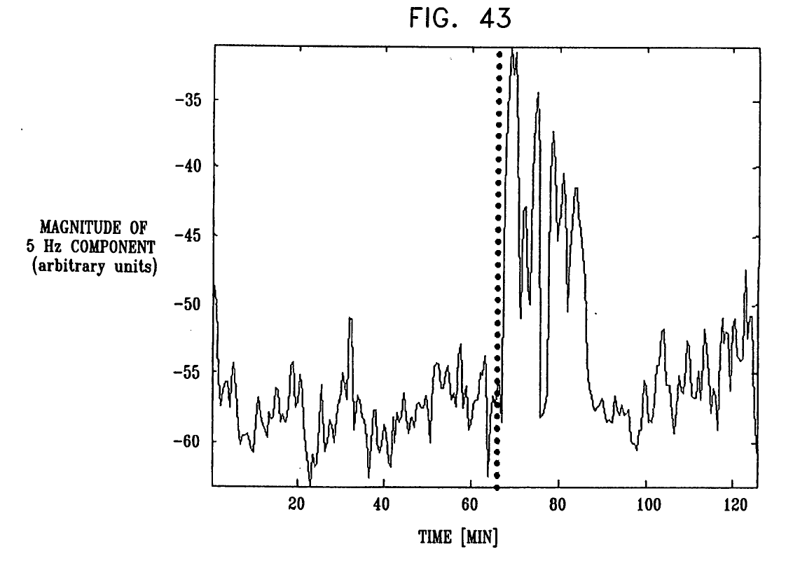

FIG. 43 shows an analysis of the raw signal, performed in accordance with a preferred embodiment of the present invention, reflecting the amplitude of the signal over time at a frequency of 5 Hz. It can be seen that there is an increase in the energy at this particular frequency in response to the injection of dextrose. In preferred embodiments of the present invention, changes in magnitude of one or more frequency components of the recorded pancreatic electrical signals are used as an indication changes in blood glucose and/or blood insulin levels.

FIG. 44 is a graph showing in vivo experimental results, measured and analyzed in accordance with a preferred embodiment of the present invention. The y-axis in this figure represents the magnitude of a calculated 10 Hz component of measured pancreatic electrical activity in a second minipig. The right jugular vein was cannulated to allow drug or glucose injections, and to allow blood samples to be taken for glucose concentration measurements. Three sets of electrodes were carefully attached to the pancreas: (a) a pair of pair of button electrodes, similar to those described hereinabove with reference to FIGS. 3C and 3D, (b) a concentric electrode, similar to those described hereinabove with reference to FIG. 3B, and (c) a patch with two wire electrodes similar to that described hereinabove with reference to FIG. 3A. FIG. 44 shows results generated using the wire electrode, as shown in FIG. 3A. Two preamplifiers, one providing amplification of 25× and the other of 50×, were used. Electronics attached to a separate patch were used.

The wires of the electrodes were passed through the back of the minipig and connected to an external device comprising sensor and delivery channels. The external device was connected to a computer, which recorded signals sampled at 0 to 500 Hz, and saved the recorded signals for off-line analysis. The analysis was performed using a sampling rate of 200 Hz.

Readings from the pancreas were recorded during an hour-long period while the minipig was fasting, and without the administration of glucose or any drug. From minute 60 to minute 98 from the beginning of the recording, the minipig was fed. As is seen in FIG. 44, a spike in the amplitude of the 10 Hz component of the measured signal occurred about approximately one minute before the minipig began to eat. This pre-eating response is attributed to the animal's knowledge of the imminent meal (food was placed in the animal's food basket). A strong response is seen beginning about 2 to about 3 minutes after the commencement of eating and continuing for a period of about 20 minutes, after which the signal began to return towards its baseline level. About 20 minutes after the minipig stopped eating, a second response began. This second response is attributed to digestion of the food, which causes an increase in glucose and insulin levels, in part dependent upon the specific composition of the food. Blood insulin levels were also measured. Beginning at approximately the commencement of eating, an increase in insulin level was observed. (The rise began immediately before ingestion, during the cephalic phase, when the minipig had seen the food and knew it was about to it.) During digestion of the meal, insulin levels continued to increase fairly rapidly, reaching about 75 uU/ml, compared to about 5 to about 10 uU/ml before eating. The increase in insulin level closely tracked the amplitude of the displayed 200 Hz frequency component.

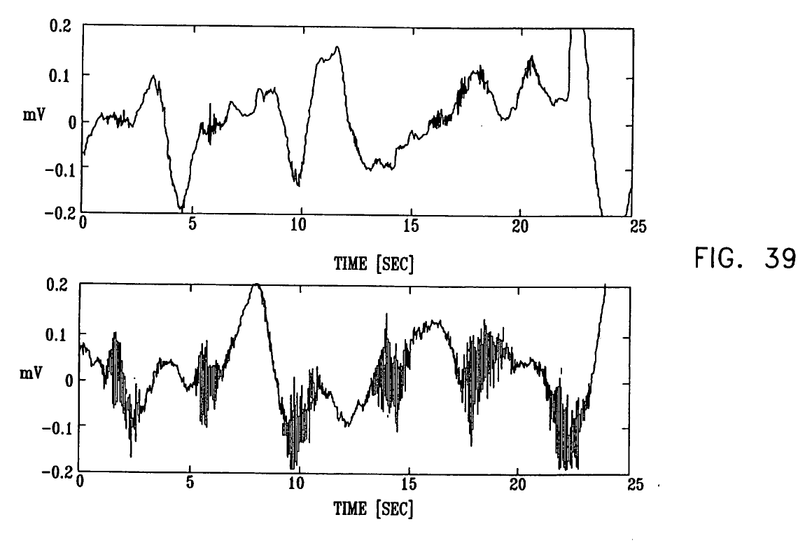

FIG. 39 is a graph showing in vivo experimental results, measured and analyzed in accordance with a preferred embodiment of the present invention. Wire electrodes were inserted into a minipig's pancreas. Leads connected to the wire electrodes extended out of the minipig to signal amplifiers located outside of the minipig. The upper trace shows baseline activity. It can be seen that periodic low-intensity bursts occurred, such as at about 2-3 seconds and at about 6 seconds. The lower trace shows electrical activity beginning about 115 seconds after an oral dose of glucose was administered. (The upper and lower traces were recorded during different time periods.) After administration of the glucose, the intensity of observed bursts increased markedly. The y-axis of the upper trace is on the same scale as the y-axis of the lower trace.

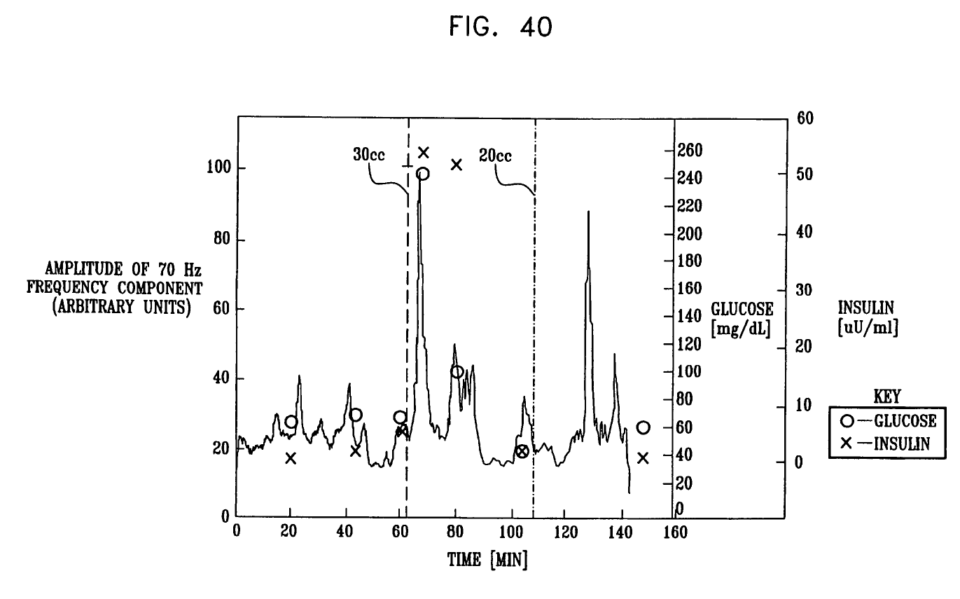

FIG. 40 is a graph showing in vivo experimental results, measured and analyzed in accordance with a preferred embodiment of the present invention. Button electrodes similar to those described hereinabove with reference to FIG. 3C were attached to the pancreas of a minipig. The electrodes were coupled to an amplifier fixed to a patch, which was also attached to the pancreas. The displayed data were recorded approximately 2 weeks post-surgery, in a conscious minipig free to walk around its cage. The trace shows the amplitude of the 70 Hz frequency component of the measured signal. Blood samples were periodically taken, and blood glucose (mg/dL) and blood insulin (uU/ml) levels were measured.

During the first approximately 64 minutes, electrical activity was relatively flat, and, correspondingly, glucose and insulin levels remained fairly steady. At approximately 64 minutes, 30 cc of 50% dextrose was administered intravenously. Within about 2 to about 3 minutes, a sharp spike in the magnitude of the 70 Hz frequency component was observed. At this point, blood glucose and insulin levels also jumped sharply. All three indicators of pancreatic activity gradually declined over the next approximately 35 minutes, at which point 20 cc of 50% dextrose was administered intravenously. In response to this lower dose, smaller spikes in the 70 Hz frequency component were observed, beginning at approximately 128 minutes. (Insulin and blood glucose samples were not collected at this point.) Blood glucose and insulin levels at about 150 minutes were very slightly lower than baseline levels. FIG. 40 shows a strong correlation between pancreatic electrical activity, as measured and analyzed using techniques of an embodiment of the present invention, and blood glucose and insulin levels, before, during, and after administration of intravenous glucose.

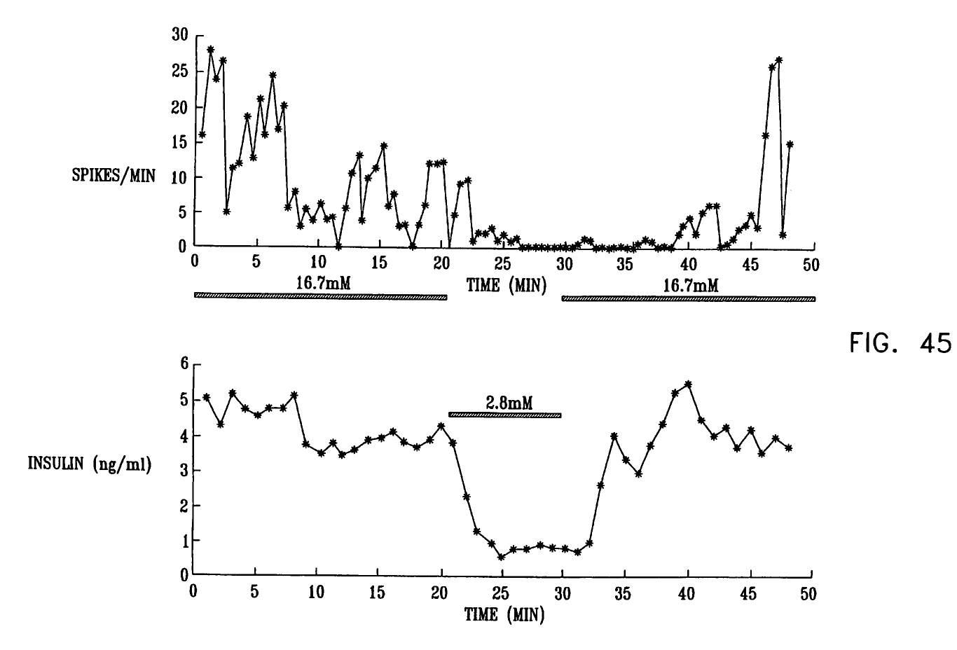

FIGS. 45, 46, and 47 are graphs showing in situ experimental results, measured in accordance with a preferred embodiment of the present invention. A Sprague Dawley rat was sacrificed and perfused through the descending aorta after the main blood vessels to the colon, kidney and gut were closed. Perfusate samples were collected from the portal vein using a fraction collector for insulin measurements. Electrical activity of the pancreas was recorded using patch electrodes such as those shown in FIG. 3A, coupled to the pancreas and connected to an amplifier.

FIG. 45 shows an analysis of the effect of blood glucose concentration on pancreatic electrical activity and insulin secretion, in accordance with a preferred embodiment of the present invention. Readings from the pancreas were recorded over a 48-minute period during which blood glucose concentration was tightly controlled via the concentration of the perfusate. During the first 20 minutes, perfusate glucose concentration was 16.7 mM. A relatively high rate of spike generation (spikes per minute) was seen during this period, corresponding to a relatively high level of insulin secretion, as measured by insulin concentration in the perfusate (of between about 3.5 to about 5 ng/ml). During a ten-minute period beginning at 20 minutes, perfusate glucose concentration was lowered to 2.8 mM. The rate of spike generation dropped sharply and remained low (nearly zero) throughout this period, corresponding to a recorded steep drop in insulin secretion over the first five minutes of this period, leveling off at about 1 ng/ml during the second five minutes of this period. In the remaining period of the experiment, beginning at 30 minutes, perfusate glucose concentration was increased back to 16.7 mM. After about ten minutes, the rate of spike generation began increasing, returning, after about 15 minutes from the beginning of this period, to a rate similar to that observed during the first period of the experiment. During this third period, insulin secretion began increasing at about two minutes into the period, returning, at about four minutes into the period, to a level similar to that observed during the first period. In a preferred embodiment of the present invention, a rate of spike generation is analyzed to determine a rate of insulin secretion and/or a blood glucose level.

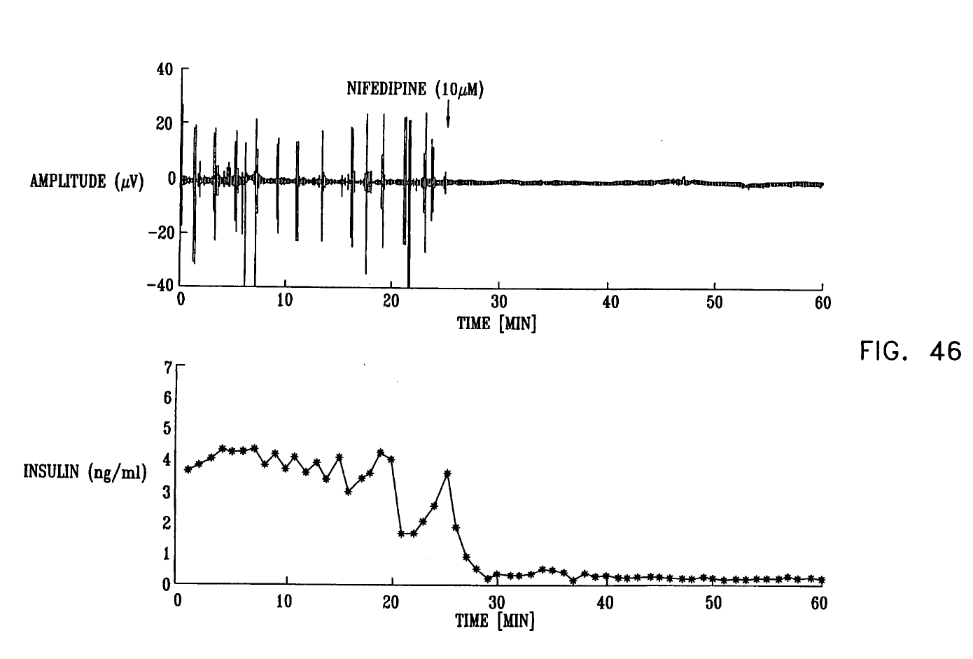

FIG. 46 shows the effect of administration of a calcium channel blocker on pancreatic electrical activity and insulin secretion, in accordance with a preferred embodiment of the present invention. Readings from the pancreas were recorded over a one-hour period. During approximately the first 24 minutes, a fairly constant normal magnitude of pancreatic electrical activity was observed, corresponding to a fairly constant level of insulin secretion. At about 24 minutes, Nifedipine (10 μM), a calcium channel blocker, was administered. A sudden drop in electrical activity and corresponding drop in insulin secretion was observed almost immediately.

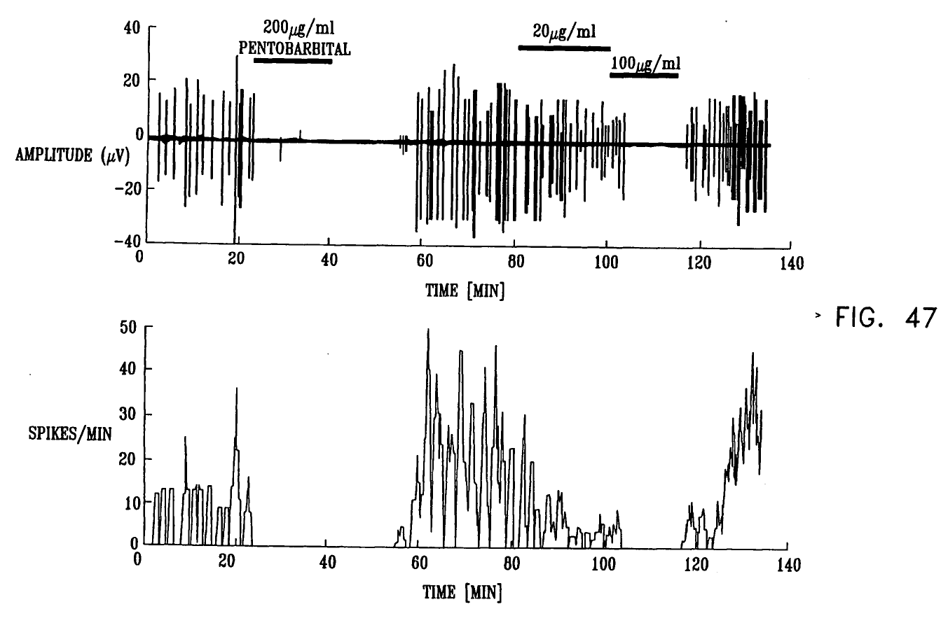

FIG. 47 shows the effect of anesthesia on pancreatic electrical activity, in accordance with a preferred embodiment of the present invention. Readings from the pancreas were recorded over about a 135-minute period. Normal levels of pancreatic electrical activity, as measured by the magnitude of the electrical signal and by the rate of spike generation, were observed during the first approximately 22 minutes. At this point, Pentobarbitone sodium (200 μg/ml) was administered, resulting in an almost complete block of pancreatic electrical activity, as seen in both the magnitude of the electrical signal and the rate of spike generation. Beginning at about 40 minutes, administration of the anesthesia was halted, resulting in a return at 58 minutes to activity levels somewhat higher than the levels seen in the first 22-minute period. At about 80 minutes, a lower concentration of Pentobarbitone sodium (20 μg/ml) was administered, which reduced burst frequency and the rate of spike generation, without producing the near total block seen during the period of administration of a 200 μg/ml concentration. Beginning at about 100 minutes, a concentration of 100 μg/ml was administered, resulting in a near total block beginning at about 103 minutes, and lasting until about 117 minutes, when the anesthesia was again halted. Electrical activity is seen resuming slightly after this point.

In a preferred embodiment of the present invention, signals generated by electrodes are analyzed using a moving window. Preferably, the duration of each window is between about 1 and about 300 seconds, and sequential windows overlap one another by about 20 to about 80 percent of the duration of each window. A Fourier transform or other transform is applied to the signal for the time period of each window, and the amplitude of each frequency component is stored. One or more algorithms are used to detect indications of clinically-significant phenomena, such as an increase in blood glucose and/or insulin levels from normal to elevated or supraphysiological values. Preferably, responsive to the outputs of one or more such algorithms, a decision is made regarding whether to apply a therapeutic response.

Preferably, the algorithms calculate one or more of the following:
substantial inter-window increases or decreases in the amplitude of frequency components between about 0 and about 100 Hz; and/or
changes in a ratio of (a) the amplitude of a frequency component from the high range of frequencies in the sampled data to (b) the amplitude of a frequency component in the low range of frequencies.

Alternatively or additionally, algorithms are used in order to identify one or more of the following:
patterns in the frequency domain of the Fourier transform;
patterns in the time domain of the data, prior to application of the Fourier transform; and/or
zero-crossings.

Preferably, interference caused by non-pancreatic electrical activity sensed by the electrodes is reduced using one or more of the following methods:
When an array of electrodes is applied to the pancreas, the known or calibrated delay between different areas of activity on the pancreas is used to determine whether each signal is caused by pancreatic activity.
One or more electrodes are used to detect mechanical artifacts that are more clearly detectable and distinguishable in one area of the pancreas in the vicinity of such electrodes than in the vicinity of other areas of the pancreas. For example, the effect of mechanical artifact due to motion of the stomach or duodenum may be reduced in this manner.
Mechanical artifacts are identified by distinguishing spectral patterns or time patterns thereof, and removed from the signal.
Direct measurements are made of physiological or non-physiological phenomena which are expected to provide some level of interference. These measurements serve as inputs to noise-reduction algorithms that minimize the effect of the measured phenomena from the pancreatic electrical signal. For example, ECG measurements, respiration measurements, or body acceleration measurements may be used as inputs to the noise-reduction algorithms.

For some applications, it is desirable to increase current density applied to the pancreas or associated connective tissue to a relatively high value, e.g., by driving 1-20 mA (preferably 5 mA) through an electrode having an area of 0.001 cm2 to 1 cm2 (preferably approximately 0.005 cm2).

It is to be understood that whereas preferred embodiments of the present invention are described with respect to sensing and/or stimulating a patient's natural pancreas, some of the same techniques may be adapted for sensing and/or stimulating implanted islets or beta cells, so as to regulate a patient's glucose and insulin levels. It is also to be understood that “magnitude” and “amplitude,” as used in the specification and the claims, are synonymous.

It is to be further understood that whereas preferred embodiments of the present invention are described with respect to sensing pancreatic electrical activity, similar measurements may be made, alternatively or additionally, of oscillations in calcium levels and/or oscillations in other pancreatic functions, e.g., pancreatic metabolic function, and analyzed, mutatis mutandis, to yield an indication of blood glucose and/or insulin level. For example, one or more calcium electrodes may be coupled to various sites on a patient's pancreas and activated to yield indications of intracellular or interstitial calcium levels. Alternatively or additionally, dyes or other indicators of calcium or ATP/ADP conversion may be used to indicate pancreatic functioning, for example, in combination with implanted light sources and/or detectors.

It is also to be understood that when, for example, electrodes 100 are described herein as “generating” an activity signal, this comprises recording electrical activity and conveying an activity signal, responsive thereto, to an element that receives the activity signal (e.g., signal amplification and processing circuitry).

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

1: Apparatus for sensing electrical activity of a pancreas of a patient, comprising:
a set of one or more electrodes, adapted to be coupled to the pancreas, and to generate activity signals indicative of electrical activity of pancreatic cells which are in a plurality of islets of the pancreas; and
a control unit, adapted to receive the activity signals, and to generate an output signal responsive thereto.

2: Apparatus according to claim 1, wherein a single electrode in the set of one or more electrodes is adapted to convey to the control unit an activity signal indicative of electrical activity of pancreatic cells which are in two or more of the islets.

3: Apparatus for analyzing electrical activity of a pancreas of a patient, comprising:
a set of one or more electrodes, each electrode adapted to be coupled to the pancreas and to generate an activity signal indicative of electrical activity of pancreatic cells which are in a plurality of islets of the pancreas; and
a control unit, adapted to:
receive the activity signals from the one or more electrodes,
analyze the received activity signals, and
generate an output signal responsive to the analysis.

4: Apparatus according to claim 3, wherein the set of electrodes is adapted to generate activity signals indicative of electrical activity of pancreatic cells which are in five or more of the islets.

5: Apparatus according to claim 3, wherein the set of electrodes is adapted to generate activity signals indicative of electrical activity of pancreatic cells which are in ten or more of the islets.

6: Apparatus according to claim 3, wherein a first one of the one or more electrodes is adapted to generate a first activity signal, indicative of electrical activity of pancreatic cells which are in a first one of the islets, and wherein a second one of the one or more electrodes is adapted to generate a second activity signal, indicative of electrical activity of pancreatic cells which are in a second one of the islets, which is different from the first one of the islets, and wherein the control unit is adapted to receive the first and second activity signals.

7: Apparatus according to claim 3, wherein the control unit is adapted to analyze the activity signals so as to identify an aspect thereof indicative of activity of a type of cell selected from the list consisting of: pancreatic alpha cells, pancreatic beta cells, pancreatic delta cells, and polypeptide cells, and wherein the control unit is adapted to generate the output signal responsive to identifying the aspect.

8: Apparatus for monitoring a blood glucose level of a patient, comprising:
a set of one or more electrodes, adapted to be coupled to a pancreas of the patient, and to generate respective activity signals indicative of spontaneous electrical activity of pancreatic cells; and
a control unit, adapted to receive the respective activity signals, to analyze the activity signals so as to determine a change in the glucose level, and to generate an output signal responsive to determining the change.

9: Apparatus for monitoring a blood insulin level of a patient, comprising:
a set of one or more electrodes, adapted to be coupled to a pancreas of the patient, and to generate respective activity signals indicative of spontaneous electrical activity of pancreatic cells; and
a control unit, adapted to receive the respective activity signals, to analyze the activity signals so as to determine a change in the insulin level, and to generate an output signal responsive to determining the change.

10: Apparatus according to claim 8, wherein the control unit is adapted to analyze the activity signals so as to identify an aspect thereof indicative of activity of a type of cell selected from the list consisting of: pancreatic alpha cells, pancreatic beta cells, pancreatic delta cells, and polypeptide cells, and wherein the control unit is adapted to generate the output signal responsive to identifying the aspect.

11: Apparatus according to claim 3, wherein the control unit is adapted to analyze the activity signals so as to identify a frequency aspect thereof, and to generate the output signal responsive to identifying the frequency aspect.

12: Apparatus for analyzing electrical activity of a pancreas of a patient, comprising:
a set of one or more electrodes, adapted to be coupled to the pancreas, and to generate activity signals; and
a control unit, adapted to receive the activity signals, adapted to analyze the activity signals so as to identify an aspect thereof which is indicative of activity of pancreatic alpha cells, and adapted to generate an output signal responsive to identifying the aspect.

13: Apparatus for analyzing electrical activity of a pancreas of a patient, comprising:
a set of one or more electrodes, adapted to be coupled to the pancreas, and to generate activity signals; and
a control unit, adapted to receive the activity signals, adapted to analyze the activity signals so as to identify an aspect thereof which is indicative of activity of pancreatic beta cells, and adapted to generate an output signal responsive to identifying the aspect.

14: Apparatus according to claim 13, wherein the control unit is adapted to analyze the activity signals so as to distinguish between the aspect thereof which is indicative of the activity of the beta cells and an aspect thereof which is indicative of activity of pancreatic alpha cells, and wherein the control unit is adapted to generate the output signal responsive to distinguishing between the aspects.

15: Apparatus for analyzing electrical activity of a pancreas of a patient, comprising:
a set of one or more electrodes, adapted to be coupled to the pancreas, and to generate activity signals; and
a control unit, adapted to receive the activity signals, adapted to analyze the activity signals so as to identify an aspect thereof which is indicative of activity of pancreatic delta cells, and adapted to generate an output signal responsive to identifying the aspect.

16: Apparatus for analyzing electrical activity of a pancreas of a patient, comprising:
a set of one or more electrodes, adapted to be coupled to the pancreas, and to generate activity signals; and
a control unit, adapted to receive the activity signals, adapted to analyze the activity signals so as to identify an aspect thereof which is indicative of activity of polypeptide cells, and adapted to generate an output signal responsive to identifying the aspect.

17: Apparatus according to claim 13, wherein the control unit is adapted to compare the aspect of the activity signals with a stored pattern that is indicative of activity of the cells, and to generate the output signal responsive thereto.

18: Apparatus according to claim 13, wherein the control unit is adapted to analyze the activity signals under an assumption that the activity of the cells is dependent on electrical activity of another type of pancreatic cell, and to generate the output signal responsive thereto.

19: Apparatus according to claim 13, wherein the control unit is adapted to analyze the activity signals under an assumption that the activity of the cells is substantially independent of electrical activity of another type of pancreatic cell, and to generate the output signal responsive thereto.

20: Apparatus according to claim 13, wherein the control unit is adapted to analyze the activity signals so as to identify a frequency aspect thereof, and to generate the output signal responsive to identifying the frequency aspect.

21: Apparatus according to claim 20, wherein the control unit is adapted to analyze the activity signals so as to differentiate between a first frequency aspect of the activity signals which is indicative of the activity of the cells, and a second frequency aspect of the activity signals, different from the first frequency aspect, which is indicative of activity of another type of pancreatic cell.

22: Apparatus according to claim 20, wherein the control unit is adapted to analyze the activity signals so as to identify over time a change in the frequency aspect that is characteristic of the cells.

23: Apparatus according to claim 20, wherein the control unit is adapted to analyze the activity signals so as to identify a magnitude aspect thereof, wherein the control unit is adapted to analyze the frequency aspect and the magnitude aspect in combination, and wherein the control unit is adapted to generate the output signal responsive to analyzing the aspects.

24: Apparatus according to claim 20, wherein the control unit is adapted to analyze the activity signals so as to identify a duration aspect thereof, wherein the control unit is adapted to analyze the frequency aspect and the duration aspect in combination, and wherein the control unit is adapted to generate the output signal responsive to analyzing the aspects.

25: Apparatus according to claim 3, wherein the set of electrodes is adapted to generate the activity signals responsive to spontaneous electrical activity of the pancreatic cells.

26: Apparatus according to claim 3, wherein the control unit is adapted to apply a synchronizing signal to the pancreas.

27: Apparatus according to claim 3, wherein the control unit is adapted to analyze the activity signals so as to identify a magnitude of a fluctuation of the activity signals, and to generate the output signal responsive to the analysis.

28: Apparatus according to claim 3, wherein the control unit is adapted to analyze the activity signals by means of a technique selected from the list consisting of: single value decomposition and principal component analysis, and to generate the output signal responsive thereto.

29: Apparatus according to claim 3, wherein the control unit is adapted to analyze the activity signals so as to identify a duration aspect thereof, and to generate the output signal responsive to identifying the duration aspect.

30: Apparatus according to claim 3, wherein the control unit is adapted to analyze the activity signals so as to identify an aspect of morphology of a waveform thereof, and to generate the output signal responsive to identifying the aspect of the morphology.

31: Apparatus according to claim 3, wherein the control unit is adapted to analyze the activity signals so as to identify an aspect of a number of threshold-crossings thereof, and to generate the output signal responsive to identifying the aspect of the number of threshold-crossings.

32: Apparatus according to claim 3, wherein the control unit is adapted to analyze the activity signals using a moving window, and to generate the output signal responsive to the analysis.

33: Apparatus according to claim 3, wherein the control unit is adapted to analyze the activity signals so as to identify a measure of energy thereof, and to generate the output signal responsive to identifying the measure of energy.

34: Apparatus according to claim 3, wherein the control unit is adapted to analyze the activity signals so as to identify a correlation thereof with a stored pattern, and to generate the output signal responsive to identifying the correlation.

35: Apparatus according to claim 3, wherein the control unit is adapted to analyze the activity signals so as to determine an average pattern thereof, and so as to identify a correlation of the activity signals with the average pattern, and wherein the control unit is adapted to generate the output signal responsive to identifying the correlation.

36: Apparatus according to claim 3, wherein the control unit is adapted to analyze the activity signals so as to identify a magnitude aspect thereof and a duration aspect thereof, wherein the control unit is adapted to analyze the aspects in combination, and wherein the control unit is adapted to generate the output signal responsive to analyzing the aspects.

37: Apparatus according to claim 3, wherein the control unit is adapted to analyze the activity signals so as to determine a measure of organization of the activity signals.

38: Apparatus according to claim 3, wherein a first electrode and a second electrode of the set of electrodes are adapted to be coupled to a first site and a second site of the pancreas, respectively, and wherein the control unit is adapted to measure a delay between sensed electrical activity at the first and second sites, and to analyze the activity signals responsive to the measured delay.

39: Apparatus according to claim 3, wherein the control unit is adapted to detect mechanical artifacts by identifying a pattern of the activity signals, the pattern selected from the list consisting of: a spectral pattern and a time pattern.

40: Apparatus according to claim 3, wherein the control unit comprises a memory, and wherein the control unit is adapted to store the activity signals in the memory for subsequent off-line analysis.

41: Apparatus according to claim 3, wherein the control unit is adapted to receive the activity signals from at least one of the electrodes when the at least one of the electrodes is not in physical contact with any islet of the pancreas.

42: Apparatus according to claim 3, wherein the control unit is adapted to receive the activity signals from at least one of the electrodes when the at least one of the electrodes is not in physical contact with the pancreas.

43: Apparatus according to claim 3, wherein the control unit is adapted to generate the output signal so as to facilitate an evaluation of a state of the patient.

44: Apparatus according to claim 3, wherein the set of electrodes comprises at least ten electrodes.

45: Apparatus according to claim 3, wherein the set of electrodes comprises at least 50 electrodes.

46: Apparatus according to claim 3, comprising a clip mount, coupled to at least one of the electrodes, which is adapted for securing the at least one of the electrodes to the pancreas.

47: Apparatus according to claim 3, wherein at least one of the electrodes is adapted to be physically coupled to the pancreas by
peeling back a portion of connective tissue surrounding the pancreas, so as to create a pocket,
inserting the electrode into the pocket, and
suturing the electrode to the connective tissue.

48: Apparatus according to claim 3, wherein the set of one or more electrodes comprises an array of electrodes, the array comprising at least two electrodes adapted to be coupled to the pancreas at respective sites, and adapted to generate an impedance-indicating signal responsive to a level of electrical impedance between the two sites.

49: Apparatus according to claim 3, comprising at least one supplemental sensor, adapted to
be coupled to a site of a body of the patient,
sense a parameter of the patient, and
generate a supplemental signal responsive to the parameter,
and wherein the control unit is adapted to receive the supplemental signal.

50: Apparatus according to claim 49, wherein the parameter is selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate, and wherein the supplemental sensor is adapted to sense the parameter.

51: Apparatus according to claim 50, wherein the metabolic indicator includes a measure of NADH, and wherein the supplemental sensor is adapted to sense the measure of NADH.

52: Apparatus according to claim 49, wherein the supplemental sensor comprises an accelerometer, adapted to detect a motion of an organ of the patient.

53: Apparatus according to claim 49, wherein the control unit is adapted to apply to the activity signals a noise reduction algorithm, an input of which includes the supplemental signal.

54: Apparatus according to claim 3, wherein the control unit is adapted to analyze the activity signals so as to identify a magnitude aspect thereof, and to generate the output signal responsive to identifying the magnitude aspect.

55: Apparatus according to claim 54, wherein the control unit is adapted to analyze the activity signals so as to identify the magnitude aspect thereof at a frequency, and to generate the output signal responsive to identifying the magnitude aspect at the frequency.

56: Apparatus according to claim 3, wherein the control unit is adapted to apply a Fourier transform to the activity signals.

57: Apparatus according to claim 56, wherein the control unit is adapted to analyze the Fourier-transformed activity signals so as to calculate a ratio of (a) a first frequency component at a first frequency of the activity signals to (b) a second frequency component at a second frequency of the activity signals, the first frequency different from the second frequency, and wherein the control unit is adapted to generate the output signal responsive to the analysis.

58: Apparatus according to claim 56, wherein the control unit is adapted to analyze the Fourier-transformed activity signals so as to identify a pattern thereof, and to generate the output signal responsive to identifying the pattern.

59: Apparatus according to claim 3, wherein the control unit is adapted to analyze the activity signals so as to identify an aspect of a frequency of spike generation thereof, and to generate the output signal responsive to identifying the aspect.

60: Apparatus according to claim 59, wherein the control unit is adapted to analyze the activity signals so as to identify the aspect of the frequency of spike generation responsive to an occurrence of spikes within a certain range of durations of spikes, and to generate the output signal responsive to the aspect.

61: Apparatus according to claim 59, wherein the control unit is adapted to analyze the activity signals so as to identify the aspect of the frequency of spike generation responsive to a ratio of spikes with a first amplitude to spikes with a second amplitude, the first amplitude different from the second amplitude, and to generate the output signal responsive to the aspect.

62: Apparatus according to claim 59, wherein the control unit is adapted to analyze the activity signals so as to identify the aspect of the frequency of spike generation responsive to, for each spike, a product of a duration of the spike and an amplitude of the spike, and to generate the output signal responsive to the aspect.

63: Apparatus according to claim 59, wherein the control unit is adapted to analyze the activity signals so as to identify a change in the aspect of the frequency of spike generation, and to generate the output signal responsive to identifying the change in the aspect of the frequency.

64: Apparatus according to claim 3, wherein the control unit is adapted to analyze the activity signals so as to determine a change in a rate of secretion of insulin by the pancreas.

65: Apparatus according to claim 64, wherein the control unit is adapted to determine a change in a rate of spike generation, so as to determine the change in the rate of secretion of insulin by the pancreas.

66: Apparatus according to claim 3, wherein the control unit is adapted to analyze the activity signals with respect to calibration data indicative of aspects of pancreatic electrical activity recorded at respective times, in which respective measurements of a parameter of the patient generated respective values.

67: Apparatus according to claim 66, wherein the parameter includes a blood glucose level of the patient, and wherein the control unit is adapted to analyze the activity signals with respect to the calibration data.

68: Apparatus according to claim 66, wherein the parameter includes a blood insulin level of the patient, and wherein the control unit is adapted to analyze the activity signals with respect to the calibration data.

69: Apparatus according to claim 3, comprising at least one reference electrode, adapted to be coupled to tissue in a vicinity of the pancreas, and to generate reference signals, and wherein the control unit is adapted to receive the reference signals, and to generate the output signal responsive to the reference signals and the activity signals.

70: Apparatus according to claim 69, wherein the reference electrode is adapted to be coupled to an organ of the patient in a vicinity of the pancreas, and to generate reference signals indicative of a motion of the organ.

71: Apparatus according to claim 70, wherein the organ includes a stomach of the patient, and wherein the reference electrode comprises two reference electrodes, adapted to be coupled to the stomach at respective stomach sites, and adapted to generate an impedance-indicating signal responsive to a level of electrical impedance between the two stomach sites.

72: Apparatus according to claim 70, wherein the organ includes a pancreas of the patient, and wherein the reference electrode comprises two reference electrodes, adapted to be coupled to the pancreas at respective pancreas sites, and adapted to generate an impedance-indicating signal responsive to a level of electrical impedance between the two pancreas sites.

73: Apparatus according to claim 70, wherein the organ includes a duodenum of the patient, and wherein the reference electrode comprises two reference electrodes, adapted to be coupled to the duodenum at respective duodenum sites, and adapted to generate an impedance-indicating signal responsive to a level of electrical impedance between the two duodenum sites.

74: Apparatus according to claim 3, wherein the electrodes are adapted to be placed in physical contact with the pancreas.

75: Apparatus according to claim 74, wherein at least one of the electrodes is adapted to be placed in physical contact with the head of the pancreas.

76: Apparatus according to claim 74, wherein at least one of the electrodes is adapted to be placed in physical contact with the body of the pancreas.

77: Apparatus according to claim 74, wherein at least one of the electrodes is adapted to be placed in physical contact with the tail of the pancreas.

78: Apparatus according to claim 74, wherein at least one of the electrodes is adapted to be placed in physical contact with a vein or artery of the pancreas.

79: Apparatus according to claim 3, wherein at least one of the electrodes is adapted to be placed in physical contact with a blood vessel in a vicinity of the pancreas.

80: Apparatus according to claim 3, wherein at least one of the electrodes has a characteristic diameter less than about 3 millimeters.

81: Apparatus according to claim 80, wherein the at least one of the electrodes has a characteristic diameter less than about 300 microns.

82: Apparatus according to claim 81, wherein the at least one of the electrodes has a characteristic diameter less than about 30 microns.

83: Apparatus according to claim 3, wherein the apparatus comprises a treatment unit, adapted to receive the output signal and to apply a treatment to the patient responsive to the output signal.

84: Apparatus according to claim 83, wherein the control unit is adapted to generate the output signal responsive to an aspect of timing of the activity signals, and wherein the treatment unit is adapted to apply the treatment responsive to the timing aspect.

85: Apparatus according to claim 84, wherein the control unit is adapted to generate the output signal responsive to an aspect of the timing of the activity signals indicative of a phase in an oscillation of an insulin level.

86: Apparatus according to claim 83, comprising at least one supplemental sensor, adapted to
be coupled to a site of a body of the patient,
sense a parameter of the patient, and
generate a supplemental signal responsive to the parameter,
and wherein the control unit is adapted to receive the supplemental signal, and to generate the output signal responsive to the supplemental signal and the activity signals, and wherein the treatment unit is adapted to apply the treatment responsive to the output signal.

87: Apparatus according to claim 86, wherein the supplemental sensor comprises an accelerometer, adapted to detect a motion of an organ of the patient.

88: Apparatus according to claim 86, wherein the parameter is selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate, and wherein the supplemental sensor is adapted to sense the parameter.

89: Apparatus according to claim 88, wherein the metabolic indicator includes a measure of NADH, and wherein the supplemental sensor is adapted to sense the measure of NADH.

90: Apparatus according to claim 83, wherein the control unit is adapted to configure the output signal to the treatment unit so as to be capable of modifying an amount of glucose in blood in the patient.

91: Apparatus according to claim 90, wherein the control unit is adapted to configure the output signal to the treatment unit so as to be capable of increasing an amount of glucose in blood in the patient.

92: Apparatus according to claim 90, wherein the control unit is adapted to configure the output signal so as to be capable of decreasing an amount of glucose in blood in the patient.

93: Apparatus according to claim 83, wherein the treatment unit comprises a signal-application electrode, and wherein the control unit is adapted to drive the signal-application electrode to apply current to the pancreas capable of treating a condition of the patient.

94: Apparatus according to claim 93, wherein the signal-application electrode comprises at least one electrode of the set of electrodes.

95: Apparatus according to claim 93, wherein the control unit is adapted to drive the signal-application electrode to apply the current in a waveform selected from the list consisting of: a monophasic square wave pulse, a sinusoid wave, a series of biphasic square waves, and a waveform including an exponentially-varying characteristic.

96: Apparatus according to claim 93, wherein the signal-application electrode comprises a first and a second signal-application electrode, and wherein the control unit is adapted to drive the first and second signal-application electrodes to apply the current in different waveforms.

97: Apparatus according to claim 93, wherein the control unit is adapted to drive the signal-application electrode to apply the current so as to modulate insulin secretion by the pancreas.

98: Apparatus according to claim 97, wherein the control unit is adapted to select a parameter of the current, and to drive the signal-application electrode to apply the current, so as to modulate insulin secretion, the parameter selected from the list consisting of: a magnitude of the current, a duration of the current, and a frequency of the current.

99: Apparatus according to claim 97, wherein the signal-application electrode comprises a first and a second signal-application electrode, and wherein the control unit is adapted to drive the first and the second signal-application electrodes to reverse a polarity of the current applied to the pancreas so as to stimulate the change in insulin secretion.

100: Apparatus according to claim 93, wherein the treatment unit comprises a substance delivery unit, adapted to deliver a therapeutic substance to the patient, and wherein the control unit is adapted to drive the signal-application electrode to apply the current, and, in combination, to drive the substance delivery unit to deliver the therapeutic substance.

101: Apparatus according to claim 83, wherein the treatment unit comprises a patient-alert unit, adapted to generate a patient-alert signal.

102: Apparatus according to claim 83, wherein the treatment unit comprises a substance delivery unit, adapted to deliver a therapeutic substance to the patient.

103: Apparatus according to claim 102, wherein the substance delivery unit comprises a pump.

104: Apparatus according to claim 102, wherein the substance includes insulin, and wherein the substance delivery unit is adapted to deliver the insulin to the patient.

105: Apparatus according to claim 102, wherein the substance includes a drug, and wherein the substance delivery unit is adapted to deliver the drug to the patient.

106: Apparatus according to claim 105, wherein the drug is selected from the list consisting of: glyburide, glipizide, and chlorpropamide.

107: Apparatus for sensing electrical activity of a pancreas of a patient, comprising an electrode assembly, which comprises:
one or more wire electrodes, each wire electrode comprising a curved portion, which curved portion is adapted to be brought in contact with the pancreas, and each wire electrode adapted to generate an activity signal indicative of electrical activity of pancreatic cells which are in a plurality of islets of the pancreas; and
a clip mount, to which the wire electrodes are fixed, which is adapted to secure the wire electrodes to the pancreas.

108: Apparatus for sensing electrical activity of a pancreas of a patient, comprising an electrode assembly, which comprises:
a plurality of wire electrodes, adapted to be brought in contact with and to penetrate a surface of the pancreas, and to generate respective activity signals indicative of electrical activity of pancreatic cells which are in a plurality of islets of the pancreas; and
a mount, to which the wire electrodes are fixed, which is adapted to secure the wire electrodes to the pancreas.

109: Apparatus for sensing electrical activity of a pancreas of a patient, comprising a patch assembly, which comprises:
a patch, adapted to be coupled to tissue of the patient in a vicinity of the pancreas; and
one or more electrode assemblies, adapted to be coupled to the patch such that the electrode assemblies are in electrical contact with the tissue, and adapted to generate respective activity signals indicative of electrical activity of pancreatic cells which are in a plurality of islets of the pancreas.

110: Apparatus according to claim 109, comprising a balloon, coupled to a surface of the patch not in contact with the tissue.

111: Apparatus according to claim 109, comprising a hydrogel, adapted to be applied to a surface of the patch not in contact with the tissue, so as to flexibly harden and maintain coupling of the patch to the tissue.

112: Apparatus according to claim 109, comprising a sheet, coupled to a surface of the patch not in contact with the tissue, so as to protect the patch from motion of organs of the patient.

113: Apparatus according to claim 109, wherein the patch is adapted to have one or more sutures pass therethrough, to couple the patch to the tissue.

114: Apparatus according to claim 109, comprising an adhesive, adapted to couple the patch to the tissue.

115: Apparatus according to claim 109, wherein the electrode assemblies comprise two electrode assemblies, adapted to facilitate a differential measurement of the electrical activity of the pancreas.

116: Apparatus according to claim 109, wherein each of the electrode assemblies comprises:
a wire electrode; and
an insulating ring, surrounding the wire electrode.

117: Apparatus according to claim 109, wherein the patch comprises one or more signal-processing components fixed thereto.

118: Apparatus according to claim 117, wherein at least one of the signal-processing components is selected from the list consisting of: a preamplifier, a filter, an amplifier, an analog-to-digital converter, a preprocessor, and a transmitter.

119: Apparatus according to claim 117, wherein at least one of the signal-processing components is adapted to drive at least one of the electrode assemblies to apply a signal to a portion of the tissue, the signal configured so as to treat a condition of the patient.

120: Apparatus according to claim 109, wherein each of the electrode assemblies comprises:
an inner wire electrode, adapted to function as a first pole of the electrode assembly;
an inner insulating ring, adapted to surround the inner wire electrode;
an outer ring electrode, adapted to surround the inner insulating ring, and to function as a second pole of the electrode assembly; and
an outer insulating ring, adapted to surround the outer ring electrode.

121: Apparatus according to claim 120, wherein the inner wire electrode is adapted to have a tissue-contact surface area approximately equal to a tissue-contact surface area of the outer ring electrode.

122: Apparatus, comprising a patch, adapted to be implanted in contact with tissue of a patient, the tissue in a vicinity of a pancreas of the patient, the patch comprising one or more signal-processing components fixed thereto, which are adapted to process pancreatic electrical signals.

123: Apparatus according to claim 122, wherein at least one of the signal-processing components is selected from the list consisting of: a preamplifier, a filter, an amplifier, an analog-to-digital converter, a preprocessor, and a transmitter.

124: Apparatus according to claim 122, wherein the tissue includes tissue of the pancreas of the patient, and wherein the patch is adapted to be coupled to the tissue of the pancreas.

125: Apparatus according to claim 122, wherein the tissue includes tissue of a duodenum of the patient, and wherein the patch is adapted to be coupled to the tissue of the duodenum.

126: Apparatus according to claim 122, comprising an electrode, adapted
to be coupled to tissue of the patient in a vicinity of the pancreas,
to generate an activity signal indicative of electrical activity of pancreatic cells which are in a plurality of islets of the pancreas, and
to be electrically coupled to at least one of the signal-processing components.

127: Apparatus according to claim 126, wherein at least one of the signal-processing components is adapted to drive the electrode to apply a signal to the pancreas, the signal configured so as to treat a condition of the patient.

128: Apparatus for sensing electrical activity of a pancreas of a patient, comprising:
a patch, adapted to be coupled to first tissue of the patient in a vicinity of the pancreas, the patch comprising a signal-processing component;
at least one electrode assembly, comprising:
an electrode, adapted to be coupled to second tissue of the patient in a vicinity of the pancreas and in a vicinity of the patch, and to generate an activity signal indicative of electrical activity of pancreatic cells which are in a plurality of islets of the pancreas; and
a wire having a first end and a second end, the first end physically and electrically coupled to the electrode, the second end comprising a surgical needle, adapted to be electrically coupled to the second end, the wire adapted to function as a suture for use with the needle, and the second end adapted to be physically and electrically coupled to the preamplifier.

129: Apparatus according to claim 128, wherein the signal-processing component comprises a preamplifier.

130: Apparatus according to claim 129, wherein the second end is adapted to be physically and electrically coupled to the preamplifier by inserting the needle into the preamplifier.

131: Apparatus according to claim 129, wherein the needle is adapted to be broken after the wire is sutured to the second tissue, thereby leaving a broken portion of the needle fixed to the second end of the wire, and wherein the second end of the wire is adapted to be physically and electrically coupled to the preamplifier by inserting the broken portion of the needle into the preamplifier.

132: Apparatus for sensing electrical activity of a pancreas of a patient, comprising an electrode, adapted to be coupled to tissue of the patient in a vicinity of the pancreas, and adapted to generate an activity signal indicative of electrical activity of pancreatic cells which are in a plurality of islets of the pancreas, the electrode comprising a hooking element, which comprises a plurality of prongs, the prongs adapted to be collapsible while being inserted into the tissue, and to expand after insertion, thereby generally securing the electrode in the tissue.

133: Apparatus for sensing electrical activity of a pancreas of a patient, comprising an electrode, adapted to be coupled to tissue of the patient in a vicinity of the pancreas, and adapted to generate an activity signal indicative of electrical activity of pancreatic cells which are in a plurality of islets of the pancreas, the electrode comprising a spiral stopper element, adapted to secure the electrode in the tissue.

134: Apparatus for sensing electrical activity of a pancreas of a patient, comprising an electrode, adapted to be coupled to tissue of the patient in a vicinity of the pancreas, and adapted to generate an activity signal indicative of electrical activity of pancreatic cells which are in a plurality of islets of the pancreas, the electrode comprising a corkscrew element, adapted to secure the electrode in the tissue.

135: Apparatus for sensing electrical activity of a pancreas of a patient, comprising an electrode assembly, comprising:
a connecting element;
an amplifier;
at least two wires, each wire having a proximal end and a distal end, the distal end of each wire adapted to be attached to the connecting element, and the proximal end of each wire adapted to be attached to the amplifier, each wire comprising an electrically-insulating coating attached thereto, adapted to cover a portion of the wire and to not cover at least one exposed site on the wire, so as to provide electrical contact between the exposed site and tissue of the pancreas; and
a suture, having a proximal end and a distal end, the proximal end adapted to be attached to the amplifier, and the distal end adapted to be connected to the connecting element.

136: Apparatus according to claim 135, wherein one of the exposed sites on a first one of the wires and one of the exposed sites on a second one of the wires are adapted to facilitate a differential measurement of the electrical activity of the pancreas.

137: Apparatus according to claim 135, comprising a needle, attached to the distal end of the suture.

138: Apparatus for analyzing electrical activity of a pancreas of a patient, comprising:
a set of one or more electrodes, adapted to be coupled to the pancreas and to generate respective activity signals indicative of electrical activity of pancreatic cells; and
a control unit, adapted to:
receive the activity signals from the one or more electrodes,
analyze a frequency component of the received activity signals, and
generate an output signal responsive to the analysis.

139: Apparatus for analyzing activity of a pancreas of a patient, comprising:
a set of one or more calcium electrodes, each of the calcium electrodes adapted to be coupled to the pancreas and to generate a signal indicative of a calcium level; and
a control unit, adapted to:
receive the signals from the one or more calcium electrodes,
analyze the received activity signals, and
generate an output signal responsive to the analysis.

140: Apparatus according to claim 139, wherein each of the electrodes is adapted to generate the signal indicative of an intracellular calcium level.

141: Apparatus according to claim 139, wherein each of the electrodes is adapted to generate the signal indicative of an interstitial calcium level.

142: A method for sensing electrical activity of a pancreas of a patient, comprising:
sensing electrical activity of pancreatic cells which are in a plurality of islets of the pancreas;
generating activity signals responsive thereto;
receiving the activity signals;
analyzing the activity signals; and
generating an output signal responsive to the analysis.

143: A method according to claim 142, wherein sensing the electrical activity comprises sensing, at a single site of the pancreas, electrical activity of pancreatic cells which are in two or more of the islets.

144: A method for sensing electrical activity of a pancreas of a patient, comprising:
sensing, at each of one or more sites of the pancreas, electrical activity of pancreatic cells in a respective plurality of islets;
generating activity signals responsive thereto;
receiving the activity signals;
analyzing the activity signals; and
generating an output signal responsive to the analysis.

145: A method according to claim 144, wherein receiving the activity signals comprises receiving signals indicative of electrical activity of pancreatic cells which are in five or more of the islets.

146: A method according to claim 144, wherein receiving the activity signals comprises receiving signals indicative of electrical activity of pancreatic cells which are in ten or more of the islets.

147: A method according to claim 144, wherein receiving the activity signals comprises:
receiving a first activity signal recorded at a first site, indicative of electrical activity of pancreatic cells which are in a first one of the islets; and
receiving a second activity signal recorded at a second site, indicative of electrical activity of pancreatic cells which are in a second one of the islets, which is different from the first one of the islets.

148: A method according to claim 144, wherein analyzing the activity signals comprises analyzing the activity signals so as to identify an aspect thereof indicative of activity of a type of cell selected from the list consisting of: pancreatic alpha cells, pancreatic beta cells, pancreatic delta cells, and polypeptide cells, and wherein generating the output signal comprises generating the output signal responsive to identifying the aspect.

149: A method for monitoring a blood glucose level of a patient, comprising:
sensing spontaneous electrical activity of pancreatic cells;
generating activity signals responsive thereto;
receiving the activity signals;
analyzing the activity signals so as to determine a change in the glucose level; and
generating an output signal responsive to determining the change.

150: A method for monitoring a blood insulin level of a patient, comprising:
sensing spontaneous electrical activity of pancreatic cells;
generating activity signals responsive thereto;
receiving the activity signals;
analyzing the activity signals so as to determine a change in the insulin level; and
generating an output signal responsive to determining the change.

151: A method according to claim 149, wherein analyzing the activity signals comprises identifying an aspect thereof indicative of activity of a type of cell selected from the list consisting of: pancreatic alpha cells, pancreatic beta cells, pancreatic delta cells, and polypeptide cells, and wherein generating the output signal comprises generating the output signal responsive to identifying the aspect.

152: A method according to claim 144, wherein analyzing the activity signals comprises analyzing the activity signals so as to identify a frequency aspect thereof, and wherein generating the output signal comprises generating the output signal responsive to identifying the frequency aspect.

153: A method for analyzing electrical activity of a pancreas of a patient, comprising:
sensing electrical activity at one or more pancreatic sites;
generating activity signals responsive thereto;
receiving the activity signals;
analyzing the activity signals so as to identify an aspect thereof which is indicative of activity of pancreatic alpha cells; and
generating an output signal responsive to identifying the aspect.

154: A method for analyzing electrical activity of a pancreas of a patient, comprising:
sensing electrical activity at one or more pancreatic sites;
generating activity signals responsive thereto;
receiving the activity signals;
analyzing the activity signals so as to identify an aspect thereof which is indicative of activity of pancreatic beta cells; and
generating an output signal responsive to identifying the aspect.

155: A method according to claim 154, wherein analyzing the activity signals comprises distinguishing between the aspect thereof which is indicative of the activity of the beta cells and an aspect thereof which is indicative of activity of pancreatic alpha cells, and wherein generating the output signal comprises generating the output signal responsive to distinguishing between the aspects.

156: A method for analyzing electrical activity of a pancreas of a patient, comprising:
sensing electrical activity at one or more pancreatic sites;
generating activity signals responsive thereto;
receiving the activity signals;
analyzing the activity signals so as to identify an aspect thereof which is indicative of activity of pancreatic delta cells; and
generating an output signal responsive to identifying the aspect.

157: A method for analyzing electrical activity of a pancreas of a patient, comprising:
sensing electrical activity at one or more pancreatic sites;
generating activity signals responsive thereto;
receiving the activity signals;
analyzing the activity signals so as to identify an aspect thereof which is indicative of activity of polypeptide cells; and
generating an output signal responsive to identifying the aspect.

158: A method according to claim 154, wherein analyzing the activity signals comprises comparing the aspect of the activity signals with a stored pattern that is indicative of activity of the cells, and wherein generating the output signal comprises generating the output signal responsive thereto.

159: A method according to claim 154, wherein analyzing the activity signals comprises analyzing the activity signals under an assumption that the activity of the cells is dependent on electrical activity of another type of pancreatic cell, and wherein generating the output signal comprises generating the output signal responsive thereto.

160: A method according to claim 154, wherein analyzing the activity signals comprises analyzing the activity signals under an assumption that the activity of the cells is substantially independent of electrical activity of another type of pancreatic cell, and wherein generating the output signal comprises generating the output signal responsive thereto.

161: A method according to claim 154, wherein analyzing the activity signals comprises analyzing the activity signals so as to identify a frequency aspect thereof, and wherein generating the output signal comprises generating the output signal responsive to identifying the frequency aspect.

162: A method according to claim 161, wherein analyzing the activity signals comprises analyzing the activity signals so as to differentiate between a first frequency aspect of the activity signals which is indicative of the activity of the cells, and a second frequency aspect of the activity signals, different from the first frequency aspect, which is indicative of activity of another type of pancreatic cell.

163: A method according to claim 161, wherein analyzing the activity signals comprises analyzing the activity signals so as to identify over time a change in the frequency aspect that is characteristic of the cells.

164: A method according to claim 161, wherein analyzing the activity signals comprises:
analyzing the activity signals so as to identify a magnitude aspect thereof; and
analyzing the frequency aspect and the magnitude aspect in combination,
wherein generating the output signal comprises generating the output signal responsive to analyzing the aspects.

165: A method according to claim 161, wherein analyzing the activity signals comprises:
analyzing the activity signals so as to identify a duration aspect thereof; and
analyzing the frequency aspect and the duration aspect in combination,
wherein generating the output signal comprises generating the output signal responsive to analyzing the aspects.

166: A method according to claim 144, wherein receiving the activity signals comprises receiving electrical signals responsive to spontaneous electrical activity of the pancreatic cells.

167: A method according to claim 144, wherein receiving the activity signals comprises receiving activity signals recorded at at least ten pancreatic sites.

168: A method according to claim 144, wherein analyzing the activity signals comprises analyzing the activity signals by means of a technique selected from the list consisting of: single value decomposition and principal component analysis, and wherein generating the output signal comprises generating the output signal responsive thereto.

169: A method according to claim 144, wherein analyzing the activity signals comprises analyzing the activity signals so as to identify an aspect of morphology of a waveform thereof, and wherein generating the output signal comprises generating the output signal responsive to identifying the aspect of the morphology.

170: A method according to claim 144, wherein analyzing the activity signals comprises analyzing the activity signals so as to identify an aspect of a number of threshold-crossings thereof, and wherein generating the output signal comprises generating the output signal responsive to identifying the aspect of the number of threshold-crossings.

171: A method according to claim 144, wherein analyzing the activity signals comprises analyzing the activity signals using a moving window, and wherein generating the output signal comprises generating the output signal responsive to the analysis.

172: A method according to claim 144, wherein analyzing the activity signals comprises analyzing the activity signals so as to identify a measure of energy thereof, and wherein generating the output signal comprises generating the output signal responsive to identifying the measure of energy.

173: A method according to claim 144, wherein analyzing the activity signals comprises analyzing the activity signals so as to identify a correlation thereof with a stored pattern, and wherein generating the output signal comprises generating the output signal responsive to identifying the correlation.

174: A method according to claim 144, wherein analyzing the activity signals comprises analyzing the activity signals so as to determine an average pattern thereof, and so as to identify a correlation of the activity signals with the average pattern, and wherein generating the output signal comprises generating the output signal responsive to identifying the correlation.

175: A method according to claim 144, comprising applying a synchronizing signal to the pancreas, so as to synchronize pancreatic beta cell depolarization.

176: A method according to claim 144, wherein analyzing the activity signals comprises analyzing the activity signals so as to identify a magnitude of a fluctuation of the activity signals, and wherein generating the output signal comprises generating the output signal responsive to the analysis.

177: A method according to claim 144, wherein analyzing the activity signals comprises analyzing the activity signals so as to identify a duration aspect thereof, and wherein generating the output signal comprises generating the output signal responsive to identifying the duration aspect.

178: A method according to claim 144, wherein analyzing the activity signals comprises:
analyzing the activity signals so as to identify a magnitude aspect thereof and a duration aspect thereof; and
analyzing the aspects in combination,
wherein generating the output signal comprises generating the output signal responsive to analyzing the aspects.

179: A method according to claim 144, wherein analyzing the activity signals comprises analyzing the activity signals so as to determine a measure of organization of the activity signals.

180: A method according to claim 144, wherein receiving the activity signals comprises receiving activity signals generated at a first site and at a second site of the pancreas, and wherein analyzing the activity signals comprises measuring a delay between sensed electrical activity at the first and second sites, and analyzing the activity signals responsive to the measured delay.

181: A method according to claim 144, wherein analyzing the activity signals comprises detecting mechanical artifacts.

182: A method according to claim 181, wherein detecting the mechanical artifacts comprises identifying a pattern of the activity signals, the pattern selected from the list consisting of: a spectral pattern and a time pattern.

183: A method according to claim 144, comprising storing the activity signals for subsequent off-line analysis.

184: A method according to claim 144, wherein generating the output signal comprises facilitating an evaluation of a state of the patient.

185: A method according to claim 144, wherein receiving the activity signals comprises receiving the activity signals from at least one electrode not in physical contact with the pancreas.

186: A method according to claim 144, wherein receiving the activity signals comprises receiving the activity signals from at least one electrode which is not in physical contact with any islet of the pancreas.

187: A method according to claim 144, wherein receiving the activity signals comprises receiving the activity signals from at least one electrode which is in physical contact with a blood vessel in a vicinity of the pancreas.

188: A method according to claim 144, wherein analyzing the activity signals comprises analyzing the activity signals so as to identify a magnitude aspect thereof, and wherein generating the output signal comprises generating the output signal responsive to identifying the magnitude aspect.

189: A method according to claim 188, wherein the magnitude aspect includes a magnitude of a frequency component of the activity signals, and wherein generating the output signal comprises generating the output signal responsive to the magnitude of the frequency component.

190: A method according to claim 144, wherein analyzing the activity signals comprises applying a Fourier transform to the activity signals.

191: A method according to claim 190, wherein analyzing the activity signals comprises analyzing the Fourier-transformed activity signals so as to calculate a ratio of (a) a first frequency component at a first frequency of the activity signals to (b) a second frequency component at a second frequency of the activity signals, the first frequency different from the second frequency, and wherein generating the output signal comprises generating the output signal responsive to the analysis.

192: A method according to claim 190, wherein analyzing the activity signals comprises analyzing the Fourier-transformed activity signals so as to identify a pattern thereof, and wherein generating the output signal comprises generating the output signal responsive to identifying the pattern.

193: A method according to claim 144, wherein analyzing the activity signals comprises analyzing the activity signals so as to identify an aspect of a frequency of spike generation thereof, and wherein generating the output signal comprises generating the output signal responsive to identifying the aspect.

194: A method according to claim 193, wherein analyzing the activity signals comprises analyzing the activity signals so as to identify the aspect of the frequency of spike generation responsive to an occurrence of spikes within a determined range of durations of spikes.

195: A method according to claim 193, wherein analyzing the activity signals comprises analyzing the activity signals so as to identify the aspect of the frequency of spike generation responsive to a ratio of spikes with a first amplitude to spikes with a second amplitude, the first amplitude different from the second amplitude.

196: A method according to claim 193, wherein analyzing the activity signals comprises analyzing the activity signals so as to identify the aspect of the frequency of spike generation responsive to, for each spike, a product of a duration of the spike and an amplitude of the spike.

197: A method according to claim 193, wherein analyzing the activity signals comprises analyzing the activity signals so as to identify a change in the aspect of the frequency of spike generation, and wherein generating the output signal comprises generating the output signal responsive to identifying the change in the aspect of the frequency.

198: A method according to claim 144, wherein analyzing the activity signals comprises determining a change in a rate of secretion of insulin by the pancreas.

199: A method according to claim 198, wherein analyzing the activity signals comprises determining a change in a rate of spike generation, so as to determine the change in the rate of secretion of insulin by the pancreas.

200: A method according to claim 144, comprising:
sensing a parameter of the patient at a site in a body of the patient;
generating a supplemental signal responsive to the parameter; and
receiving the supplemental signal.

201: A method according to claim 200, wherein sensing the parameter comprises sensing a parameter selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate.

202: A method according to claim 201, wherein sensing the parameter comprises sensing a measure of NADH.

203: A method according to claim 200, wherein analyzing the activity signals comprises applying to the activity signals a noise reduction algorithm, an input of which includes the supplemental signal.

204: A method according to claim 144, wherein analyzing the activity signals comprises analyzing the activity signals with respect to calibration data indicative of aspects of pancreatic electrical activity recorded at respective times, in which respective measurements of a parameter of the patient generated respective values.

205: A method according to claim 204, wherein the parameter includes a blood glucose level of the patient, and wherein analyzing the activity signals comprises analyzing the activity signals with respect to the calibration data.

206: A method according to claim 204, wherein the parameter includes a blood insulin level of the patient, and wherein analyzing the activity signals comprises analyzing the activity signals with respect to the calibration data.

207: A method according to claim 144, comprising:
sensing an electrical parameter of tissue in a vicinity of the pancreas;
generating reference signals responsive thereto; and
receiving the reference signals,
wherein generating the output signal comprises generating the output signal responsive to the reference signals and the activity signals.

208: A method according to claim 207, wherein sensing the electrical parameter of the tissue comprises driving a current between two sites of an organ including the tissue,
wherein sensing the electrical parameter comprises sensing the electrical parameter responsive to driving the current and responsive to an electrical impedance between the two sites, and
wherein generating the reference signals comprises generating reference signals indicative of a motion of the organ, responsive to the electrical parameter.

209: A method according to claim 208, wherein the organ includes a stomach of the patient, and wherein sensing the electrical parameter comprises driving the current between two sites of the stomach.

210: A method according to claim 208, wherein the organ includes a pancreas of the patient, and wherein sensing the electrical parameter comprises driving the current between two sites of the pancreas.

211: A method according to claim 208, wherein the organ includes a duodenum of the patient, and wherein sensing the electrical parameter comprises driving the current between two sites of the duodenum.

212: A method according to claim 144, wherein receiving the activity signals comprises receiving the activity signals from at least one electrode placed in physical contact with the pancreas.

213: A method according to claim 212, wherein receiving the activity signals comprises receiving the activity signals from at least one electrode placed in physical contact with the head of the pancreas.

214: A method according to claim 212, wherein receiving the activity signals comprises receiving the activity signals from at least one electrode placed in physical contact with the body of the pancreas.

215: A method according to claim 212, wherein receiving the activity signals comprises receiving the activity signals from at least one electrode placed in physical contact with the tail of the pancreas.

216: A method according to claim 212, wherein receiving the activity signals comprises receiving the activity signals from at least one electrode placed in physical contact with a vein or artery of the pancreas.

217: A method according to claim 144, comprising applying a treatment to the patient responsive to the output signal.

218: A method according to claim 217, wherein applying the treatment comprises applying the treatment responsive to an aspect of the timing of the activity signals.

219: A method according to claim 217, wherein applying the treatment comprises generating a patient-alert signal.

220: A method according to claim 217, comprising:
sensing a parameter of the patient at a site in a body of the patient;
generating a supplemental signal responsive to the parameter; and
receiving the supplemental signal,
wherein generating the output signal comprises generating the output signal responsive to the supplemental signal and the activity signals.

221: A method according to claim 220, wherein sensing the parameter comprises sensing a parameter selected from the list consisting of: blood sugar, SvO2, pH, pCO2, pO2, blood insulin levels, blood ketone levels, ketone levels in expired air, blood pressure, respiration rate, respiration depth, an electrocardiogram measurement, a metabolic indicator, and heart rate.

222: A method according to claim 221, wherein sensing the parameter comprises sensing a measure of NADH.

223: A method according to claim 217, wherein applying the treatment comprises configuring the treatment so as to be capable of modifying an amount of glucose in blood in the patient.

224: A method according to claim 223, wherein configuring the treatment comprises configuring the treatment so as to be capable of increasing an amount of glucose in blood in the patient.

225: A method according to claim 223, wherein configuring the treatment comprises configuring the treatment so as to be capable of decreasing an amount of glucose in blood in the patient.

226: A method according to claim 217, wherein applying the treatment comprises applying electric current to the pancreas capable of treating a condition of the patient.

227: A method according to claim 226, wherein applying the electric current comprises applying the electric current in a waveform selected from the list consisting of: a monophasic square wave pulse, a sinusoid wave, a series of biphasic square waves, and a waveform including an exponentially-varying characteristic.

228: A method according to claim 226, wherein applying the electric current comprises applying the electric current in different waveforms at a first and a second site of the pancreas.

229: A method according to claim 226, wherein applying the electric current comprises applying the electric current so as to modulate insulin secretion by the pancreas.

230: A method according to claim 229, wherein applying the electric current comprises reversing a polarity of the electric current so as to modulate insulin secretion.

231: A method according to claim 217, wherein applying the treatment comprises delivering a therapeutic substance to the patient.

232: A method according to claim 231, wherein the substance includes insulin, and wherein delivering the substance comprises delivering the insulin to the patient.

233: A method according to claim 231, wherein the substance includes a drug, and wherein delivering the substance comprises delivering the drug to the patient.

234: A method according to claim 233, wherein the drug is selected from the list consisting of: glyburide, glipizide, and chlorpropamide.

235: A method for coupling an electrode to a pancreas of a patient, comprising:
peeling back a portion of connective tissue surrounding the pancreas, so as to create a pocket;
inserting the electrode into the pocket; and
suturing the electrode to the connective tissue.

236: A method for sensing electrical activity of a pancreas of a patient, comprising:
sensing, at each of one or more sites of the pancreas, electrical activity of pancreatic cells;
generating activity signals responsive thereto;
receiving the activity signals;
analyzing a frequency component of the activity signals; and
generating an output signal responsive to the analysis.

237: A method for sensing activity of a pancreas of a patient, comprising:
sensing, at each of one or more sites of the pancreas, a calcium level;
generating activity signals responsive thereto;
receiving the activity signals;
analyzing the activity signals; and
generating an output signal responsive to the analysis.

238: A method according to claim 237, wherein sensing the calcium level comprises sensing an intracellular calcium level.

239: A method according to claim 237, wherein sensing the calcium level comprises sensing an interstitial calcium level.
